(19) United States
(12) Patent Application Publication
(10) Pub. No.: US 2024/0009220 A1
(43) Pub. Date: Jan. 11, 2024

(54) METHODS FOR TREATMENT OF VIRAL INFECTIONS

(71) Applicant: Gilead Sciences, Inc., Foster City, CA (US)

(72) Inventors: Roy Maxim Bannister, Morgan Hill, CA (US); John P. Bilello, Redwood City, CA (US); Jared D. Pitts, Foster City, CA (US)

(21) Appl. No.: 18/205,745

(22) Filed: Jun. 5, 2023

Related U.S. Application Data
(60) Provisional application No. 63/349,531, filed on Jun. 6, 2022.
(60) Provisional application No. 63/461,218, filed on Apr. 21, 2023.

(51) Int. Cl. A61K 31/706; A61K 31/427; A61K 31/4025; A61P 31/14

(52) U.S. Cl. CPC A61K 31/706; A61K 31/427; A61K 31/4025; A61P 31/14

(57) ABSTRACT

The present disclosure relates to methods for treating viral infections in a patient that is not pregnant. Also provided are combination treatments for viral infections in a human in need thereof.

### CROSS-REFERENCES TO RELATED APPLICATIONS

This application claims the benefit of U.S. Provisional Application No. 63/349,531, filed on Jun. 6, 2022 and U.S. Provisional Application No. 63/461,218, filed on Apr. 21, 2023. The entire contents of these applications are incorporated herein by reference in their entirety.

### FIELD OF THE INVENTION

The present disclosure relates to methods for treating viral infections.

### BACKGROUND

There is a need for compounds and methods for treating viral infections, for example paramyxoviridae, pneumoviridae, picornaviridae, flaviviridae, filoviridae, arenaviridae, orthomyxovirus, and coronaviridae infections.

### SUMMARY

Provided herein is a method of treating a viral infection in a patient in need thereof, wherein the method comprises administering to the patient a compound of Formula A:

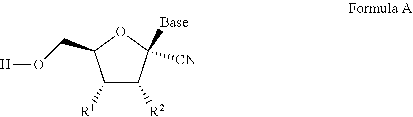

a deuterated compound of Formula A, a prodrug of the compound of Formula A, a prodrug of the deuterated compound of Formula A, or a pharmaceutically acceptable salt thereof, wherein R¹, R², and Base are defined herein, wherein when the prodrug of the compound of Formula A, the prodrug of the deuterated compound of Formula A, or the pharmaceutically acceptable salt thereof is administered, the prodrug of the compound of Formula A, the prodrug of the deuterated compound of Formula A, or the pharmaceutically acceptable salt thereof is converted substantially to the compound of Formula A or the deuterated compound of Formula A, and wherein the patient is not a pregnant individual.

Also provided herein is a method of treating a viral infection in a human in need thereof, the method comprising administering to the human
(i) Compound 1:

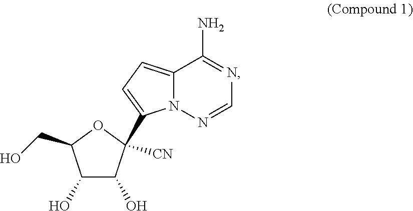 [SMILES: N#C[C@@]1(c2ccc3c(N)ncnn23)O[C@H](CO)[C@@H](O)[C@H]1O]

a deuterated Compound 1, a prodrug of Compound 1, a prodrug of deuterated Compound 1, or a pharmaceutically acceptable salt thereof, and
(ii) nirmatrelvir, ritonavir, or a combination of nirmatrelvir and ritonavir;
   wherein when the prodrug of Compound 1, the prodrug of deuterated Compound 1, or the pharmaceutically acceptable salt thereof is administered to the human, the prodrug of Compound 1, the prodrug of deuterated Compound 1, or the pharmaceutically acceptable salt thereof is converted substantially to Compound 1 or deuterated Compound 1.

### BRIEF DESCRIPTION OF THE FIGURES

FIG. 1 shows a representative two-drug combination bliss independence consensus plots for a A549-hACE-2 SARS-CoV-2 fluc antiviral analysis for Compound 1 and Nirmatrelvir.

FIG. 2 shows a representative three-drug combination bliss independence consensus plots for a A549-hACE-2 SARS-CoV-2 fluc antiviral analysis for Compound 1, Nirmatrelvir, and Ritonavir.

### DETAILED DESCRIPTION

Unless stated otherwise, the following terms and phrases as used herein are intended to have the following meanings:

“Alkyl” refers to an unbranched or branched saturated hydrocarbon chain. For example, an alkyl group can have 1 to 20 carbon atoms (i.e., C₁-C₂₀ alkyl), 1 to 8 carbon atoms (i.e., C₁-C₈ alkyl), 1 to 6 carbon atoms (i.e., C₁-C₆ alkyl), or 1 to 3 carbon atoms (i.e., C₁-C₃ alkyl). Examples of suitable alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), and 3,3-dimethyl-2-butyl (—CH(CH₃)C(CH₃)₃.

“Alkenyl” refers to an aliphatic group containing at least one carbon-carbon double bond and having from 2 to 20 carbon atoms (i.e., C₂₋₂₀ alkenyl), 2 to 8 carbon atoms (i.e., C₂₋₈ alkenyl), 2 to 6 carbon atoms (i.e., C₂₋₆ alkenyl), or 2 to 4 carbon atoms (i.e., C₂₋₄ alkenyl). Examples of alkenyl groups include ethenyl, propenyl, butadienyl (including 1,2-butadienyl and 1,3-butadienyl).

“Alkynyl” refers to an aliphatic group containing at least one carbon-carbon triple bond and having from 2 to 20 carbon atoms (i.e., C₂₋₂₀ alkynyl), 2 to 8 carbon atoms (i.e., C₂₋₈ alkynyl), 2 to 6 carbon atoms (i.e., C₂₋₆ alkynyl), or 2 to 4 carbon atoms (i.e., C₂₋₄ alkynyl). The term “alkynyl” also includes those groups having one triple bond and one double bond.

“Haloalkyl” is an alkyl group, as defined above, in which one or more hydrogen atoms of the alkyl group is replaced with a halogen atom. The alkyl portion of a haloalkyl group can have 1 to 20 carbon atoms (i.e., C₁-C₂₀ haloalkyl), 1 to 12 carbon atoms (i.e., C₁-C₁₂ haloalkyl), 1 to 8 carbon atoms (i.e., C₁-C₈ haloalkyl), 1 to 6 carbon atoms (i.e., C₁-C₆ alkyl) or 1 to 3 carbon atoms (i.e., C₁-C₃ alkyl). Examples of suitable haloalkyl groups include, but are not limited to, —CF₃, —CHF₂, —CFH₂, —CH₂CF₃, and the like.

“Aryl” means an aromatic hydrocarbon radical derived by the removal of one hydrogen atom from a single carbon atom of a parent aromatic ring system. For example, an aryl group can have 6 to 20 carbon atoms, 6 to 14 carbon atoms, or 6 to 10 carbon atoms. Typical aryl groups include, but are not limited to, radicals derived from benzene (e.g., phenyl), substituted benzene, naphthalene, anthracene, biphenyl, and the like.

“Heteroaryl” refers to an aromatic group having a single ring, multiple rings, or multiple fused rings, with one or more ring heteroatoms independently selected from nitrogen, oxygen, and sulfur. As used herein, heteroaryl includes 1 to 20 ring atoms (i.e., 1 to 20 membered heteroaryl), 3 to 12 ring atoms (i.e., 3 to 12 membered heteroaryl) or 3 to 8 carbon ring atoms (3 to 8 membered heteroaryl) or 5 to 6 ring atoms (5 to 6 membered heteroaryl). Examples of heteroaryl groups include pyrimidinyl, purinyl, pyridyl, pyridazinyl, benzothiazolyl, and pyrazolyl. Heteroaryl does not encompass or overlap with aryl as defined above.

“Carbocyclyl” or “carbocyclic ring” refers to a non-aromatic hydrocarbon ring consisting of carbon and hydrogen atoms, having from three to twenty carbon atoms, in certain embodiments having from three to fifteen carbon atoms, in certain embodiments having from three to ten carbon atoms, from three to eight carbon atoms, from three to seven carbon atoms, or from 3 to 6 carbon atoms and which is saturated or partially unsaturated and attached to the rest of the molecule by a single bond. Carbocyclic rings include, for example, cyclopropane, cyclobutane, cyclopentane, cyclopentene, cyclohexane, cyclohexene, 1,3-cyclohexadiene, 1,4-cyclohexadiene, cycloheptane, cycloheptene, and cyclooctane. Carbocyclic rings include cycloalkyl groups.

“Cycloalkyl” refers to a saturated cyclic alkyl group having a single ring or multiple rings including fused, bridged, and spiro ring systems. As used herein, cycloalkyl has from 3 to 20 ring carbon atoms (i.e., C₃₋₂₀ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Examples of cycloalkyl groups include cyclopropyl, cyclobutyl, cyclopentyl, cyclohexyl, cycloheptyl, and cyclooctyl.

“Heterocycle” or “heterocyclyl” as used herein includes by way of example and not limitation those heterocycles described in Paquette, Leo A.; Principles of Modern Heterocyclic Chemistry (W. A. Benjamin, New York, 1968), particularly Chapters 1, 3, 4, 6, 7, and 9; “The Chemistry of Heterocyclic Compounds, A Series of Monographs” (John Wiley & Sons, New York, 1950 to present), in particular Volumes 13, 14, 16, 19, and 28; and J. Am. Chem. Soc. (1960) 82:5566. For example, “heterocycle” includes a “carbocycle” as defined herein, wherein one or more (e.g. 1, 2, 3, or 4) carbon atoms have been replaced with a heteroatom (e.g. O, N, or S). As used herein, heterocycle or heterocyclyl has from 3 to 20 ring atoms, 3 to 12 ring atoms, 3 to 10 ring atoms, 3 to 8 ring atoms, or 3 to 6 ring atoms. The terms “heterocycle” or “heterocyclyl” includes saturated rings and partially unsaturated rings. Substituted heterocyclyls include, for example, heterocyclic rings substituted with any of the substituents described herein including carbonyl groups. A non-limiting example of a carbonyl substituted heterocyclyl is:

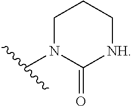 [SMILES: *N1CCCNC1=O]

Example heterocycles include, but are not limited to, tetrahydrofuranyl azetidinyl, and 2-oxo-1,3-dioxol-4-yl.

The term “optionally substituted” in reference to a particular moiety of the compound described herein such as the compound of Formula A or Formula I (e.g., an optionally substituted aryl group) refers to a moiety wherein all substituents are hydrogen or wherein one or more of the hydrogens of the moiety may be replaced by the listed substituents.

Unless otherwise specified, the carbon atoms of the compounds of Formula and Formula I are intended to have a valence of four. If in some chemical structure representations, carbon atoms do not have a sufficient number of variables attached to produce a valence of four, the remaining carbon substituents needed to provide a valence of four should be assumed to be hydrogen.

Any reference to the compounds described herein also includes a reference to a pharmaceutically acceptable salt thereof. Examples of pharmaceutically acceptable salts of the compounds described herein include salts derived from an appropriate base, such as an alkali metal or an alkaline earth (for example, Na⁺, Li⁺, K⁺, Ca⁺² and Mg⁺²), ammonium and NR₄⁺ (wherein R is defined herein). Pharmaceutically acceptable salts of a nitrogen atom or an amino group include (a) acid addition salts formed with inorganic acids, for example, hydrochloric acid, hydrobromic acid, sulfuric acid, sulfamic acids, phosphoric acid, nitric acid and the like; (b) salts formed with organic acids such as, for example, acetic acid, oxalic acid, tartaric acid, succinic acid, maleic acid, fumaric acid, gluconic acid, citric acid, malic acid, ascorbic acid, benzoic acid, isethionic acid, lactobionic acid, tannic acid, palmitic acid, alginic acid, polyglutamic acid, naphthalenesulfonic acid, methanesulfonic acid, p-toluenesulfonic acid, benzenesulfonic acid, naphthalenedisulfonic acid, polygalacturonic acid, malonic acid, sulfosalicylic acid, glycolic acid, 2-hydroxy-3-naphthoate, pamoate, salicylic acid, stearic acid, phthalic acid, mandelic acid, lactic acid, ethanesulfonic acid, lysine, arginine, glutamic acid, glycine, serine, threonine, alanine, isoleucine, leucine and the like; and (c) salts formed from elemental anions for example, chlorine, bromine, and iodine. Pharmaceutically acceptable salts of a compound of a hydroxy group include the anion of said compound in combination with a suitable cation such as Na⁺ and NR₄⁺. In some embodiments, R₄ is H, (C₁-C₈) alkyl, (C₂-C₈)alkenyl, (C₂-C₈) alkynyl, C₆-C₂₀ aryl, or C₂-C₂₀ heterocyclyl.

For therapeutic use, salts of active ingredients of the compounds described herein will be pharmaceutically acceptable, i.e., they will be salts derived from a pharmaceutically acceptable acid or base. It is also to be understood that the compositions herein comprise compounds described herein in their un-ionized, as well as zwitterionic form, and combinations with stoichiometric amounts of water as in hydrates. It is to be noted that all enantiomers, diastereomers, racemic mixtures, tautomers, polymorphs, and pseudopolymorphs of compounds described herein (e.g., compounds within the scope of Formula A or Formula I) and pharmaceutically acceptable salts thereof are embraced by the present disclosure. All mixtures of such enantiomers and diastereomers are within the scope of the present disclosure.

The compounds described herein may have chiral centers, e.g., chiral carbon or phosphorus atoms. The compounds described herein thus include racemic mixtures of all stereoisomers, including enantiomers, diastereomers, and atropisomers. In addition, the compounds described herein include enriched or resolved optical isomers at any or all asymmetric, chiral atoms. In other words, the chiral centers apparent from the depictions are provided as the chiral isomers or racemic mixtures. Both racemic and diastereomeric mixtures, as well as the individual optical isomers isolated or synthesized, substantially free of their enantiomeric or diastereomeric partners, are all within the scope of the disclosure. The racemic mixtures are separated into their individual, substantially optically pure isomers through appropriate techniques such as, for example, the separation of diastereomeric salts formed with optically active adjuncts, e.g., acids or bases followed by conversion back to the optically active substances. In most instances, the desired optical isomer is synthesized by means of stereospecific reactions, beginning with the appropriate stereoisomer of the desired starting material.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes d and l, D and L, or (+) and (−) are employed to designate the sign of rotation of plane-polarized light by the compound, with S, (−), or 1 meaning that the compound is levorotatory while a compound prefixed with R, (+), or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

The compounds described herein may also exist as tautomeric isomers in certain cases. Although only one delocalized resonance structure may be depicted, all such forms are contemplated within the scope of the invention. For example, ene-amine tautomers can exist for purine, pyrimidine, imidazole, guanidine, amidine, and tetrazole systems and all their possible tautomeric forms are within the scope of the invention.

Any formula or structure described herein, including Formula A and Formula I compounds, is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl and ¹²⁵I. Various isotopically labeled compounds of the present disclosure, for example those into which radioactive isotopes such as ³H, ¹³C and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The disclosure also includes compounds (e.g., compounds of Formula A or Formula I) in which from 1 to x hydrogens attached to a carbon atom is/are replaced by deuterium, in which x is the number of hydrogens in the molecule. Such compounds exhibit increased resistance to metabolism and are thus useful for increasing the half-life of any compound described herein (e.g., compounds of Formula A or Formula I) when administered to a mammal, particularly a human. See, for example, Foster, “Deuterium Isotope Effects in Studies of Drug Metabolism”, Trends Pharmacol. Sci. 5(12):524-527 (1984). In view of the present disclosure, such compounds are synthesized by means known in the art, for example by employing starting materials in which one or more hydrogens have been replaced by deuterium.

Deuterium labeled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F labeled compound may be useful for PET or SPECT studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in the compounds described herein.

For example, in the deuterated compound of Formula A, one or more hydrogen atoms attached to one or more carbon atoms of Formula A are replaced by deuterium. In some embodiments of the deuterated compound of Formula A, one hydrogen atom attached to one carbon atom of Formula A is replaced by deuterium. In some embodiments of the deuterated compound of Formula A, two hydrogen atoms attached to one carbon atom of Formula A are replaced by deuterium. In some embodiments of the deuterated compound of Formula A, two or more hydrogen atoms attached to two carbon atoms of Formula A are replaced by deuterium.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

Whenever a compound described herein is substituted with more than one of the same designated group, e.g., “R” or “R”, then it will be understood that the groups may be the same or different, i.e., each group is independently selected.

Wavy lines, [symbol] , indicate the site of covalent bond attachments to the adjoining substructures, groups, moieties, or atoms.

The term “treating”, as used herein, unless otherwise indicated, means reversing, alleviating, or inhibiting the progress of the disorder or condition to which such term applies, or one or more symptoms of such disorder or condition. The term “treatment”, as used herein, refers to the act of treating, as “treating” is defined immediately above.

“Prevention” or “preventing” means any treatment of a disease or condition that causes the clinical symptoms of the disease or condition not to develop. The compounds and compositions described herein may, in some embodiments, be administered to a subject (including a human) who is at risk of having the disease or condition. As used herein, the terms “preventing” and “prevention” encompass the administration of a compound, composition, or pharmaceutically acceptable salt according to the embodiments described herein pre- or post-exposure of the individual to a virus, but before the appearance of symptoms of the viral infection, and/or prior to the detection of the virus in the blood. The terms also refer to prevention of the appearance of symptoms of the disease and/or to prevent the virus from reaching detectible levels in the blood. The terms include both pre-exposure prophylaxis (PrEP), as well as post-exposure prophylaxis (PEP) and event driven or “on demand” prophylaxis. The terms also refer to prevention of perinatal transmission of a virus from mother to baby, by administration to the mother before giving birth and to the child within the first days of life. The terms also refer to prevention of transmission of a virus through blood transfusion.

The term “therapeutically effective amount”, as used herein, is the amount of a compound described herein (e.g., a compound of Formula A or Formula I) present in a composition described herein that is needed to provide a desired level of drug in the secretions and tissues of the airways and lungs, or alternatively, in the bloodstream of a subject to be treated to give an anticipated physiological response or desired biological effect when such a composition is administered by the chosen route of administration. The precise amount will depend upon numerous factors, for example the particular compound described herein (e.g., the compound of Formula A or Formula I), the specific activity of the composition, the delivery device employed, the physical characteristics of the composition, its intended use, as well as patient considerations such as severity of the disease state, patient cooperation, etc., and can readily be determined by one skilled in the art based upon the information provided herein.

The term “prodrug,” as used herein, refers to a biologically inactive derivative of a drug that, upon administration to the patient, can be converted to a parent drug according to some chemical or enzymatic pathway. For example, when a prodrug of the compound of Formula A, or a pharmaceutically acceptable salt thereof, is administered, the prodrug, or the pharmaceutically acceptable salt thereof, can be converted to the compound of Formula A. In another example, when a prodrug of the deuterated compound of Formula A, or a pharmaceutically acceptable salt thereof, is administered, the prodrug, or the pharmaceutically acceptable salt thereof, can be converted to the deuterated compound of Formula A.

The term “converted substantially,” as used herein in reference to a prodrug, refers to conversion of greater than 50% of a prodrug (e.g., a prodrug of the compound of Formula A, or a prodrug of the deuterated compound of Formula A) to a parent compound (e.g., the compound of Formula A, or the deuterated compound of Formula A). For example, the term “converted substantially” can refer to conversion of greater than 50%, greater than 60%, greater than 70%, greater than 80%, greater than 90%, greater than 95%, or greater than 99% of the prodrug of the compound of Formula A, the prodrug of the deuterated compound of Formula A, or the pharmaceutically acceptable salt thereof, to the compound of Formula A or the deuterated compound of Formula A.

The term “symptom” as used herein, can include symptoms of viral infections including, for example, fever, chills, cough, shortness of breath, fatigue, myalgias, headache, diarrhea, loss of taste, loss of smell, sore throat, congestion, nausea, and vomiting.

Reference will now be made in detail to certain embodiments of the invention, examples of which are illustrated in the accompanying description. While the invention will be described in conjunction with the enumerated embodiments, it will be understood that they are not intended to limit the invention to those embodiments. On the contrary, the invention is intended to cover all alternatives, modifications, and equivalents, which may be included within the scope of the present invention.

Provided is a method of treating a viral infection in a patient in need thereof, wherein the method comprises administering to the patient a compound of Formula A:

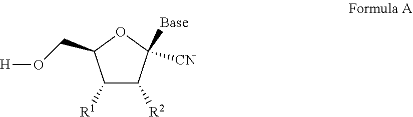

a deuterated compound of Formula A, a prodrug of the compound of Formula A, a prodrug of the deuterated compound of Formula A, or a pharmaceutically acceptable salt thereof, wherein:
R¹ is —OH, —OC(═O)R⁴, or —OC(═O)OR⁴;
R² is —OH, —OC(═O)R⁵, or —OC(═O)OR⁵; or
R¹ and R² are taken together to form —OC(═O)O—, —OP(═O)(OH)O—, or —OCHR⁶O—;
R⁴ and R⁵ are each independently H, C₁-C₈ alkyl, C₂-C₈ alkenyl, C₂-C₈ alkynyl, C₃-C₈ carbocyclyl, C₆-C₁₀ aryl, 4 to 6 membered heterocyclyl containing 1, 2, or 3 heteroatoms selected from N, O, and S, or 5 to 6 membered heteroaryl containing 1, 2, or 3 heteroatoms selected from N, O, and S; wherein each C₁-C₈ alkyl, C₂-C₈ alkenyl, C₂-C₈ alkynyl, C₃-C₈ carbocyclyl, C₆-C₁₀ aryl, 4 to 6 membered heterocyclyl, and 5 to 6 membered heteroaryl of R⁴ and R⁵ is independently, optionally substituted with one, two, or three R^{a} substituents;
R⁶ is H, C₁-C₆ alkyl, C₁-C₆ alkoxy, 5 to 6 membered heteroaryl containing 1, 2, or 3 heteroatoms selected from N, O, and S, or C₆-C₁₀ aryl; wherein each 5 to 6 membered heteroaryl and C₆-C₁₀ aryl of R⁶ is independently, optionally substituted with one, two, or three R^{b} substituents;
Base is

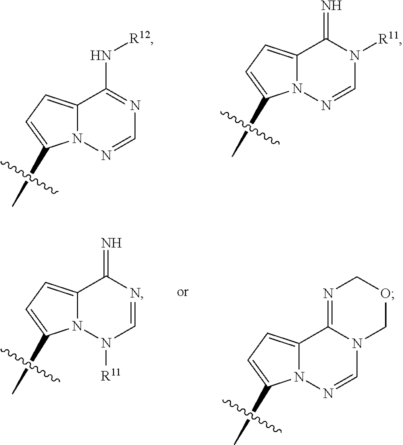

R¹¹ is C₁-C₆ alkyl optionally substituted with —OP(═O)(OH)(OR¹⁴);
R¹² is H, C₁-C₆ alkyl, —C(═O)R¹³ or —C(═O)OR¹³;
each R¹³ is independently H, C₁-C₁₀ alkyl, C₆-C₁₀ aryl, —O—C₆-C₁₀ aryl, or —O—C₁-C₁₀ alkyl; wherein each C₁-C₁₀ alkyl, C₆-C₁₀ aryl, —O—C₆-C₁₀ aryl, and —O—C₁-C₁₀ alkyl of R¹³ is independently, optionally substituted with one, two, or three R^{c} substituents;
each R^{a} is independently halo, cyano, C₁-C₆ alkyl, carbonyl, —N₃, —OR⁸, —NR⁹R¹⁰, —OP(═O)(OH)₂, C₃-C₈ carbocyclyl, 4 to 6 membered heterocyclyl containing 1, 2, or 3 heteroatoms selected from N, O, and S, 5 to 6 membered heteroaryl containing 1, 2, or 3 heteroatoms selected from N, O, and S, or phenyl; wherein each 4 to 6 membered heterocyclyl of R^{a} is independently, optionally substituted with one, two, or three R^{d} substituents; wherein each phenyl of R^{a} is independently, optionally substituted with one, two or three R^{e} substituents;
R⁸, R⁹, and R¹⁰ are each independently H, C₁-C₆ alkyl, C₁-C₆ haloalkyl, or C₃-C₆ cycloalkyl
each R^{b} is independently halo, cyano, C₁-C₆ alkoxy, or C₁-C₆ alkyl;
each R^{c} is independently halo, cyano, —OP(═O)(OH)(OR¹⁴) or phenyl; wherein each phenyl of R^{c} is optionally substituted with —OP(═O)(OH)(OR¹⁴);
each R¹⁴ is independently H, C₁-C₈ alkyl, C₃-C₈ carbocyclyl, C₆-C₁₀ aryl, or 5 to 6 membered heteroaryl containing 1, 2, or 3 heteroatoms selected from N, O, and S; wherein each C₁-C₈ alkyl of R¹⁴ is independently, optionally substituted with one, two or three R^{f} substituents;
each R^{d} is independently carbonyl or C₁-C₆ alkyl;
each R^{e} is independently halo, cyano, or C₁-C₆ alkyl; and
each R^{f} is independently halo, cyano, or phenyl;
wherein when the prodrug of the compound of Formula A, the prodrug of the deuterated compound of Formula A, or the pharmaceutically acceptable salt thereof is administered, the prodrug of the compound of Formula A, the prodrug of the deuterated compound of Formula A, or the pharmaceutically acceptable salt thereof is converted substantially to the compound of Formula A or the deuterated compound of Formula A; and
wherein the patient is not a pregnant individual.

In some embodiments, upon administration to the patient, greater than 60% of the prodrug of the compound of Formula A, the prodrug of the deuterated compound of Formula A, or the pharmaceutically acceptable salt thereof is converted to the compound of Formula A or the deuterated compound of Formula A. In some embodiments, upon administration to the patient, greater than 70% of the prodrug of the compound of Formula A, the prodrug of the deuterated compound of Formula A, or the pharmaceutically acceptable salt thereof is converted to the compound of Formula A or the deuterated compound of Formula A. In some embodiments, upon administration to the patient, greater than 80% of the prodrug of the compound of Formula A, the prodrug of the deuterated compound of Formula A, or the pharmaceutically acceptable salt thereof is converted to the compound of Formula A or the deuterated compound of Formula A. In some embodiments, upon administration to the patient, greater than 90% of the prodrug of the compound of Formula A, the prodrug of the deuterated compound of Formula A, or the pharmaceutically acceptable salt thereof is converted to the compound of Formula A or the deuterated compound of Formula A. In some embodiments, upon administration to the patient, greater than 95% of the prodrug of the compound of Formula A, the prodrug of the deuterated compound of Formula A, or the pharmaceutically acceptable salt thereof is converted to the compound of Formula A or the deuterated compound of Formula A. In some embodiments, upon administration to the patient, greater than 95% of the prodrug of the compound of Formula A, the prodrug of the deuterated compound of Formula A, or the pharmaceutically acceptable salt thereof is converted to the compound of Formula A or the deuterated compound of Formula A.

In some embodiments, the carbon bonded to the 5 position on the tetrahydrofuranyl ring of the deuterated compound of Formula A is substituted with one or two deuterium atoms. In some embodiments, the deuterated compound of Formula A is

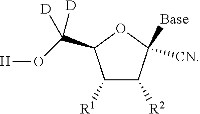

In some embodiments, the deuterated compound of Formula A is

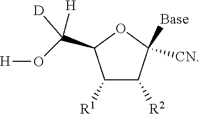

In some embodiments, a carbon of the Base of the deuterated compound of Formula A is substituted with one or more deuterium atoms. In some embodiments, Base is

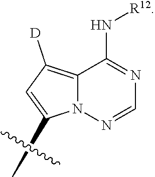

In some embodiments, Base is

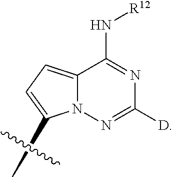

In some embodiments, Base is

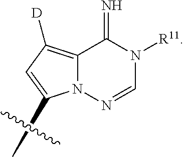

In some embodiments, Base is

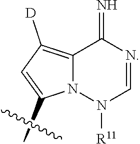

In some embodiments, a carbon of R¹² of the Base of the deuterated compound of Formula A is substituted with one or more deuterium atoms, e.g., one or two deuterium atoms. In some embodiments, a carbon on R¹¹ of the Base of the deuterated compound of Formula A is substituted with one or more deuterium atoms, e.g., one or two deuterium atoms.

In some embodiments, a carbon of R¹ of the deuterated compound of Formula A is substituted with one or more deuterium atoms, e.g., one or two deuterium atoms. In some embodiments, a carbon of R² of the deuterated compound of Formula A is substituted with one or more deuterium atoms, e.g., one or two deuterium atoms.

In some embodiments, the compound of Formula A is

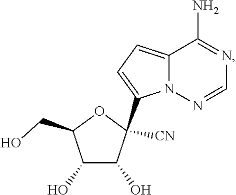 [SMILES: N#C[C@@]1(c2ccc3c(N)ncnn23)O[C@H](CO)[C@@H](O)[C@H]1O]

or a pharmaceutically acceptable salt thereof.

In some embodiments, the deuterated compound of Formula A is

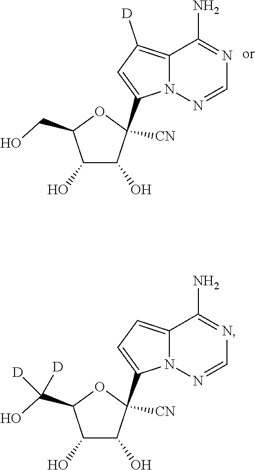 [SMILES: [2H]c1cc([C@]2(C#N)O[C@H](CO)[C@@H](O)[C@H]2O)n2ncnc(N)c12 ; [2H]C([2H])(O)[C@H]1O[C@@](C#N)(c2ccc3c(N)ncnn23)[C@H](O)[C@@H]1O]

or a pharmaceutically acceptable salt thereof.

In some embodiments, the method comprises administering to the patient a prodrug of Formula A having Formula I:

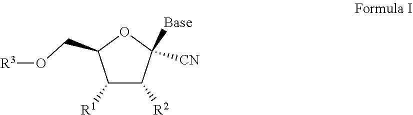

or a deuterated compound of Formula I, or a pharmaceutically acceptable salt thereof, wherein:
R³ is —C(═O)OR⁷, or —C(═O)R⁷; and
R⁷ is H, C₁-C₈ alkyl, C₂-C₈ alkenyl, C₂-C₈ alkynyl, C₃-C₈ carbocyclyl, C₆-C₁₀ aryl, 4 to 6 membered heterocyclyl containing 1, 2, or 3 heteroatoms selected from N, O, and S, or 5 to 6 membered heteroaryl containing 1, 2, or 3 heteroatoms selected from N, O, and S; wherein C₁-C₈ alkyl, C₂-C₈ alkenyl, C₂-C₈ alkynyl, C₃-C₈ carbocyclyl, C₆-C₁₀ aryl, 4 to 6 membered heterocyclyl, and 5 to 6 membered heteroaryl of R⁷ are optionally substituted with one, two, or three R^{a} substituents.

In some embodiments, a carbon of R³ of the deuterated compound of Formula I is substituted with one or more deuterium atoms, e.g., one or two deuterium atoms. In some embodiments, a carbon of R⁷ of the deuterated compound of Formula I is substituted with one or more deuterium atoms, e.g., one or two deuterium atoms.

In some embodiments, R¹ is —OH. In some embodiments, R¹ is —OC(═O)R⁴. In some embodiments, R¹ is —OC(═O)OR⁴.

In some embodiments, R² is —OH. In some embodiments, R² is —OC(═O)R⁵. In some embodiments, R² is —OC(═O)OR⁵.

In some embodiments, R¹ and R² are both —OH. In some embodiments, R¹ is —OC(═O)R⁴ and R² is —OC(═O)R⁵. In some embodiments, R¹ is OH, and R² is —OC(═O)R⁵ or —OC(═O)OR⁵. In some embodiments, R¹ is —OC(═O)R⁴ or —OC(═O)OR⁴, and R² is OH. In some embodiments, R¹ and R² are taken together to form —OC(═O)O— In some embodiments, R¹ and R² are taken together to form —OP(═O)(OH)O—. In some embodiments, R¹ and R² are taken together to form —OCHR⁶O—.

In some embodiments,
R¹ is OH, OC(O)CH(CH₃)₂, or OC(O)CH₃,
R² is OH, OC(O)CH(CH₃)₂, or OC(O)CH₃, or
R¹ and R² are taken together to form —OC(═O)O—.

In some embodiments, R⁴ is C₁-C₈ alkyl, C₂-C₈ alkenyl, or C₂-C₈ alkynyl. In some embodiments, R⁴ is C₁-C₈ alkyl, C₂-C₈ alkenyl, or C₂-C₈ alkynyl, each optionally substituted with one, two, or three R^{a} substituents independently selected from halo, cyano, —N₃, —OR⁸, —NR⁹R¹⁰, —OP(═O)(OH)₂, C₃-C₈ carbocyclyl and phenyl.

In some embodiments, R⁴ is C₁-C₈ alkyl. In some embodiments, R⁴ is C₁-C₈ alkyl optionally substituted with one, two, or three R^{a} substituents independently selected from halo, cyano, —N₃, —OR⁸, —NR⁹R¹⁰, —OP(═O)(OH)₂, C₃-C₈ carbocyclyl and phenyl. In some embodiments, R⁴ is unsubstituted C₁-C₈ alkyl. In some embodiments, R⁴ is unsubstituted C₁-C₆ alkyl. In some embodiments, R⁴ is unsubstituted C₁-C₃ alkyl. In some embodiments, R⁴ is —CH₃, —CH₂CH₃, —(CH₂)₂CH₃, —CH(CH₃)₂, —(CH₂)₃CH₃, or —C(CH₃)₃. In some embodiments, R⁴ is —CH₃ or —CH(CH₃)₂.

In some embodiments, R⁵ is C₁-C₈ alkyl, C₂-C₈ alkenyl, or C₂-C₈ alkynyl. In some embodiments, R⁵ is C₁-C₈ alkyl, C₂-C₈ alkenyl, or C₂-C₈ alkynyl, each optionally substituted with one, two, or three R^{a} substituents independently selected from halo, cyano, —N₃, —OR⁸, —NR⁹R¹⁰, —OP(═O)(OH)₂, C₃-C₈ carbocyclyl and phenyl.

In some embodiments, R⁵ is C₁-C₈ alkyl. In some embodiments, R⁵ is C₁-C₈ alkyl optionally substituted with one, two, or three R^{a} substituents independently selected from halo, cyano, —N₃, —OR⁸, —NR⁹R¹⁰, —OP(═O)(OH)₂, C₃-C₈ carbocyclyl and phenyl. In some embodiments, R⁵ is unsubstituted C₁-C₈ alkyl. In some embodiments, R⁵ is unsubstituted C₁-C₆ alkyl. In some embodiments, R⁵ is unsubstituted C₁-C₃ alkyl. In some embodiments, R⁵ is —CH₃, —CH₂CH₃, —(CH₂)₂CH₃, —CH(CH₃)₂, —(CH₂)₃CH₃, or —C(CH₃)₃. In some embodiments, R⁵ is —CH₃ or —CH(CH₃)₂.

In some embodiments, R⁴ and R⁵ are the same. In some embodiments, R⁴ and R⁵ are different. In some embodiments, R⁴ is C₁-C₈ alkyl and R⁵ is C₁-C₈ alkyl. In some embodiments, R⁴ is unsubstituted C₁-C₈ alkyl and R⁵ is unsubstituted C₁-C₈ alkyl. In some embodiments, R⁴ is —CH₃ or —CH(CH₃)₂ and R⁵ is —CH₃ or —CH(CH₃)₂. In some embodiments, R⁴ is —CH₃ and R⁵ is —CH₃. In some embodiments, R⁴ is —CH(CH₃)₂ and R⁵ is —CH(CH₃)₂.

In some embodiments, R⁶ is H. In some embodiments, R⁶ is C₁-C₆ alkyl. In some embodiments, R⁶ is —CH₃, —CH₂CH₃, —(CH₂)₂CH₃, —CH(CH₃)₂, —(CH₂)₃CH₃, or —C(CH₃)₃. In some embodiments, R⁶ is C₁-C₆ alkoxy.

In some embodiments, R⁶ is 5 to 6 membered heteroaryl containing 1, 2, or 3 heteroatoms selected from N, O, and S. In some embodiments, R⁶ is 5 to 6 membered heteroaryl containing 1, 2, or 3 heteroatoms selected from N, O, and S substituted with one, two, or three R^{b} substitutents. In some embodiments, R⁶ is 5 to 6 membered heteroaryl containing 1, 2, or 3 heteroatoms selected from N, O, and S. In some embodiments, R⁶ is unsubstituted 5 to 6 membered heteroaryl containing 1, 2, or 3 heteroatoms selected from N, O, and S.

In some embodiments, R⁶ is C₆-C₁₀ aryl. In some embodiments, R⁶ is C₆-C₁₀ aryl substituted with one, two, or three R^{b} substituents. In some embodiments, R⁶ is unsubstituted C₆-C₁₀ aryl. In some embodiments, R⁶ is phenyl. In some embodiments, R⁶ is unsubstituted phenyl.

In some embodiments, R³ is —C(═O)OR⁷. In some embodiments, R³ is —C(═O)R⁷.

In some embodiments, R⁷ is C₁-C₈ alkyl, C₃-C₈ carbocyclyl, C₆-C₁₀ aryl, 4 to 6 membered heterocyclyl containing 1, 2, or 3 heteroatoms selected from N, O, and S, or 5 to 6 membered heteroaryl containing 1, 2, or 3 heteroatoms selected from N, O, and S. In some embodiments, R⁷ is C₁-C₈ alkyl, C₃-C₈ carbocyclyl, C₆-C₁₀ aryl, 4 to 6 membered heterocyclyl containing 1, 2, or 3 heteroatoms selected from N, O, and S, or 5 to 6 membered heteroaryl containing 1, 2, or 3 heteroatoms selected from N, O, and S, each optionally substituted with one, two, or three R^{a} substituents independently selected from —OR⁸, —OP(═O)(OH)₂, C₃-C₈ carbocyclyl and phenyl.

In some embodiments, R⁷ is C₁-C₈ alkyl, C₂-C₈ alkenyl, or C₂-C₈ alkynyl. In some embodiments, R⁷ is C₁-C₈ alkyl, C₂-C₈ alkenyl, or C₂-C₈ alkynyl, each optionally substituted with one, two, or three R^{a} substituents independently selected from halo, cyano, —N₃, —OR⁸, —NR⁹R¹⁰, —OP(═O)(OH)₂, C₃-C₈ carbocyclyl and phenyl. In some embodiments, R⁷ is C₁-C₈ alkyl, C₂-C₈ alkenyl, or C₂-C₈ alkynyl, each optionally substituted with one, two, or three R^{a} substituents independently selected from halo, cyano, —N₃, —OR⁸, —NR⁹R¹⁰, and phenyl.

In some embodiments, R⁷ is C₁-C₈ alkyl. In some embodiments, R⁷ is C₁-C₈ alkyl optionally substituted with one, two, or three R^{a} substituents independently selected from halo, cyano, —N₃, —OR⁸, —NR⁹R¹⁰, —OP(═O)(OH)₂, C₃-C₈ carbocyclyl and phenyl. In some embodiments, R⁷ is C₁-C₈ alkyl optionally substituted with one R^{a} substituent selected from —OR⁸, —OP(═O)(OH)₂, C₃-C₈ carbocyclyl and unsubstituted phenyl. In some embodiments, R⁷ is C₁-C₈ alkyl optionally substituted with one, two, or three R^{a} substituents independently selected from halo, cyano, —N₃, —OR⁸, —NR⁹R¹⁰, and phenyl. In some embodiments, R⁷ is C₁-C₈ alkyl optionally substituted with one, two, or three R^{a} substituents independently selected from halo, cyano, —N₃, —OR⁸, —NR⁹R¹⁰, and unsubstituted phenyl. In some embodiments, R⁷ is C₁-C₈ alkyl substituted with —OR⁸. In some embodiments, R⁷ is C₁-C₈ alkyl substituted with phenyl. In some embodiments, R⁷ is C₁-C₈ alkyl substituted with C₃-C₈ carbocyclyl. In some embodiments, R⁷ is C₁-C₈ alkyl substituted with —OP(═O)(OH)₂.

In some embodiments, R⁷ is C₁-C₆ alkyl. In some embodiments, R⁷ is C₁-C₆ alkyl optionally substituted with one, two, or three R^{a} substituents independently selected from halo, cyano, —N₃, —OR⁸, —NR⁹R¹⁰, —OP(═O)(OH)₂, C₃-C₈ carbocyclyl and phenyl. In some embodiments, R⁷ is C₁-C₆ alkyl optionally substituted with one R^{a} substituent selected from —OR⁸, —OP(═O)(OH)₂, C₃-C₈ carbocyclyl and unsubstituted phenyl.

In some embodiments, R⁷ is C₁-C₄ alkyl substituted with phenyl. In some embodiments, R⁷ is C₁-C₄ alkyl substituted with phenyl. R⁷ is C₁-C₄ alkyl substituted with —OP(═O)(OH)₂.

In some embodiments, R⁷ is C₂-C₄ alkyl. In some embodiments, R⁷ is C₂-C₄ alkyl substituted with —OR⁸. In some embodiments, R⁷ is C₂-C₄ alkyl substituted with —OCH₃. In some embodiments, R⁷ is C₂-C₄ alkyl substituted with C₄-C₇ carbocyclyl. In some embodiments, R⁷ is C₂-C₄ alkyl substituted with

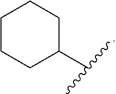 [SMILES: *C1CCCCC1]

In some embodiments, R⁷ is —CH₃, —CH₂CH₃,

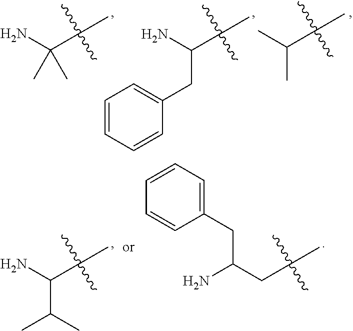

In some embodiments, R⁷—CH₃, —CH₂CH₃,

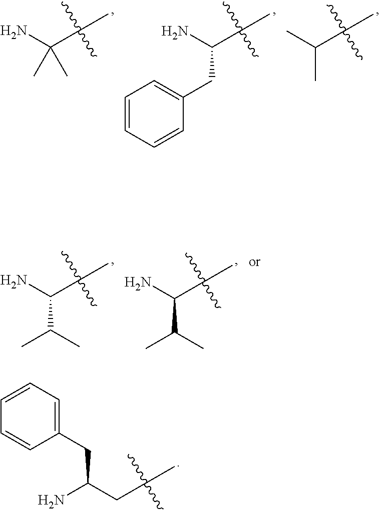

In some embodiments, R⁷ is —CH₃, —CH₂CH₃,

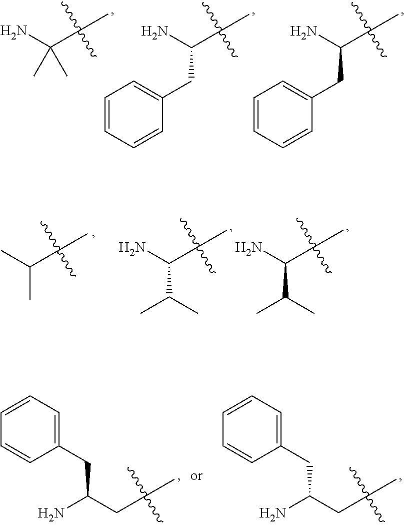

In some embodiments, R⁷ is

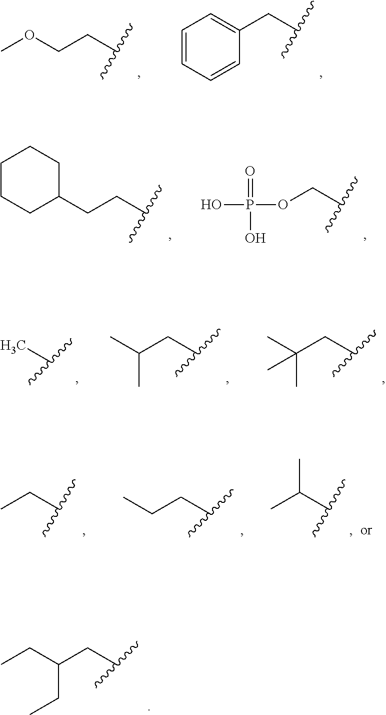

In some embodiments, R⁷ is

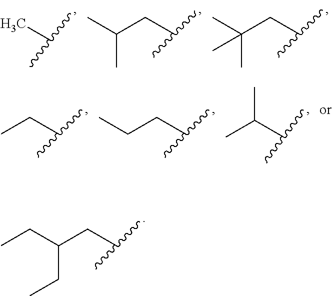

In some embodiments, R⁷ is

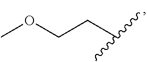 [SMILES: *CCOC]

In some embodiments, R⁷ is

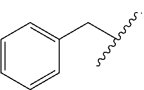 [SMILES: *Cc1ccccc1]

In some embodiments, R⁷ is

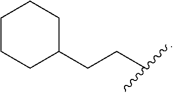 [SMILES: *CCC1CCCCC1]

In some embodiments, R⁷ is

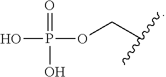 [SMILES: *COP(=O)(O)O]

In some embodiments, R⁷ is C₃-C₈ carbocyclyl. In some embodiments, R⁷ is C₃-C₈ carbocyclyl optionally substituted with one, two, or three R^{a} substituents independently selected from halogen, cyano, —N₃, —OR⁸, —NR⁹R¹⁰, —OP(═O)(OH)₂, C₃-C₈ carbocyclyl and phenyl. In some embodiments, R⁷ is C₃-C₈ carbocyclyl optionally substituted with one, two, or three R^{a} substituents independently selected from halogen, cyano, —N₃, —OR⁸, —NR⁹R¹⁰, —OP(═O)(OH)₂, C₃-C₈ carbocyclyl and unsubstituted phenyl. In some embodiments, R⁷ is C₃-C₈ carbocyclyl optionally substituted with one, two, or three substituents independently selected from —OR⁸, —NR⁹R¹⁰, C₃-C₈ carbocyclyl and unsubstituted phenyl.

In some embodiments, R⁷ is

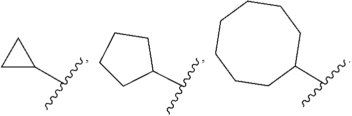

In some embodiments, R⁷ is C₆-C₁₀ aryl. In some embodiments, R⁷ is C₆-C₁₀ aryl optionally substituted with one, two, or three R^{a} substituents independently selected from halogen, cyano, —N₃, —OR⁸, —NR⁹R¹⁰, —OP(═O)(OH)₂, C₃-C₈ carbocyclyl and phenyl. In some embodiments, R⁷ is C₆-C₁₀ aryl optionally substituted with one, two, or three R^{a} substituents independently selected from halogen, cyano, —N₃, —OR⁸, —NR⁹R¹⁰, —OP(═O)(OH)₂, C₃-C₈ carbocyclyl and unsubstituted phenyl.

In some embodiments, R⁷ is phenyl or naphthyl. In some embodiments, R⁷ is phenyl or naphthyl, each optionally substituted with one, two, or three R^{a} substituents independently selected from halogen, cyano, —N₃, —OR⁸, —NR⁹R¹⁰, —OP(═O)(OH)₂, C₃-C₈ carbocyclyl and unsubstituted phenyl. In some embodiments, R⁷ is phenyl. In some embodiments, R⁷ is phenyl optionally substituted with one, two, or three R^{a} substituents independently selected from halogen, cyano, —N₃, —OR⁸, —NR⁹R¹⁰, —OP(═O)(OH)₂, C₃-C₈ carbocyclyl and unsubstituted phenyl.

In some embodiments, R⁷ is

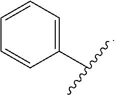 [SMILES: *c1ccccc1]

In some embodiments, R⁷ is 4 to 6 membered heterocyclyl containing 1, 2, or 3 heteroatoms selected from N, O, and S. In some embodiments, R⁷ is 4 to 6 membered heterocyclyl containing 1, 2, or 3 heteroatoms selected from N, O, and S optionally substituted with one, two, or three R^{a} substituents independently selected from halogen, cyano, —N₃, —OR⁸, —NR⁹R¹⁰, —OP(═O)(OH)₂, C₃-C₈ carbocyclyl and phenyl. In some embodiments, R⁷ is 4 to 6 membered heterocyclyl containing 1, 2, or 3 heteroatoms selected from N, O, and S optionally substituted with one, two, or three R^{a} substituents independently selected from halogen, cyano, —N₃, —OR⁸, —NR⁹R¹⁰, —OP(═O)(OH)₂, C₃-C₈ carbocyclyl and unsubstituted phenyl.

In some embodiments, R⁷ is 6 membered heterocyclyl containing 1, 2, or 3 heteroatoms selected from N, O, and S. In some embodiments, R⁷ is 6 membered heterocyclyl containing 1, 2, or 3 heteroatoms selected from N, O, and S optionally substituted with one, two, or three R^{a} substituents independently selected from halogen, cyano, —N₃, —OR⁸, —NR⁹R¹⁰, —OP(═O)(OH)₂, C₃-C₈ carbocyclyl and unsubstituted phenyl.

In some embodiments, R⁷ is 5 membered heterocyclyl containing 1, 2, or 3 heteroatoms selected from N, O, and S. In some embodiments, R⁷ is 5 membered heterocyclyl containing 1, 2, or 3 heteroatoms selected from N, O, and S optionally substituted with one, two, or three R^{a} substituents independently selected from halogen, cyano, —N₃, —OR⁸, —NR⁹R¹⁰, —OP(═O)(OH)₂, C₃-C₈ carbocyclyl and unsubstituted phenyl. In some embodiments, R⁷ is unsubstituted 5 membered heterocyclyl containing 1, 2, or 3 heteroatoms selected from N, O, and S.

In some embodiments, R⁷ is

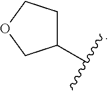 [SMILES: *C1CCOC1]

In some embodiments, R⁷ is 4 membered heterocyclyl containing 1, 2, or 3 heteroatoms selected from N, O, and S. In some embodiments, R⁷ is unsubstituted 4 membered heterocyclyl containing 1, 2, or 3 heteroatoms selected from N, O, and S.

In some embodiments, R⁷ is

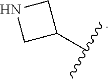 [SMILES: *C1CNC1]

In some embodiments, R⁷ is 5 to 6 membered heteroaryl containing 1, 2, or 3 heteroatoms selected from N, O, and S. In some embodiments, R⁷ is 5 to 6 membered heteroaryl containing 1, 2, or 3 heteroatoms selected from N, O, and S optionally substituted with one, two, or three R^{a} substituents independently selected from halogen, cyano, —N₃, —OR⁸, —NR⁹R¹⁰, —OP(═O)(OH)₂, C₃-C₈ carbocyclyl and phenyl. In some embodiments, R⁷ is 5 to 6 membered heteroaryl containing 1, 2, or 3 heteroatoms selected from N, O, and S optionally substituted with one, two, or three R^{a} substituents independently selected from halogen, cyano, —N₃, —OR⁸, —NR⁹R¹⁰, —OP(═O)(OH)₂, C₃-C₈ carbocyclyl and unsubstituted phenyl.

In some embodiments, R⁷ is 5 membered heteroaryl containing 1, 2, or 3 heteroatoms selected from N, O, and S. In some embodiments, R⁷ is 5 membered heteroaryl containing 1, 2, or 3 heteroatoms selected from N, O, and S optionally substituted with one, two, or three R^{a} substituents independently selected from halogen, cyano, —N₃, —OR⁸, —NR⁹R¹⁰, —OP(═O)(OH)₂, C₃-C₈ carbocyclyl and unsubstituted phenyl. In some embodiments, R⁷ is unsubstituted 5 membered heteroaryl containing 1, 2, or 3 heteroatoms selected from N, O, and S.

In some embodiments, R⁷ is 6 membered heteroaryl containing 1, 2, or 3 heteroatoms selected from N, O, and S. In some embodiments, R⁷ is 6 membered heteroaryl containing 1, 2, or 3 heteroatoms selected from N, O, and S optionally substituted with one, two, or three R^{a} substituents independently selected from halogen, cyano, —N₃, —OR⁸, —NR⁹R¹⁰, —OP(═O)(OH)₂, C₃-C₈ carbocyclyl and unsubstituted phenyl. In some embodiments, R⁷ is 6 membered heteroaryl containing 1, 2, or 3 heteroatoms selected from N, O, and S optionally substituted with one, two, or three R^{a} substituents independently selected from halogen, cyano, and —NR⁹R¹⁰. In some embodiments, R⁷ is unsubstituted 6 membered heteroaryl containing 1, 2, or 3 heteroatoms selected from N, O, and S.

In some embodiments, R⁷ is

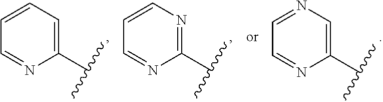

In some embodiments, R⁷ is

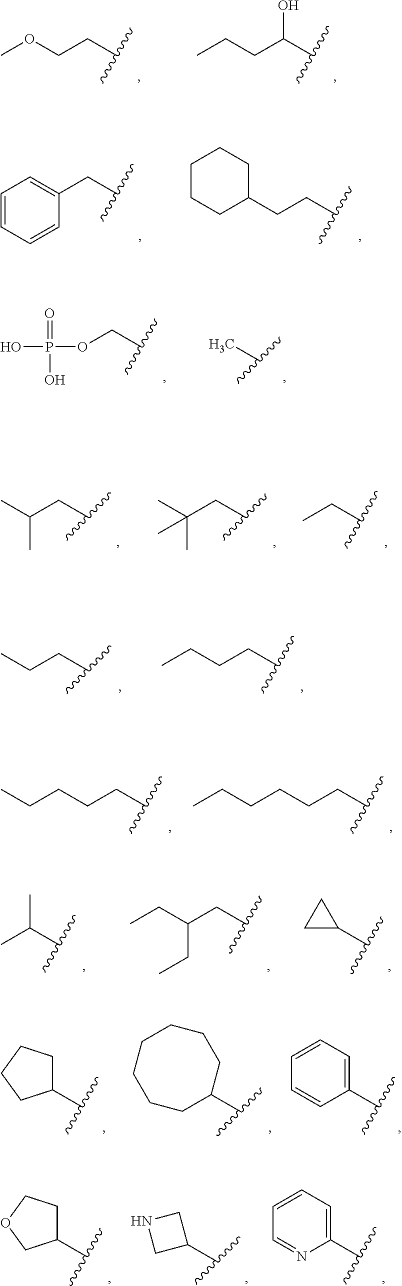

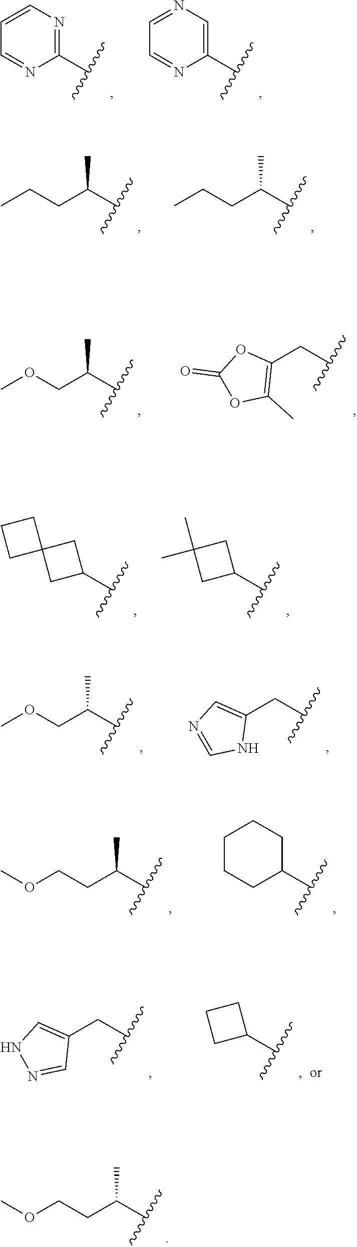

In some embodiments, R⁷ is

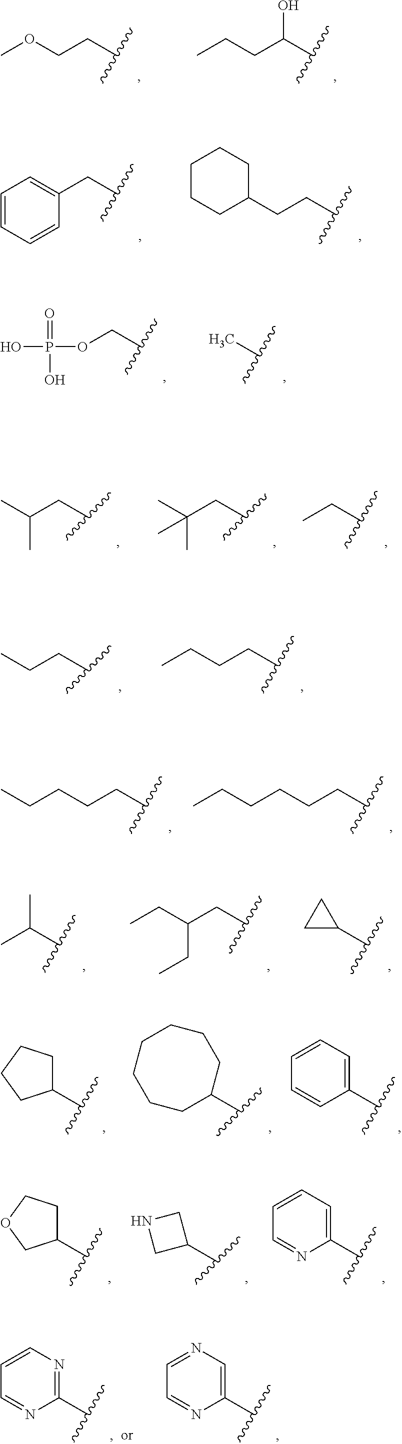

In some embodiments, R⁷ is

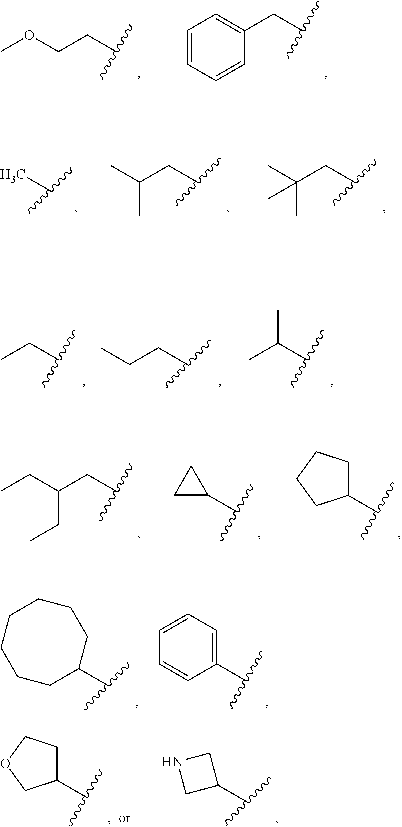

In some embodiments, R⁸ is H. In some embodiments, R⁸ is C₁-C₆ alkyl. In some embodiments, R⁸ is —CH₃. In some embodiments, R⁸ is C₁-C₆ haloalkyl. In some embodiments, R⁸ is C₃-C₆ cycloalkyl.

In some embodiments, R⁹ is H. In some embodiments, R⁹ is C₁-C₆ alkyl. In some embodiments, R⁹ is —CH₃. In some embodiments, R⁹ is C₁-C₆ haloalkyl. In some embodiments, R⁹ is C₃-C₆ cycloalkyl.

In some embodiments, R¹⁰ is H. In some embodiments, R¹⁰ is C₁-C₆ alkyl. In some embodiments, R¹⁰ is —CH₃. In some embodiments, R¹⁰ is C₁-C₆ haloalkyl. In some embodiments, R¹⁰ is C₃-C₆ cycloalkyl.

In some embodiments, Base is

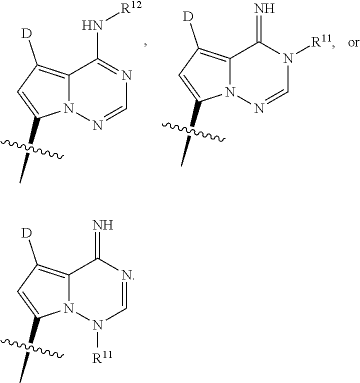

In some embodiments, Base is

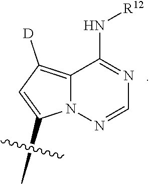

In some embodiments, Base is

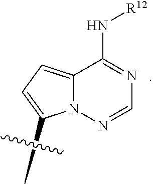

In some embodiments, Base is

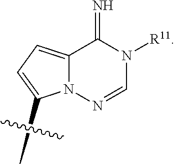

In some embodiments, Base is

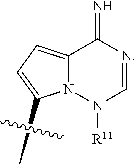

In some embodiments, Base is

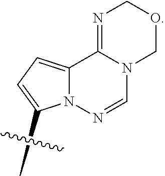

In some embodiments, R¹¹ is C₁-C₃ alkyl substituted with —OP(═O)(OH)(OR¹⁴). In some embodiments, R¹¹ is —(CH₂)OP(═O)(OH)(OR¹⁴).

In some embodiments, R¹⁴ is H.

In some embodiments, R¹⁴ is H or C₁-C₈ alkyl; wherein C₁-C₈ alkyl of R¹⁴ is optionally substituted with one, two or three substituents independently selected from halogen, cyano, and phenyl.

In some embodiments, R¹⁴ is C₁-C₈ alkyl optionally substituted with one, two or three substituents independently selected from halogen, cyano, and phenyl. In some embodiments, R¹⁴ is C₁-C₃ alkyl optionally substituted with one, two or three substituents independently selected from halogen, cyano, and phenyl. In some embodiments, R¹⁴ is C₁-C₃ alkyl substituted with one phenyl. In some embodiments, R¹⁴ is

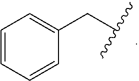 [SMILES: *Cc1ccccc1]

In some embodiments, R¹¹ is —(CH₂)OP(═O)(OH)₂. In some embodiments, R¹¹ is

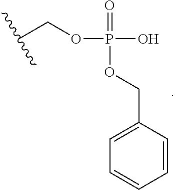 [SMILES: *COP(=O)(O)OCc1ccccc1]

In some embodiments, R¹² is H. In some embodiments, R¹² is C₁-C₆ alkyl. In some embodiments, R¹² is —C(═O)R¹³. In some embodiments, R¹² is —C(═O)(CH₂)₂CH₃. In some embodiments, R¹² is —C(═O)OR¹³. In some embodiments, R¹² is —C(═O)OCH₂CH(CH₃)₂. In some embodiments, R¹² is —C(═O)OCH₂CH(CH₃)₂ or —C(═O)(CH₂)₂CH₃. In some embodiments, R¹² is

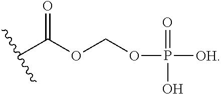 [SMILES: *C(=O)OCOP(=O)(O)O]

In some embodiments, R¹³ is H. In some embodiments, R¹³ is C₁-C₁₀ alkyl. In some embodiments, R¹³ is C₁-C₈ alkyl. In some embodiments, R¹³ is C₁-C₈ alkyl optionally substituted with one, two, or three R^{c} substituents independently selected from halogen, cyano, —OP(═O)(OH)(OR¹⁴) and phenyl. In some embodiments, R¹³ is C₁-C₈ alkyl optionally substituted with one, two, or three R^{c} substituents independently selected from halogen, cyano, and phenyl. In some embodiments, R¹³ is C₁-C₈ alkyl optionally substituted with one, two, or three R^{c} substituents independently selected from halogen, cyano, and unsubstituted phenyl. In some embodiments, R¹³ is C₁-C₈ alkyl. In some embodiments, R¹³ is —CH₃, —CH₂CH₃, —(CH₂)₂CH₃, —CH(CH₃)₂, —(CH₂)₃CH₃, or —C(CH₃)₃. In some embodiments, R¹³ is —CH₂CH(CH₃)₂ or —(CH₂)₂CH₃. In some embodiments, R¹³ is —(CH₂)OP(═O)(OH)₂. In some embodiments, R¹³ is C₆-C₁₀ aryl. In some embodiments, R¹³ is phenyl. In some embodiments, R¹³ is —O—C₆-C₁₀ aryl. In some embodiments, R¹³ is —O-phenyl. In some embodiments, R¹³ is —O—C₁-C₁₀ alkyl. In some embodiments, R¹³ is —O—CH₃, —O—CH₂CH₃, —O—(CH₂)₂CH₃, —O—(CH₂)₄CH₃, or —O—(CH₂)₆CH₃.

In some embodiments, R¹³ is

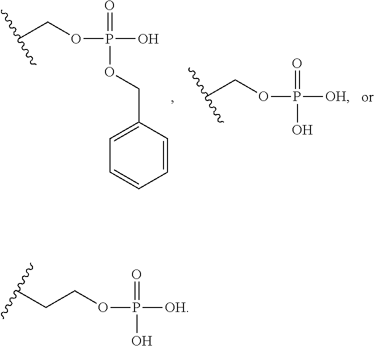 [SMILES: *COP(=O)(O)OCc1ccccc1 ; *COP(=O)(O)O ; *CCOP(=O)(O)O]

In some embodiments, Base is

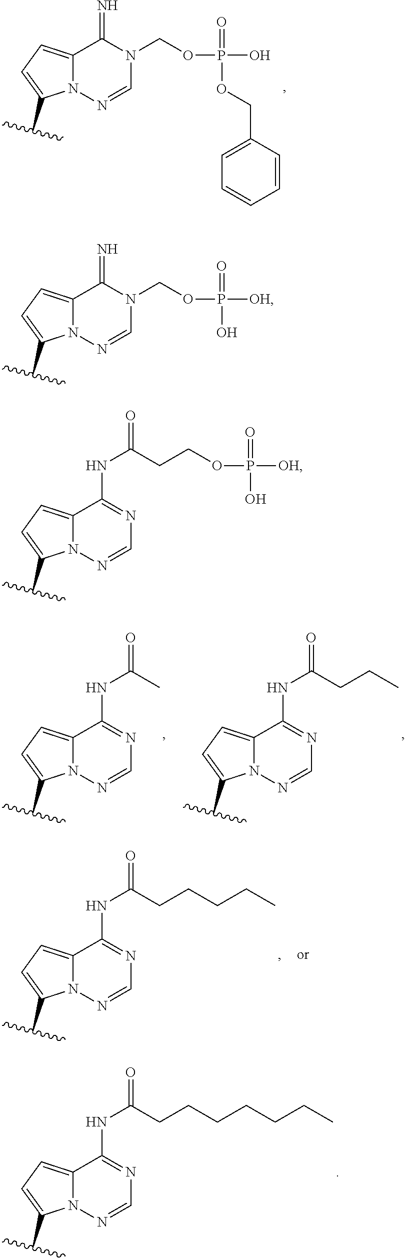

In some embodiments, the method comprises administering to the patient a compound of Formula A or a prodrug thereof, or a pharmaceutically acceptable salt thereof, wherein
R¹ is —OH, —OC(═O)R⁴, or —OC(═O)OR⁴;
R² is —OH, —OC(═O)R⁵, or —OC(═O)OR⁵; or
R¹ and R² are taken together to form —OC(═O)O—; and
R⁴, and R⁵ are each independently unsubstituted C₁-C₈ alkyl, unsubstituted C₃-C₈ carbocyclyl, unsubstituted C₆-C₁₀ aryl, unsubstituted 4 to 6 membered heterocyclyl containing 1, 2, or 3 heteroatoms selected from N, O, and S, or unsubstituted 5 to 6 membered heteroaryl containing 1, 2, or 3 heteroatoms selected from N, O, and S.

In some embodiments, Base is

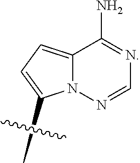

In some embodiments, R⁴ and R⁵ are each independently unsubstituted C₁-C₈ alkyl. In some embodiments, R⁴ and R⁵ are each independently unsubstituted C₁-C₆ alkyl. In some embodiments, R¹ is —OH or —OC(═O)R⁴ and R² is —OH or —OC(═O)R⁵. In some embodiments, R¹ and R² are each independently —OH, OC(═O)CH(CH₃)₂, or OC(═O)CH₃. In some embodiments, R¹ and R² are taken together to form —OC(═O)O—.

In some embodiments, the method comprises administering to the patient a deuterated compound of Formula A which has Formula A1

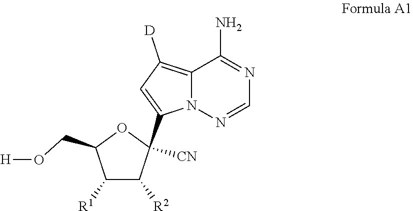

or a prodrug thereof, or a pharmaceutically acceptable salt thereof, wherein
R¹ is —OH, —OC(═O)R⁴, or —OC(═O)OR⁴;
R² is —OH, —OC(═O)R⁵, or —OC(═O)OR⁵; or
R¹ and R² are taken together to form —OC(═O)O—; and
R⁴, and R⁵ are each independently unsubstituted C₁-C₈ alkyl, unsubstituted C₃-C₈ carbocyclyl, unsubstituted C₆-C₁₀ aryl, unsubstituted 4 to 6 membered heterocyclyl containing 1, 2, or 3 heteroatoms selected from N, O, and S, or unsubstituted 5 to 6 membered heteroaryl containing 1, 2, or 3 heteroatoms selected from N, O, and S.

In some embodiments, R⁴ and R⁵ are each independently unsubstituted C₁-C₈ alkyl. In some embodiments, R⁴ and R⁵ are each independently unsubstituted C₁-C₆ alkyl. In some embodiments, R¹ is —OH or —OC(═O)R⁴ and R² is —OH or —OC(═O)R⁵. In some embodiments, R¹ and R² are each independently —OH, OC(═O)CH(CH₃)₂, or OC(═O)CH₃. In some embodiments, R¹ and R² are taken together to form —OC(═O)O—.

In some embodiments, the method comprises administering to the patient a deuterated compound of Formula A which has Formula A2

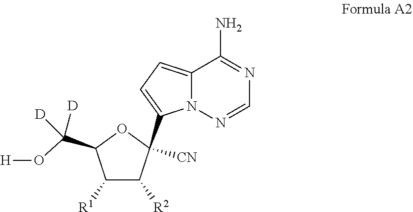

or a prodrug thereof, or a pharmaceutically acceptable salt thereof, wherein
R¹ is —OH, —OC(═O)R⁴, or —OC(═O)OR⁴;
R² is —OH, —OC(═O)R⁵, or —OC(═O)OR⁵; or
R¹ and R² are taken together to form —OC(═O)O—; and
R⁴, and R⁵ are each independently unsubstituted C₁-C₈ alkyl, unsubstituted C₃-C₈ carbocyclyl, unsubstituted C₆-C₁₀ aryl, unsubstituted 4 to 6 membered heterocyclyl containing 1, 2, or 3 heteroatoms selected from N, O, and S, or unsubstituted 5 to 6 membered heteroaryl containing 1, 2, or 3 heteroatoms selected from N, O, and S.

In some embodiments, R⁴ and R⁵ are each independently unsubstituted C₁-C₈ alkyl. In some embodiments, R⁴ and R⁵ are each independently unsubstituted C₁-C₆ alkyl. In some embodiments, R¹ is —OH or —OC(═O)R⁴ and R² is —OH or —OC(═O)R⁵. In some embodiments, R¹ and R² are each independently —OH, OC(═O)CH(CH₃)₂, or OC(═O)CH₃. In some embodiments, R¹ and R² are taken together to form —OC(═O)O—.

In some embodiments, the method comprises administering to the patient a compound of Formula I, or a pharmaceutically acceptable salt thereof, wherein
R¹ is —OH, —OC(═O)R⁴, or —OC(═O)OR⁴;
R² is —OH, —OC(═O)R⁵, or —OC(═O)OR⁵; or
R¹ and R² are taken together to form —OC(═O)O—;
R³ is —C(═O)OR⁷ or —C(═O)R⁷; and
R⁴, R⁵, and R⁷ are each independently unsubstituted C₁-C₈ alkyl, unsubstituted C₃-C₈ carbocyclyl, unsubstituted C₆-C₁₀ aryl, unsubstituted 4 to 6 membered heterocyclyl containing 1, 2, or 3 heteroatoms selected from N, O, and S, or unsubstituted 5 to 6 membered heteroaryl containing 1, 2, or 3 heteroatoms selected from N, O, and S.

In some embodiments, Base is

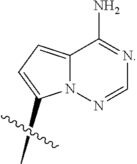

In some embodiments, R⁴, R⁵, and R⁷ are each independently unsubstituted C₁-C₈ alkyl. In some embodiments, R⁴, R⁵, and R⁷ are each independently unsubstituted C₁-C₆ alkyl. In some embodiments, R¹ is —OH or —OC(═O)R⁴, R² is —OH or —OC(═O)R⁵, and R³ is —C(═O)R⁷. In some embodiments, R¹ and R² are each independently —OH, OC(═O)CH(CH₃)₂, or OC(═O)CH₃, and R⁷ is —CH(CH₃)₂ or —CH₃. In some embodiments, R¹ and R² are taken together to form —OC(═O)O—, and R⁷ is —CH(CH₃)₂ or —CH₃.

In some embodiments, the method comprises administering to the patient a deuterated compound of Formula I which has Formula Ia

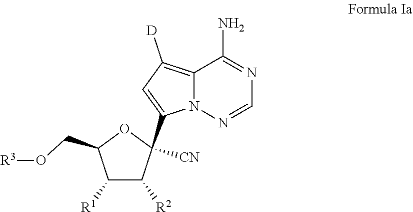

or a pharmaceutically acceptable salt thereof, wherein
R¹ is —OH, —OC(═O)R⁴, or —OC(═O)OR⁴;
R² is —OH, —OC(═O)R⁵, or —OC(═O)OR⁵; or
R¹ and R² are taken together to form —OC(═O)O—;
R³ is —C(═O)OR⁷ or —C(═O)R⁷; and
R⁴, R⁵, and R⁷ are each independently unsubstituted C₁-C₈ alkyl, unsubstituted C₃-C₈ carbocyclyl, unsubstituted C₆-C₁₀ aryl, unsubstituted 4 to 6 membered heterocyclyl containing 1, 2, or 3 heteroatoms selected from N, O, and S, or unsubstituted 5 to 6 membered heteroaryl containing 1, 2, or 3 heteroatoms selected from N, O, and S.

In some embodiments, R⁴, R⁵, and R⁷ are each independently unsubstituted C₁-C₈ alkyl. In some embodiments, R⁴, R⁵, and R⁷ are each independently unsubstituted C₁-C₆ alkyl. In some embodiments, R¹ is —OH or —OC(═O)R⁴, R² is —OH or —OC(═O)R⁵, and R³ is —C(═O)R⁷. In some embodiments, R¹ and R² are each independently —OH, OC(═O)CH(CH₃)₂, or OC(═O)CH₃, and R⁷ is —CH(CH₃)₂ or —CH₃. In some embodiments, R¹ and R² are taken together to form —OC(═O)O—, and R⁷ is —CH(CH₃)₂ or —CH₃.

In some embodiments, the method comprises administering to the patient a deuterated compound of Formula I which has Formula Ib

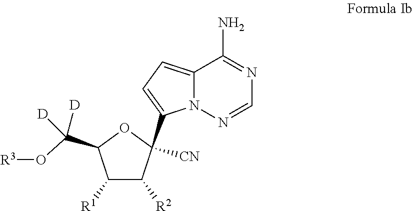

or a pharmaceutically acceptable salt thereof, wherein
R¹ is —OH, —OC(═O)R⁴, or —OC(═O)OR⁴;
R² is —OH, —OC(═O)R⁵, or —OC(═O)OR⁵; or
R¹ and R² are taken together to form —OC(═O)O—;
R³ is —C(═O)OR⁷ or —C(═O)R⁷; and
R⁴, R⁵, and R⁷ are each independently unsubstituted C₁-C₈ alkyl, unsubstituted C₃-C₈ carbocyclyl, unsubstituted C₆-C₁₀ aryl, unsubstituted 4 to 6 membered heterocyclyl containing 1, 2, or 3 heteroatoms selected from N, O, and S, or unsubstituted 5 to 6 membered heteroaryl containing 1, 2, or 3 heteroatoms selected from N, O, and S.

In some embodiments, R⁴, R⁵, and R⁷ are each independently unsubstituted C₁-C₈ alkyl. In some embodiments, R⁴, R⁵, and R⁷ are each independently unsubstituted C₁-C₆ alkyl. In some embodiments, R¹ is —OH or —OC(═O)R⁴, R² is —OH or —OC(═O)R⁵, and R³ is —C(═O)R⁷. In some embodiments, R¹ and R² are each independently —OH, OC(═O)CH(CH₃)₂, or OC(═O)CH₃, and R⁷ is —CH(CH₃)₂ or —CH₃. In some embodiments, R¹ and R² are taken together to form —OC(═O)O—, and R⁷ is —CH(CH₃)₂ or —CH₃.

In some embodiments, the compound of Formula A or the prodrug of the compound of Formula A is a compound of Table 1, or a pharmaceutically acceptable salt thereof.

TABLE 1

| | |
|---|---|
| Compound 1 | 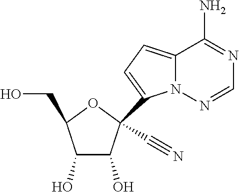 [SMILES: N#C[C@@]1(c2ccc3c(N)ncnn23)O[C@H](CO)[C@@H](O)[C@H]1O] |
| Compound 2 | 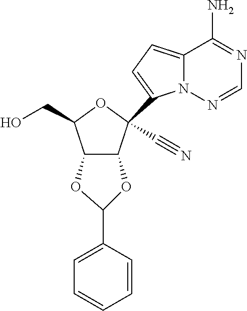 |
| Compound 3 | 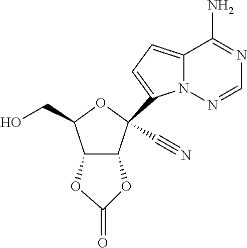 [SMILES: N#C[C@@]1(c2ccc3c(N)ncnn23)O[C@H](CO)[C@H]2OC(=O)O[C@H]21] |

In some embodiments, the compound of Formula I is a compound of Table 2, or a pharmaceutically acceptable salt thereof.

TABLE 2

| | |
|---|---|
| Compound 4 | 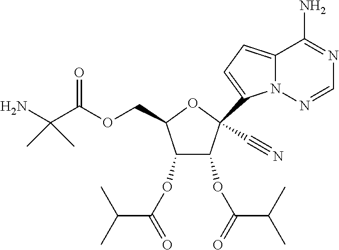 [SMILES: CC(C)C(=O)O[C@H]1[C@@H](OC(=O)C(C)C)[C@](C#N)(c2ccc3c(N)ncnn23)O[C@@H]1COC(=O)C(C)(C)N] |
| Compound 5 | 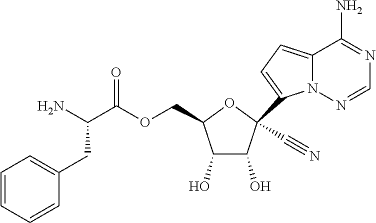 [SMILES: N#C[C@@]1(c2ccc3c(N)ncnn23)O[C@H](COC(=O)[C@@H](N)Cc2ccccc2)[C@@H](O)[C@H]1O] |
| Compound 6 | 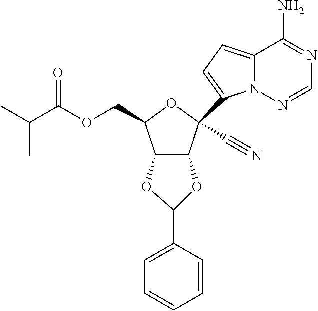 |
| Compound 7 | 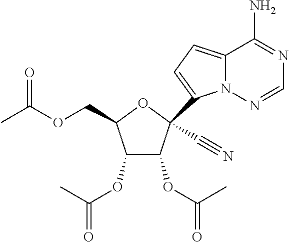 [SMILES: CC(=O)OC[C@H]1O[C@@](C#N)(c2ccc3c(N)ncnn23)[C@H](OC(C)=O)[C@@H]1OC(C)=O] |
| Compound 8 | 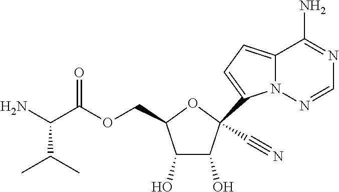 [SMILES: CC(C)[C@H](N)C(=O)OC[C@H]1O[C@@](C#N)(c2ccc3c(N)ncnn23)[C@H](O)[C@@H]1O] |
| Compound 9 | 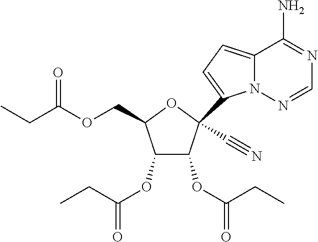 [SMILES: CCC(=O)OC[C@H]1O[C@@](C#N)(c2ccc3c(N)ncnn23)[C@H](OC(=O)CC)[C@@H]1OC(=O)CC] |
| Compound 10 | 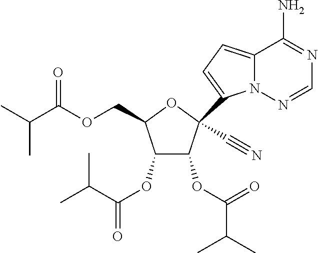 [SMILES: CC(C)C(=O)OC[C@H]1O[C@@](C#N)(c2ccc3c(N)ncnn23)[C@H](OC(=O)C(C)C)[C@@H]1OC(=O)C(C)C] |
| Compound 11 | 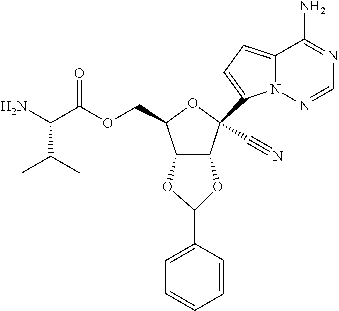 |
| Compound 12 | 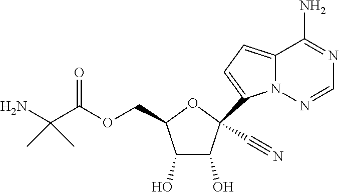 [SMILES: CC(C)(N)C(=O)OC[C@H]1O[C@@](C#N)(c2ccc3c(N)ncnn23)[C@H](O)[C@@H]1O] |
| Compound 13 | 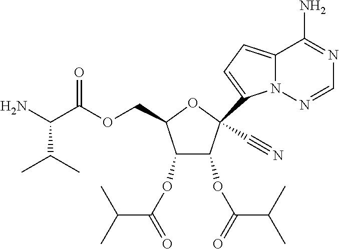 [SMILES: CC(C)C(=O)O[C@H]1[C@@H](OC(=O)C(C)C)[C@](C#N)(c2ccc3c(N)ncnn23)O[C@@H]1COC(=O)[C@@H](N)C(C)C] |
| Compound 14 | 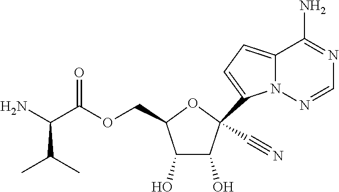 [SMILES: CC(C)[C@@H](N)C(=O)OC[C@H]1O[C@@](C#N)(c2ccc3c(N)ncnn23)[C@H](O)[C@@H]1O] |
| Compound 15 | 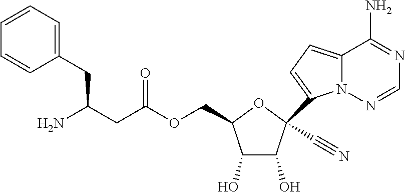 [SMILES: N#C[C@@]1(c2ccc3c(N)ncnn23)O[C@H](COC(=O)C[C@@H](N)Cc2ccccc2)[C@@H](O)[C@H]1O] |
| Compound 16 | 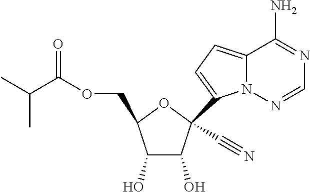 [SMILES: CC(C)C(=O)OC[C@H]1O[C@@](C#N)(c2ccc3c(N)ncnn23)[C@H](O)[C@@H]1O] |
| Compound 17 | 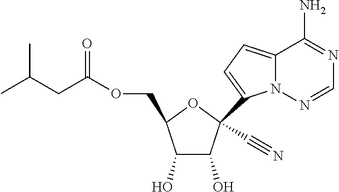 [SMILES: CC(C)CC(=O)OC[C@H]1O[C@@](C#N)(c2ccc3c(N)ncnn23)[C@H](O)[C@@H]1O] |
| Compound 18 | 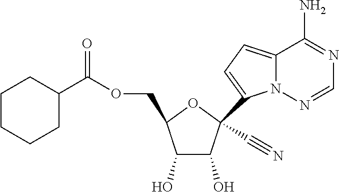 [SMILES: N#C[C@@]1(c2ccc3c(N)ncnn23)O[C@H](COC(=O)C2CCCCC2)[C@@H](O)[C@H]1O] |
| Compound 19 | 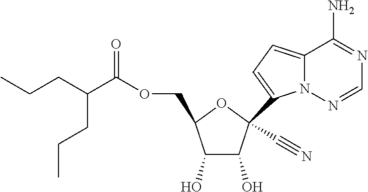 [SMILES: CCCC(CCC)C(=O)OC[C@H]1O[C@@](C#N)(c2ccc3c(N)ncnn23)[C@H](O)[C@@H]1O] |
| Compound 20 | 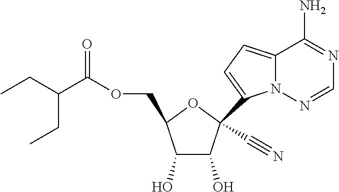 [SMILES: CCC(CC)C(=O)OC[C@H]1O[C@@](C#N)(c2ccc3c(N)ncnn23)[C@H](O)[C@@H]1O] |
| Compound 21 | 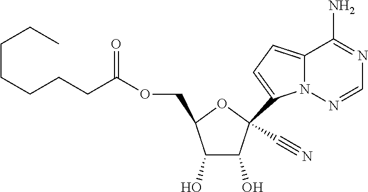 [SMILES: CCCCCCCC(=O)OC[C@H]1O[C@@](C#N)(c2ccc3c(N)ncnn23)[C@H](O)[C@@H]1O] |
| Compound 22 | 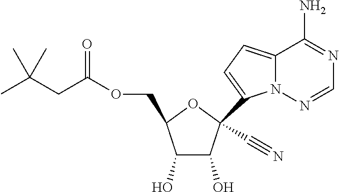 [SMILES: CC(C)(C)CC(=O)OC[C@H]1O[C@@](C#N)(c2ccc3c(N)ncnn23)[C@H](O)[C@@H]1O] |
| Compound 23 | 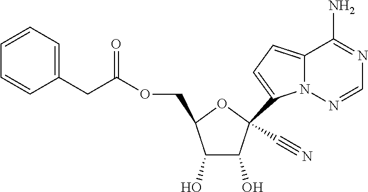 [SMILES: N#C[C@@]1(c2ccc3c(N)ncnn23)O[C@H](COC(=O)Cc2ccccc2)[C@@H](O)[C@H]1O] |
| Compound 24 | 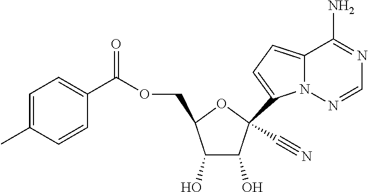 [SMILES: Cc1ccc(C(=O)OC[C@H]2O[C@@](C#N)(c3ccc4c(N)ncnn34)[C@H](O)[C@@H]2O)cc1] |
| Compound 25 | 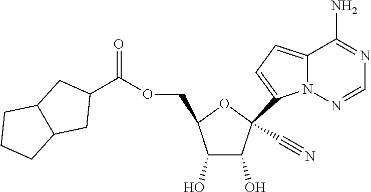 [SMILES: N#C[C@@]1(c2ccc3c(N)ncnn23)O[C@H](COC(=O)C2CC3CCCC3C2)[C@@H](O)[C@H]1O] |
| Compound 26 | 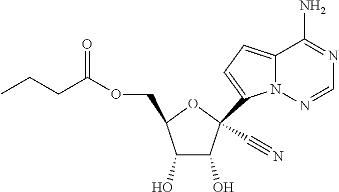 [SMILES: CCCC(=O)OC[C@H]1O[C@@](C#N)(c2ccc3c(N)ncnn23)[C@H](O)[C@@H]1O] |
| Compound 27 | 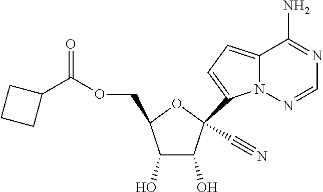 [SMILES: N#C[C@@]1(c2ccc3c(N)ncnn23)O[C@H](COC(=O)C2CCC2)[C@@H](O)[C@H]1O] |
| Compound 28 | 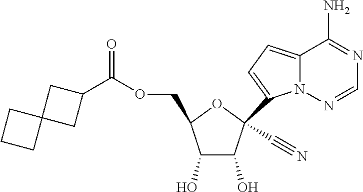 [SMILES: N#C[C@@]1(c2ccc3c(N)ncnn23)O[C@H](COC(=O)C2CC3(CCC3)C2)[C@@H](O)[C@H]1O] |
| Compound 29 | 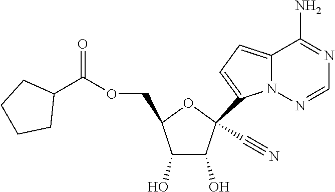 [SMILES: N#C[C@@]1(c2ccc3c(N)ncnn23)O[C@H](COC(=O)C2CCCC2)[C@@H](O)[C@H]1O] |
| Compound 30 | 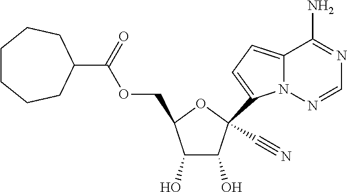 [SMILES: N#C[C@@]1(c2ccc3c(N)ncnn23)O[C@H](COC(=O)C2CCCCCC2)[C@@H](O)[C@H]1O] |
| Compound 31 | 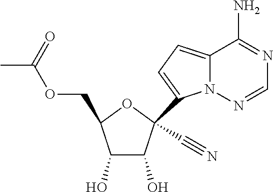 [SMILES: CC(=O)OC[C@H]1O[C@@](C#N)(c2ccc3c(N)ncnn23)[C@H](O)[C@@H]1O] |
| Compound 32 | 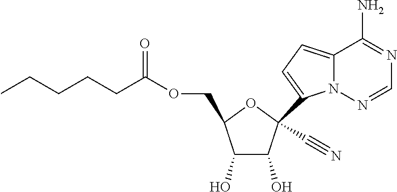 [SMILES: CCCCCC(=O)OC[C@H]1O[C@@](C#N)(c2ccc3c(N)ncnn23)[C@H](O)[C@@H]1O] |
| Compound 33 | 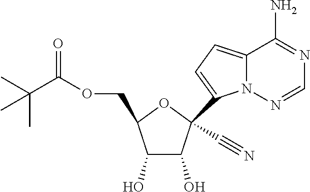 [SMILES: CC(C)(C)C(=O)OC[C@H]1O[C@@](C#N)(c2ccc3c(N)ncnn23)[C@H](O)[C@@H]1O] |
| Compound 34 | 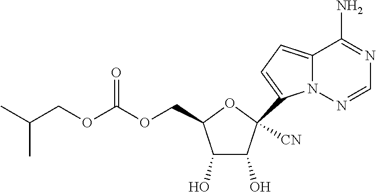 [SMILES: CC(C)COC(=O)OC[C@H]1O[C@@](C#N)(c2ccc3c(N)ncnn23)[C@H](O)[C@@H]1O] |
| Compound 35 | 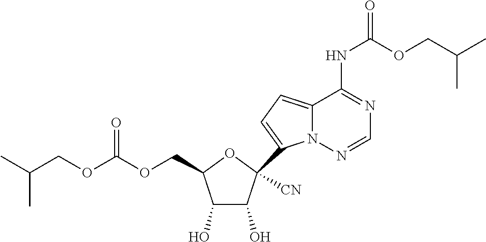 [SMILES: CC(C)COC(=O)Nc1ncnn2c([C@]3(C#N)O[C@H](COC(=O)OCC(C)C)[C@@H](O)[C@H]3O)ccc12] |
| Compound 36 | 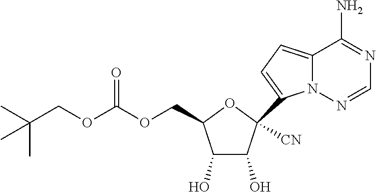 [SMILES: CC(C)(C)COC(=O)OC[C@H]1O[C@@](C#N)(c2ccc3c(N)ncnn23)[C@H](O)[C@@H]1O] |
| Compound 37 | 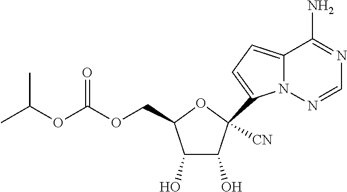 [SMILES: CC(C)OC(=O)OC[C@H]1O[C@@](C#N)(c2ccc3c(N)ncnn23)[C@H](O)[C@@H]1O] |
| Compound 38 | 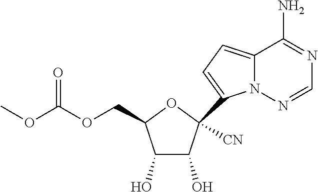 [SMILES: COC(=O)OC[C@H]1O[C@@](C#N)(c2ccc3c(N)ncnn23)[C@H](O)[C@@H]1O] |
| Compound 39 | 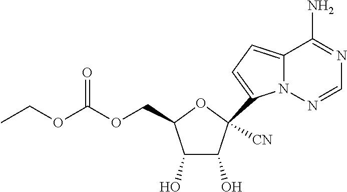 [SMILES: CCOC(=O)OC[C@H]1O[C@@](C#N)(c2ccc3c(N)ncnn23)[C@H](O)[C@@H]1O] |
| Compound 40 | 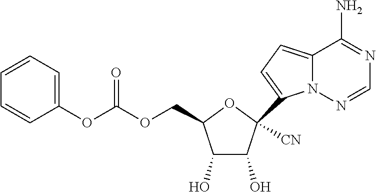 [SMILES: N#C[C@@]1(c2ccc3c(N)ncnn23)O[C@H](COC(=O)Oc2ccccc2)[C@@H](O)[C@H]1O] |
| Compound 41 | 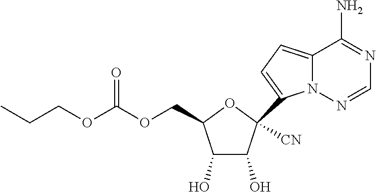 [SMILES: CCCOC(=O)OC[C@H]1O[C@@](C#N)(c2ccc3c(N)ncnn23)[C@H](O)[C@@H]1O] |
| Compound 42 | 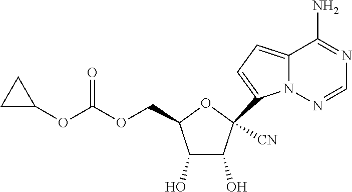 [SMILES: N#C[C@@]1(c2ccc3c(N)ncnn23)O[C@H](COC(=O)OC2CC2)[C@@H](O)[C@H]1O] |
| Compound 43 | 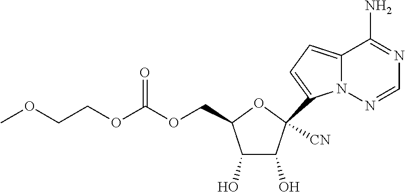 [SMILES: COCCOC(=O)OC[C@H]1O[C@@](C#N)(c2ccc3c(N)ncnn23)[C@H](O)[C@@H]1O] |
| Compound 44 | 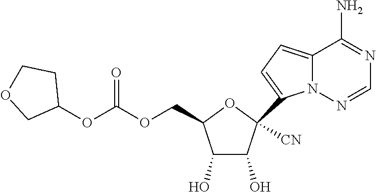 [SMILES: N#C[C@@]1(c2ccc3c(N)ncnn23)O[C@H](COC(=O)OC2CCOC2)[C@@H](O)[C@H]1O] |
| Compound 45 | 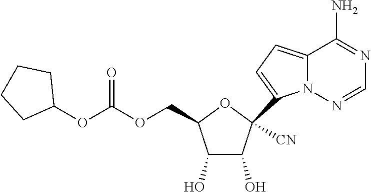 [SMILES: N#C[C@@]1(c2ccc3c(N)ncnn23)O[C@H](COC(=O)OC2CCCC2)[C@@H](O)[C@H]1O] |
| Compound 46 | 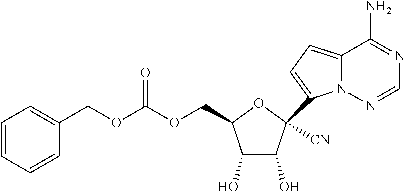 [SMILES: N#C[C@@]1(c2ccc3c(N)ncnn23)O[C@H](COC(=O)OCc2ccccc2)[C@@H](O)[C@H]1O] |
| Compound 47 | 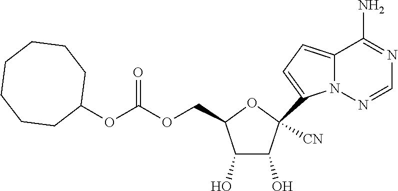 [SMILES: N#C[C@@]1(c2ccc3c(N)ncnn23)O[C@H](COC(=O)OC2CCCCCCC2)[C@@H](O)[C@H]1O] |
| Compound 48 | 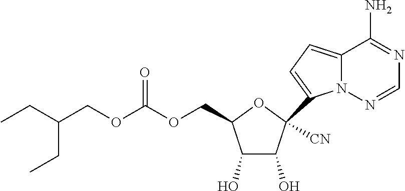 [SMILES: CCC(CC)COC(=O)OC[C@H]1O[C@@](C#N)(c2ccc3c(N)ncnn23)[C@H](O)[C@@H]1O] |
| Compound 49 | 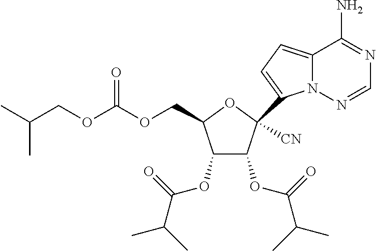 [SMILES: CC(C)COC(=O)OC[C@H]1O[C@@](C#N)(c2ccc3c(N)ncnn23)[C@H](OC(=O)C(C)C)[C@@H]1OC(=O)C(C)C] |
| Compound 50 | 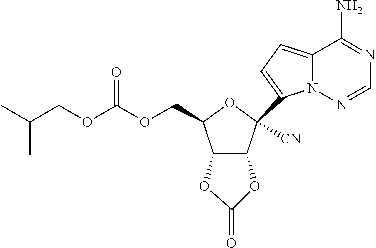 [SMILES: CC(C)COC(=O)OC[C@H]1O[C@@](C#N)(c2ccc3c(N)ncnn23)[C@@H]2OC(=O)O[C@@H]21] |
| Compound 51 | 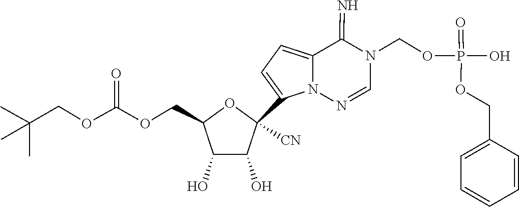 [SMILES: CC(C)(C)COC(=O)OC[C@H]1O[C@@](C#N)(c2ccc3c(=N)n(COP(=O)(O)OCc4ccccc4)cnn23)[C@H](O)[C@@H]1O] |
| Compound 52 | 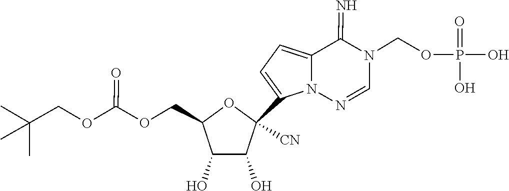 [SMILES: CC(C)(C)COC(=O)OC[C@H]1O[C@@](C#N)(c2ccc3c(=N)n(COP(=O)(O)O)cnn23)[C@H](O)[C@@H]1O] |
| Compound 53 | 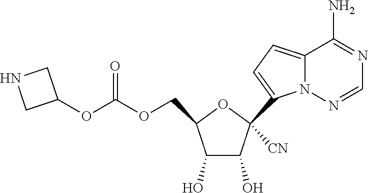 [SMILES: N#C[C@@]1(c2ccc3c(N)ncnn23)O[C@H](COC(=O)OC2CNC2)[C@@H](O)[C@H]1O] |
| Compound 54 | 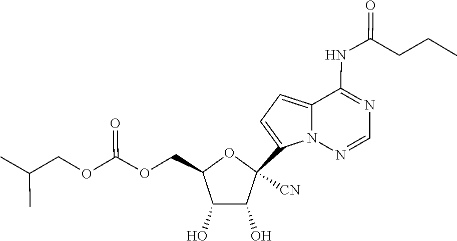 [SMILES: CCCC(=O)Nc1ncnn2c([C@]3(C#N)O[C@H](COC(=O)OCC(C)C)[C@@H](O)[C@H]3O)ccc12] |
| Compound 55 | 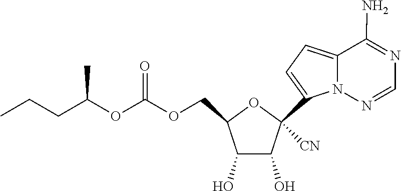 [SMILES: CCC[C@@H](C)OC(=O)OC[C@H]1O[C@@](C#N)(c2ccc3c(N)ncnn23)[C@H](O)[C@@H]1O] |
| Compound 56 | 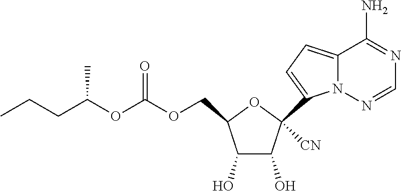 [SMILES: CCC[C@H](C)OC(=O)OC[C@H]1O[C@@](C#N)(c2ccc3c(N)ncnn23)[C@H](O)[C@@H]1O] |
| Compound 57 | 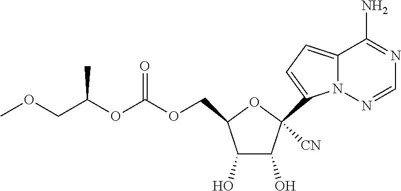 [SMILES: COC[C@@H](C)OC(=O)OC[C@H]1O[C@@](C#N)(c2ccc3c(N)ncnn23)[C@H](O)[C@@H]1O] |
| Compound 58 | 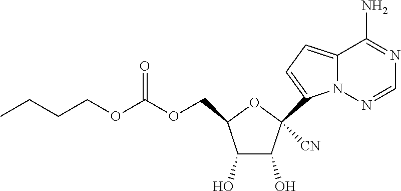 [SMILES: CCCCOC(=O)OC[C@H]1O[C@@](C#N)(c2ccc3c(N)ncnn23)[C@H](O)[C@@H]1O] |
| Compound 59 | 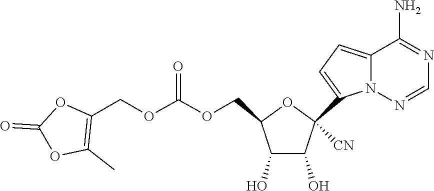 [SMILES: Cc1oc(=O)oc1COC(=O)OC[C@H]1O[C@@](C#N)(c2ccc3c(N)ncnn23)[C@H](O)[C@@H]1O] |
| Compound 60 | 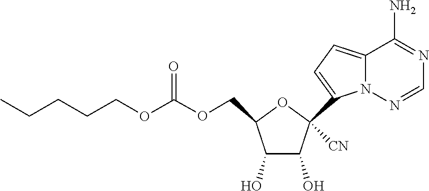 [SMILES: CCCCCOC(=O)OC[C@H]1O[C@@](C#N)(c2ccc3c(N)ncnn23)[C@H](O)[C@@H]1O] |
| Compound 61 | 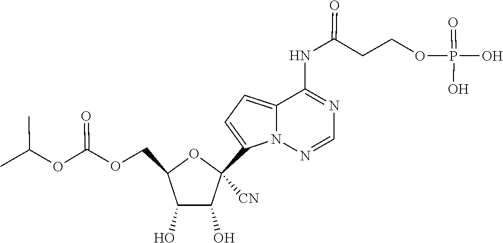 [SMILES: CC(C)OC(=O)OC[C@H]1O[C@@](C#N)(c2ccc3c(NC(=O)CCOP(=O)(O)O)ncnn23)[C@H](O)[C@@H]1O] |
| Compound 62 | 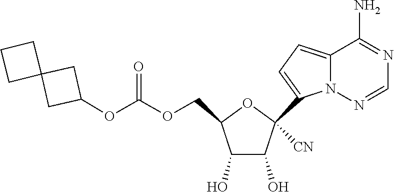 [SMILES: N#C[C@@]1(c2ccc3c(N)ncnn23)O[C@H](COC(=O)OC2CC3(CCC3)C2)[C@@H](O)[C@H]1O] |
| Compound 63 | 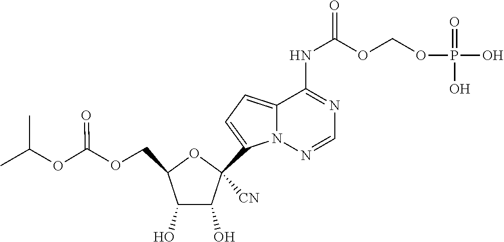 [SMILES: CC(C)OC(=O)OC[C@H]1O[C@@](C#N)(c2ccc3c(NC(=O)OCOP(=O)(O)O)ncnn23)[C@H](O)[C@@H]1O] |
| Compound 64 | 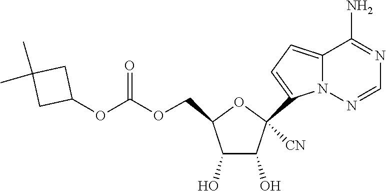 [SMILES: CC1(C)CC(OC(=O)OC[C@H]2O[C@@](C#N)(c3ccc4c(N)ncnn34)[C@H](O)[C@@H]2O)C1] |
| Compound 65 | 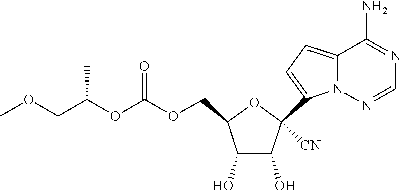 [SMILES: COC[C@H](C)OC(=O)OC[C@H]1O[C@@](C#N)(c2ccc3c(N)ncnn23)[C@H](O)[C@@H]1O] |
| Compound 66 | 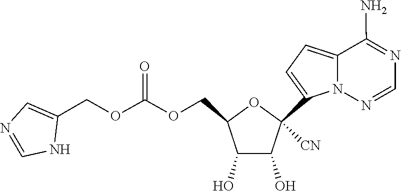 [SMILES: N#C[C@@]1(c2ccc3c(N)ncnn23)O[C@H](COC(=O)OCc2cnc[nH]2)[C@@H](O)[C@H]1O] |
| Compound 67 | 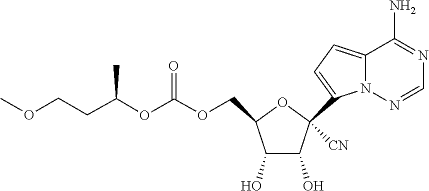 [SMILES: COCC[C@@H](C)OC(=O)OC[C@H]1O[C@@](C#N)(c2ccc3c(N)ncnn23)[C@H](O)[C@@H]1O] |
| Compound 68 | 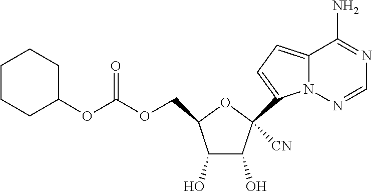 [SMILES: N#C[C@@]1(c2ccc3c(N)ncnn23)O[C@H](COC(=O)OC2CCCCC2)[C@@H](O)[C@H]1O] |
| Compound 69 | 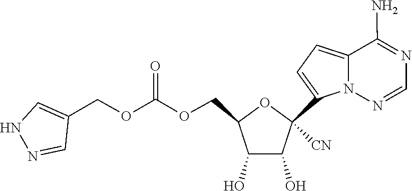 [SMILES: N#C[C@@]1(c2ccc3c(N)ncnn23)O[C@H](COC(=O)OCc2cn[nH]c2)[C@@H](O)[C@H]1O] |
| Compound 70 | 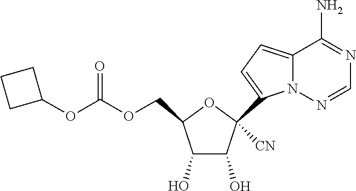 [SMILES: N#C[C@@]1(c2ccc3c(N)ncnn23)O[C@H](COC(=O)OC2CCC2)[C@@H](O)[C@H]1O] |
| Compound 71 | 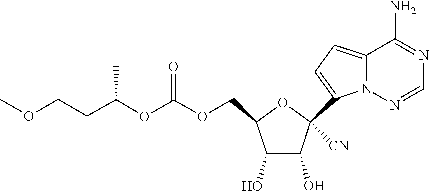 [SMILES: COCC[C@H](C)OC(=O)OC[C@H]1O[C@@](C#N)(c2ccc3c(N)ncnn23)[C@H](O)[C@@H]1O] |
| Compound 72 | 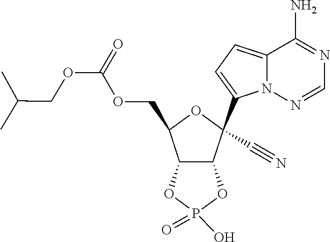 |
| Compound 73 | 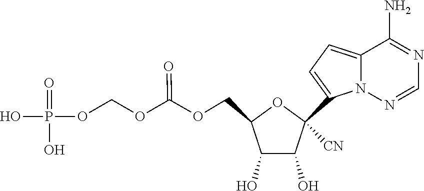 [SMILES: N#C[C@@]1(c2ccc3c(N)ncnn23)O[C@H](COC(=O)OCOP(=O)(O)O)[C@@H](O)[C@H]1O] |
| Compound 74 | 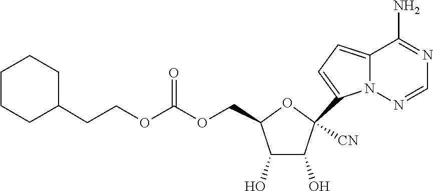 [SMILES: N#C[C@@]1(c2ccc3c(N)ncnn23)O[C@H](COC(=O)OCCC2CCCCC2)[C@@H](O)[C@H]1O] |
| Compound 75 | 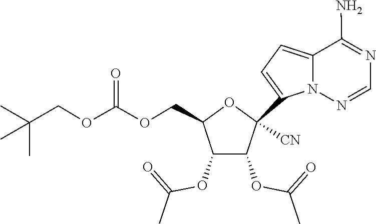 [SMILES: CC(=O)O[C@H]1[C@@H](OC(C)=O)[C@](C#N)(c2ccc3c(N)ncnn23)O[C@@H]1COC(=O)OCC(C)(C)C] |
| Compound 76 | 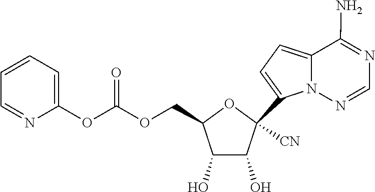 [SMILES: N#C[C@@]1(c2ccc3c(N)ncnn23)O[C@H](COC(=O)Oc2ccccn2)[C@@H](O)[C@H]1O] |
| Compound 77 | 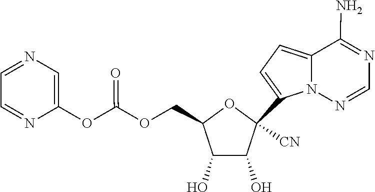 [SMILES: N#C[C@@]1(c2ccc3c(N)ncnn23)O[C@H](COC(=O)Oc2cnccn2)[C@@H](O)[C@H]1O] |
| Compound 78 | 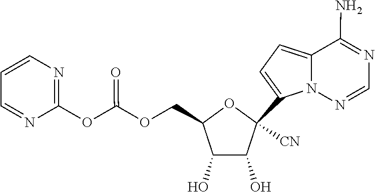 [SMILES: N#C[C@@]1(c2ccc3c(N)ncnn23)O[C@H](COC(=O)Oc2ncccn2)[C@@H](O)[C@H]1O] |
| Compound 79 | 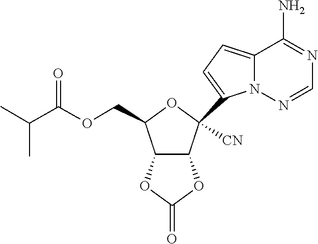 [SMILES: CC(C)C(=O)OC[C@H]1O[C@@](C#N)(c2ccc3c(N)ncnn23)[C@@H]2OC(=O)O[C@@H]21] |
| Compound 80 | 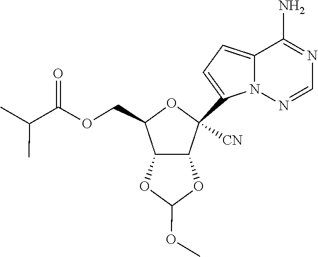 |
| Compound 81 | 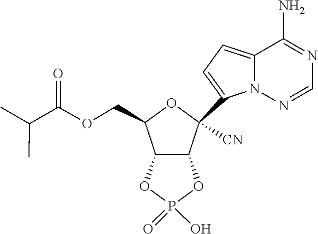 |
| Compound 82 | 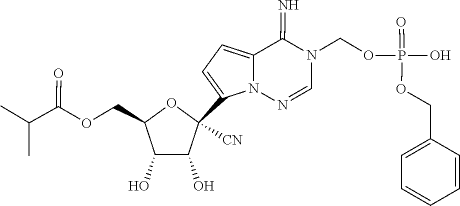 [SMILES: CC(C)C(=O)OC[C@H]1O[C@@](C#N)(c2ccc3c(=N)n(COP(=O)(O)OCc4ccccc4)cnn23)[C@H](O)[C@@H]1O] |
| Compound 83 | 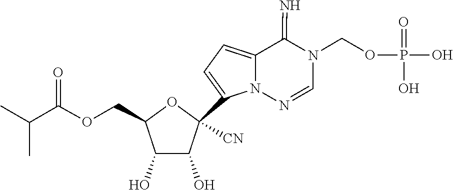 [SMILES: CC(C)C(=O)OC[C@H]1O[C@@](C#N)(c2ccc3c(=N)n(COP(=O)(O)O)cnn23)[C@H](O)[C@@H]1O] |
| Compound 84 | 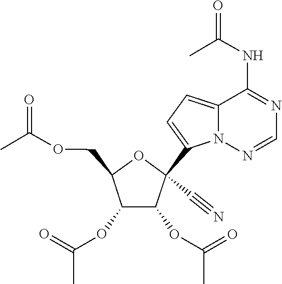 [SMILES: CC(=O)Nc1ncnn2c([C@]3(C#N)O[C@H](COC(C)=O)[C@@H](OC(C)=O)[C@H]3OC(C)=O)ccc12] |
| Compound 85 | 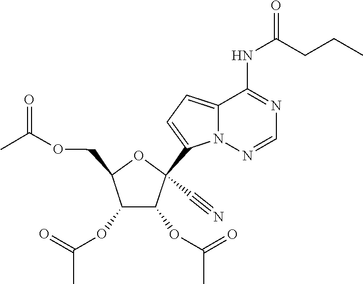 [SMILES: CCCC(=O)Nc1ncnn2c([C@]3(C#N)O[C@H](COC(C)=O)[C@@H](OC(C)=O)[C@H]3OC(C)=O)ccc12] |
| Compound 86 | 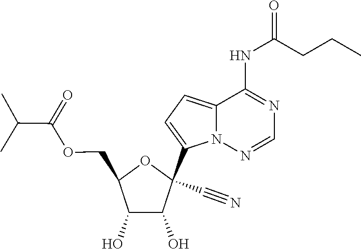 [SMILES: CCCC(=O)Nc1ncnn2c([C@]3(C#N)O[C@H](COC(=O)C(C)C)[C@@H](O)[C@H]3O)ccc12] |
| Compound 87 | 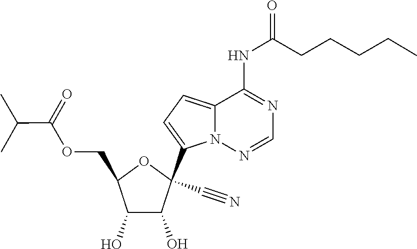 [SMILES: CCCCCC(=O)Nc1ncnn2c([C@]3(C#N)O[C@H](COC(=O)C(C)C)[C@@H](O)[C@H]3O)ccc12] |
| Compound 88 | 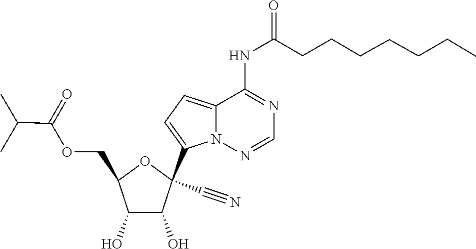 [SMILES: CCCCCCCC(=O)Nc1ncnn2c([C@]3(C#N)O[C@H](COC(=O)C(C)C)[C@@H](O)[C@H]3O)ccc12] |
| Compound 89 | 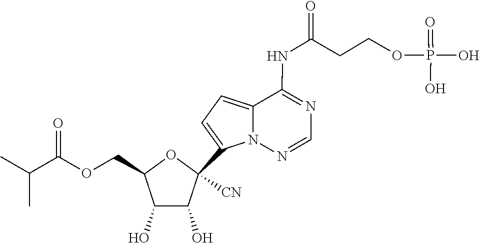 [SMILES: CC(C)C(=O)OC[C@H]1O[C@@](C#N)(c2ccc3c(NC(=O)CCOP(=O)(O)O)ncnn23)[C@H](O)[C@@H]1O] |
| Compound 90 | 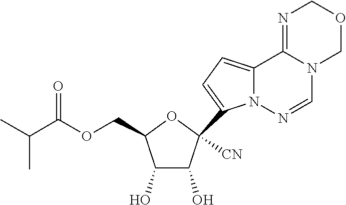 [SMILES: CC(C)C(=O)OC[C@H]1O[C@@](C#N)(c2ccc3n2N=CN2COCN=C32)[C@H](O)[C@@H]1O] |
| Compound 91 | 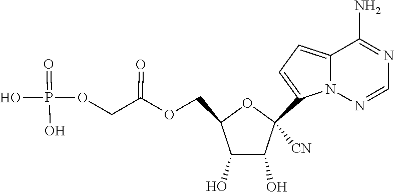 [SMILES: N#C[C@@]1(c2ccc3c(N)ncnn23)O[C@H](COC(=O)COP(=O)(O)O)[C@@H](O)[C@H]1O] |

In some embodiments, the deuterated compound of Formula A, the prodrug of the compound of Formula A, or the prodrug of the deuterated compound of Formula A is a compound of Table 3, or a pharmaceutically acceptable salt thereof.

TABLE 3

| | |
|---|---|
| Compound 92 | 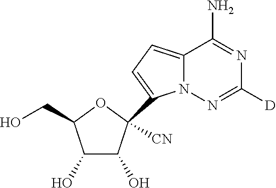 [SMILES: [2H]c1nc(N)c2ccc([C@]3(C#N)O[C@H](CO)[C@@H](O)[C@H]3O)n2n1] |
| Compound 93 | 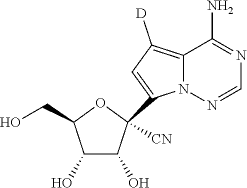 [SMILES: [2H]c1cc([C@]2(C#N)O[C@H](CO)[C@@H](O)[C@H]2O)n2ncnc(N)c12] |
| Compound 94 | 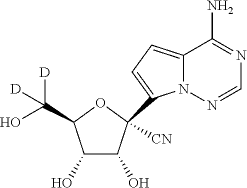 [SMILES: [2H]C([2H])(O)[C@H]1O[C@@](C#N)(c2ccc3c(N)ncnn23)[C@H](O)[C@@H]1O] |
| Compound 95 | 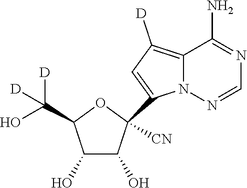 [SMILES: [2H]c1cc([C@]2(C#N)O[C@H](C([2H])([2H])O)[C@@H](O)[C@H]2O)n2ncnc(N)c12] |
| Compound 96 | 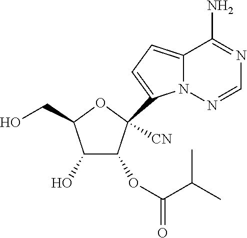 [SMILES: CC(C)C(=O)O[C@@H]1[C@H](O)[C@@H](CO)O[C@@]1(C#N)c1ccc2c(N)ncnn12] |
| Compound 97 | 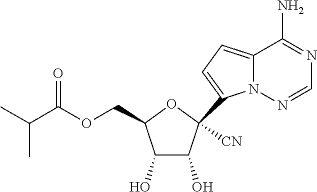 [SMILES: CC(C)C(=O)OC[C@H]1O[C@@](C#N)(c2ccc3c(N)ncnn23)[C@H](O)[C@@H]1O] |
| Compound 98 | 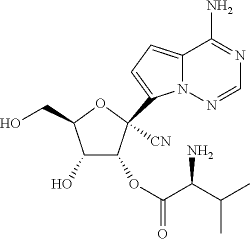 [SMILES: CC(C)[C@H](N)C(=O)O[C@@H]1[C@H](O)[C@@H](CO)O[C@@]1(C#N)c1ccc2c(N)ncnn12] |
| Compound 99 | 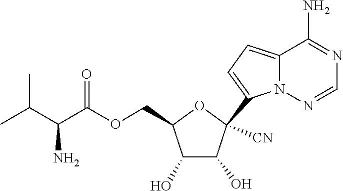 [SMILES: CC(C)[C@H](N)C(=O)OC[C@H]1O[C@@](C#N)(c2ccc3c(N)ncnn23)[C@H](O)[C@@H]1O] |
| Compound 100 | 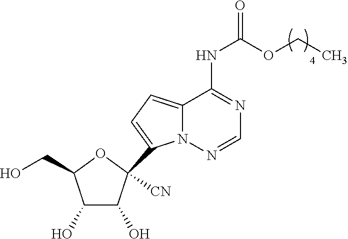 |
| Compound 101 | 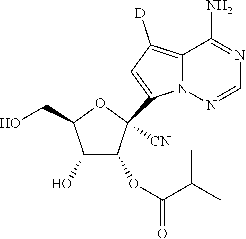 [SMILES: [2H]c1cc([C@]2(C#N)O[C@H](CO)[C@@H](O)[C@H]2OC(=O)C(C)C)n2ncnc(N)c12] |
| Compound 102 | 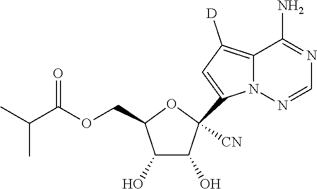 [SMILES: [2H]c1cc([C@]2(C#N)O[C@H](COC(=O)C(C)C)[C@@H](O)[C@H]2O)n2ncnc(N)c12] |
| Compound 103 | 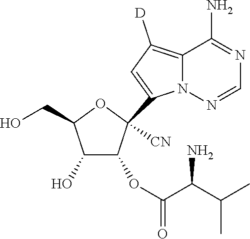 [SMILES: [2H]c1cc([C@]2(C#N)O[C@H](CO)[C@@H](O)[C@H]2OC(=O)[C@@H](N)C(C)C)n2ncnc(N)c12] |
| Compound 104 | 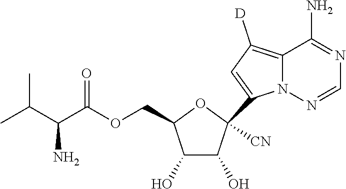 [SMILES: [2H]c1cc([C@]2(C#N)O[C@H](COC(=O)[C@@H](N)C(C)C)[C@@H](O)[C@H]2O)n2ncnc(N)c12] |
| Compound 105 | 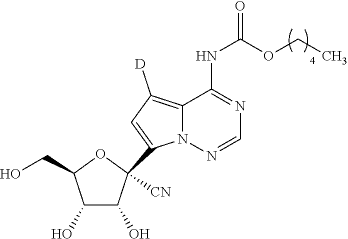 |
| Compound 106 | 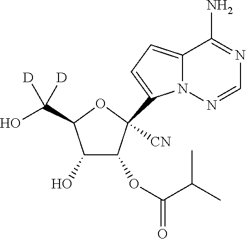 [SMILES: [2H]C([2H])(O)[C@H]1O[C@@](C#N)(c2ccc3c(N)ncnn23)[C@H](OC(=O)C(C)C)[C@@H]1O] |
| Compound 107 | 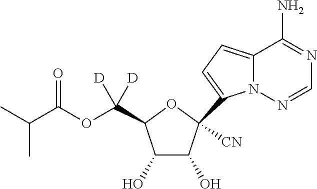 [SMILES: [2H]C([2H])(OC(=O)C(C)C)[C@H]1O[C@@](C#N)(c2ccc3c(N)ncnn23)[C@H](O)[C@@H]1O] |
| Compound 108 | 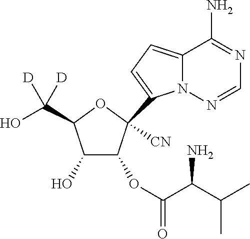 [SMILES: [2H]C([2H])(O)[C@H]1O[C@@](C#N)(c2ccc3c(N)ncnn23)[C@H](OC(=O)[C@@H](N)C(C)C)[C@@H]1O] |
| Compound 109 | 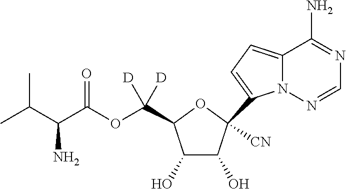 [SMILES: [2H]C([2H])(OC(=O)[C@@H](N)C(C)C)[C@H]1O[C@@](C#N)(c2ccc3c(N)ncnn23)[C@H](O)[C@@H]1O] |
| Compound 110 | 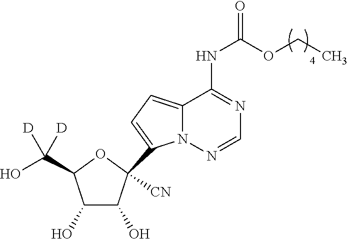 |
| Compound 111 | 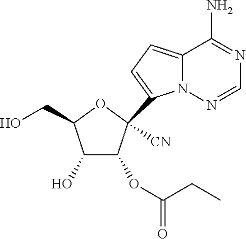 [SMILES: CCC(=O)O[C@@H]1[C@H](O)[C@@H](CO)O[C@@]1(C#N)c1ccc2c(N)ncnn12] |
| Compound 112 | 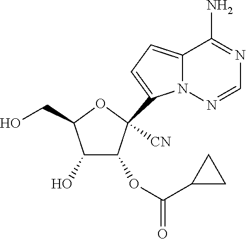 [SMILES: N#C[C@@]1(c2ccc3c(N)ncnn23)O[C@H](CO)[C@@H](O)[C@H]1OC(=O)C1CC1] |
| Compound 113 | 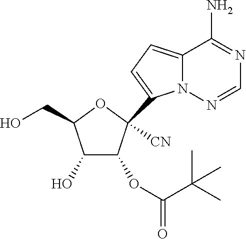 [SMILES: CC(C)(C)C(=O)O[C@@H]1[C@H](O)[C@@H](CO)O[C@@]1(C#N)c1ccc2c(N)ncnn12] |
| Compound 114 | 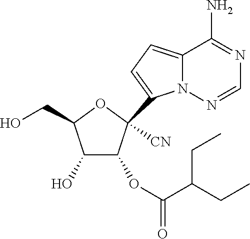 [SMILES: CCC(CC)C(=O)O[C@@H]1[C@H](O)[C@@H](CO)O[C@@]1(C#N)c1ccc2c(N)ncnn12] |
| Compound 115 | 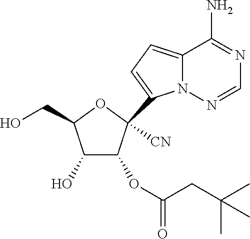 [SMILES: CC(C)(C)CC(=O)O[C@@H]1[C@H](O)[C@@H](CO)O[C@@]1(C#N)c1ccc2c(N)ncnn12] |
| Compound 116 | 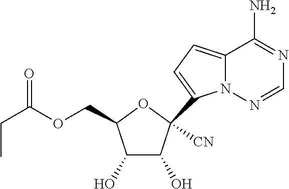 [SMILES: CCC(=O)OC[C@H]1O[C@@](C#N)(c2ccc3c(N)ncnn23)[C@H](O)[C@@H]1O] |
| Compound 117 | 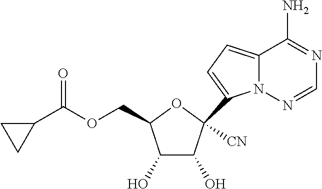 [SMILES: N#C[C@@]1(c2ccc3c(N)ncnn23)O[C@H](COC(=O)C2CC2)[C@@H](O)[C@H]1O] |
| Compound 118 | 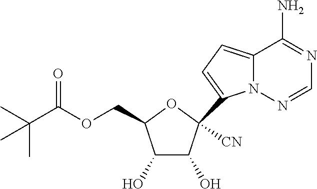 [SMILES: CC(C)(C)C(=O)OC[C@H]1O[C@@](C#N)(c2ccc3c(N)ncnn23)[C@H](O)[C@@H]1O] |
| Compound 119 | 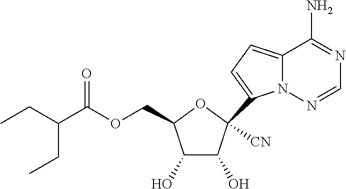 [SMILES: CCC(CC)C(=O)OC[C@H]1O[C@@](C#N)(c2ccc3c(N)ncnn23)[C@H](O)[C@@H]1O] |
| Compound 120 | 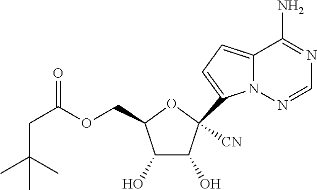 [SMILES: CC(C)(C)CC(=O)OC[C@H]1O[C@@](C#N)(c2ccc3c(N)ncnn23)[C@H](O)[C@@H]1O] |
| Compound 121 | 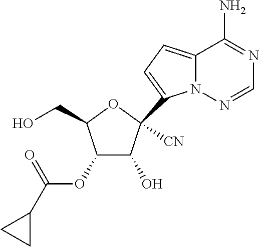 [SMILES: N#C[C@@]1(c2ccc3c(N)ncnn23)O[C@H](CO)[C@@H](OC(=O)C2CC2)[C@H]1O] |
| Compound 122 | 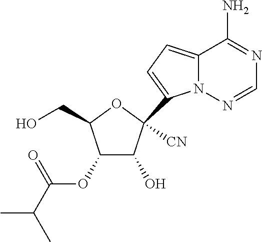 [SMILES: CC(C)C(=O)O[C@H]1[C@@H](O)[C@](C#N)(c2ccc3c(N)ncnn23)O[C@@H]1CO] |
| Compound 123 | 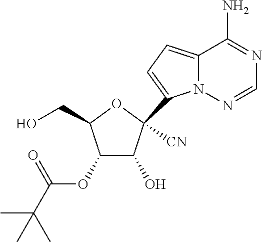 [SMILES: CC(C)(C)C(=O)O[C@H]1[C@@H](O)[C@](C#N)(c2ccc3c(N)ncnn23)O[C@@H]1CO] |
| Compound 124 | 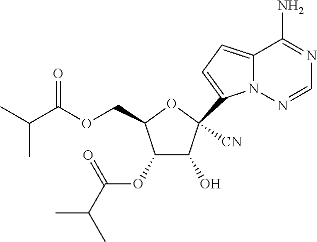 [SMILES: CC(C)C(=O)OC[C@H]1O[C@@](C#N)(c2ccc3c(N)ncnn23)[C@H](O)[C@@H]1OC(=O)C(C)C] |
| Compound 125 | 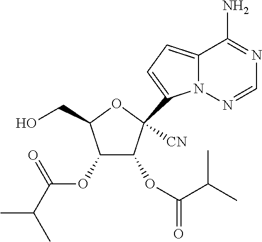 [SMILES: CC(C)C(=O)O[C@H]1[C@@H](OC(=O)C(C)C)[C@](C#N)(c2ccc3c(N)ncnn23)O[C@@H]1CO] |
| Compound 126 | 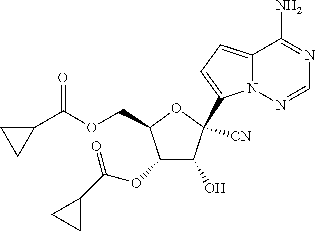 [SMILES: N#C[C@@]1(c2ccc3c(N)ncnn23)O[C@H](COC(=O)C2CC2)[C@@H](OC(=O)C2CC2)[C@H]1O] |
| Compound 127 | 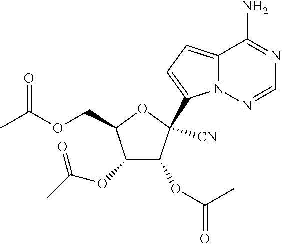 [SMILES: CC(=O)OC[C@H]1O[C@@](C#N)(c2ccc3c(N)ncnn23)[C@H](OC(C)=O)[C@@H]1OC(C)=O] |
| Compound 128 | 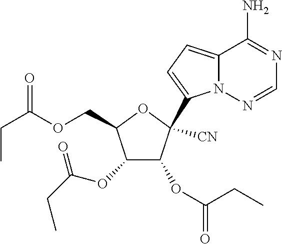 [SMILES: CCC(=O)OC[C@H]1O[C@@](C#N)(c2ccc3c(N)ncnn23)[C@H](OC(=O)CC)[C@@H]1OC(=O)CC] |
| Compound 129 | 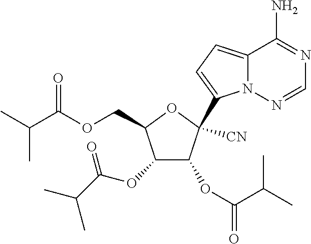 [SMILES: CC(C)C(=O)OC[C@H]1O[C@@](C#N)(c2ccc3c(N)ncnn23)[C@H](OC(=O)C(C)C)[C@@H]1OC(=O)C(C)C] |
| Compound 130 | 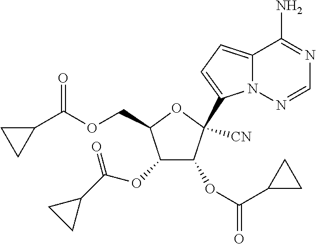 [SMILES: N#C[C@@]1(c2ccc3c(N)ncnn23)O[C@H](COC(=O)C2CC2)[C@@H](OC(=O)C2CC2)[C@H]1OC(=O)C1CC1] |
| Compound 131 | 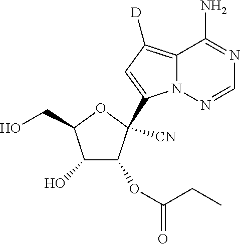 [SMILES: [2H]c1cc([C@]2(C#N)O[C@H](CO)[C@@H](O)[C@H]2OC(=O)CC)n2ncnc(N)c12] |
| Compound 132 | 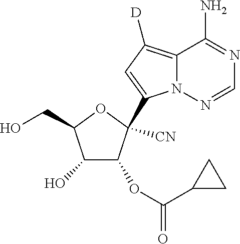 [SMILES: [2H]c1cc([C@]2(C#N)O[C@H](CO)[C@@H](O)[C@H]2OC(=O)C2CC2)n2ncnc(N)c12] |
| Compound 133 | 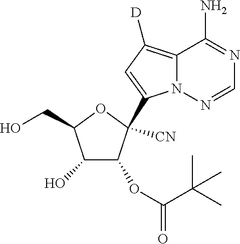 [SMILES: [2H]c1cc([C@]2(C#N)O[C@H](CO)[C@@H](O)[C@H]2OC(=O)C(C)(C)C)n2ncnc(N)c12] |
| Compound 134 | 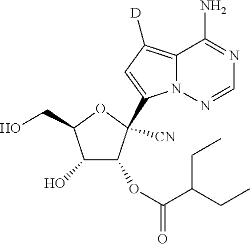 [SMILES: [2H]c1cc([C@]2(C#N)O[C@H](CO)[C@@H](O)[C@H]2OC(=O)C(CC)CC)n2ncnc(N)c12] |
| Compound 135 | 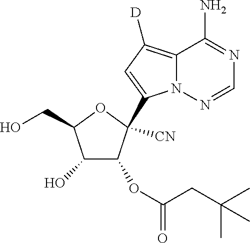 [SMILES: [2H]c1cc([C@]2(C#N)O[C@H](CO)[C@@H](O)[C@H]2OC(=O)CC(C)(C)C)n2ncnc(N)c12] |
| Compound 136 | 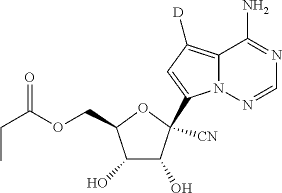 [SMILES: [2H]c1cc([C@]2(C#N)O[C@H](COC(=O)CC)[C@@H](O)[C@H]2O)n2ncnc(N)c12] |
| Compound 137 | 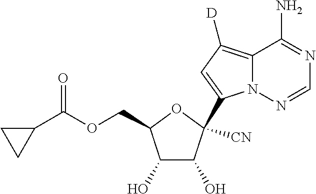 [SMILES: [2H]c1cc([C@]2(C#N)O[C@H](COC(=O)C3CC3)[C@@H](O)[C@H]2O)n2ncnc(N)c12] |
| Compound 138 | 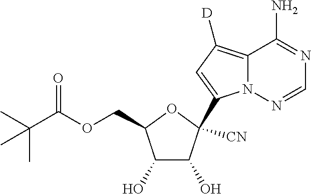 [SMILES: [2H]c1cc([C@]2(C#N)O[C@H](COC(=O)C(C)(C)C)[C@@H](O)[C@H]2O)n2ncnc(N)c12] |
| Compound 139 | 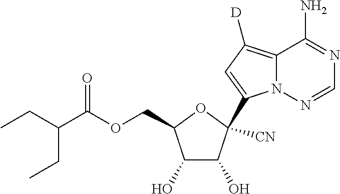 [SMILES: [2H]c1cc([C@]2(C#N)O[C@H](COC(=O)C(CC)CC)[C@@H](O)[C@H]2O)n2ncnc(N)c12] |
| Compound 140 | 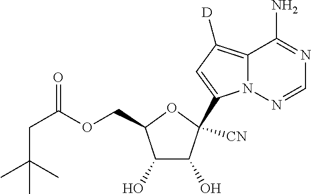 [SMILES: [2H]c1cc([C@]2(C#N)O[C@H](COC(=O)CC(C)(C)C)[C@@H](O)[C@H]2O)n2ncnc(N)c12] |
| Compound 141 | 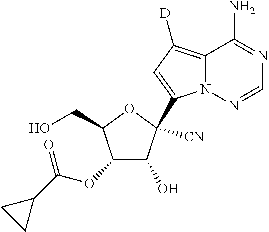 [SMILES: [2H]c1cc([C@]2(C#N)O[C@H](CO)[C@@H](OC(=O)C3CC3)[C@H]2O)n2ncnc(N)c12] |
| Compound 142 | 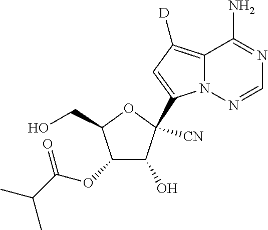 [SMILES: [2H]c1cc([C@]2(C#N)O[C@H](CO)[C@@H](OC(=O)C(C)C)[C@H]2O)n2ncnc(N)c12] |
| Compound 143 | 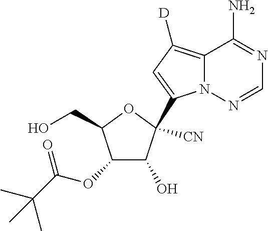 [SMILES: [2H]c1cc([C@]2(C#N)O[C@H](CO)[C@@H](OC(=O)C(C)(C)C)[C@H]2O)n2ncnc(N)c12] |
| Compound 144 | 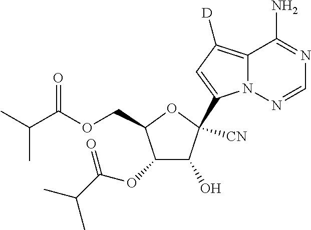 [SMILES: [2H]c1cc([C@]2(C#N)O[C@H](COC(=O)C(C)C)[C@@H](OC(=O)C(C)C)[C@H]2O)n2ncnc(N)c12] |
| Compound 145 | 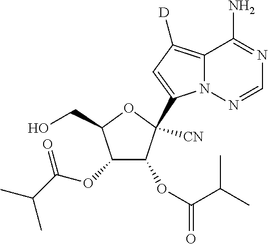 [SMILES: [2H]c1cc([C@]2(C#N)O[C@H](CO)[C@@H](OC(=O)C(C)C)[C@H]2OC(=O)C(C)C)n2ncnc(N)c12] |
| Compound 146 | 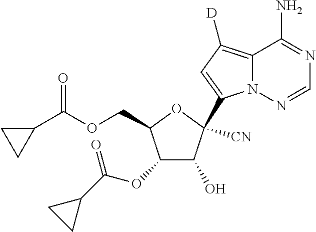 [SMILES: [2H]c1cc([C@]2(C#N)O[C@H](COC(=O)C3CC3)[C@@H](OC(=O)C3CC3)[C@H]2O)n2ncnc(N)c12] |
| Compound 147 | 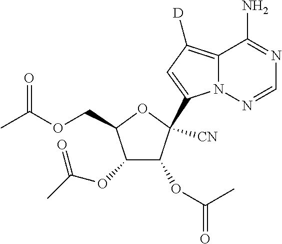 [SMILES: [2H]c1cc([C@]2(C#N)O[C@H](COC(C)=O)[C@@H](OC(C)=O)[C@H]2OC(C)=O)n2ncnc(N)c12] |
| Compound 148 | 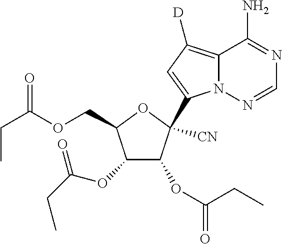 [SMILES: [2H]c1cc([C@]2(C#N)O[C@H](COC(=O)CC)[C@@H](OC(=O)CC)[C@H]2OC(=O)CC)n2ncnc(N)c12] |
| Compound 149 | 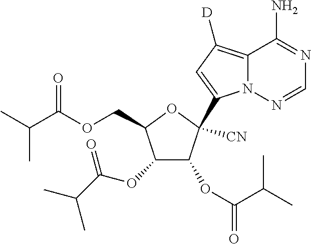 [SMILES: [2H]c1cc([C@]2(C#N)O[C@H](COC(=O)C(C)C)[C@@H](OC(=O)C(C)C)[C@H]2OC(=O)C(C)C)n2ncnc(N)c12] |
| Compound 150 | 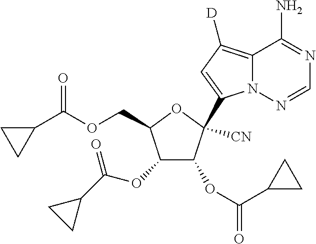 [SMILES: [2H]c1cc([C@]2(C#N)O[C@H](COC(=O)C3CC3)[C@@H](OC(=O)C3CC3)[C@H]2OC(=O)C2CC2)n2ncnc(N)c12] |
| Compound 151 | 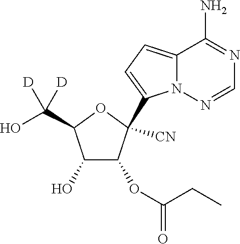 [SMILES: [2H]C([2H])(O)[C@H]1O[C@@](C#N)(c2ccc3c(N)ncnn23)[C@H](OC(=O)CC)[C@@H]1O] |
| Compound 152 | 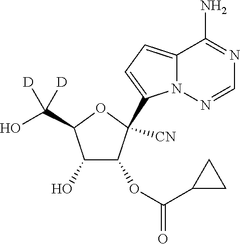 [SMILES: [2H]C([2H])(O)[C@H]1O[C@@](C#N)(c2ccc3c(N)ncnn23)[C@H](OC(=O)C2CC2)[C@@H]1O] |
| Compound 153 | 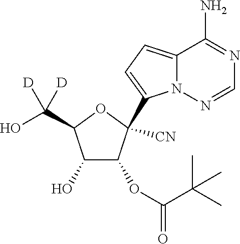 [SMILES: [2H]C([2H])(O)[C@H]1O[C@@](C#N)(c2ccc3c(N)ncnn23)[C@H](OC(=O)C(C)(C)C)[C@@H]1O] |
| Compound 154 | 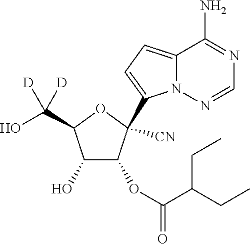 [SMILES: [2H]C([2H])(O)[C@H]1O[C@@](C#N)(c2ccc3c(N)ncnn23)[C@H](OC(=O)C(CC)CC)[C@@H]1O] |
| Compound 155 | 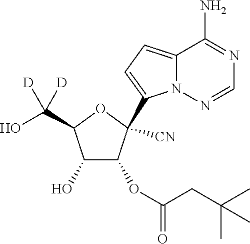 [SMILES: [2H]C([2H])(O)[C@H]1O[C@@](C#N)(c2ccc3c(N)ncnn23)[C@H](OC(=O)CC(C)(C)C)[C@@H]1O] |
| Compound 156 | 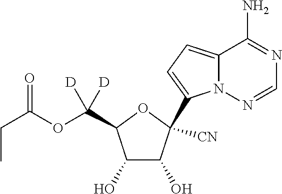 [SMILES: [2H]C([2H])(OC(=O)CC)[C@H]1O[C@@](C#N)(c2ccc3c(N)ncnn23)[C@H](O)[C@@H]1O] |
| Compound 157 | 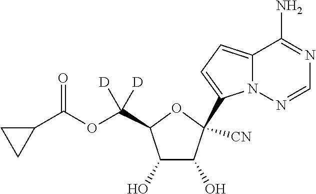 [SMILES: [2H]C([2H])(OC(=O)C1CC1)[C@H]1O[C@@](C#N)(c2ccc3c(N)ncnn23)[C@H](O)[C@@H]1O] |
| Compound 158 | 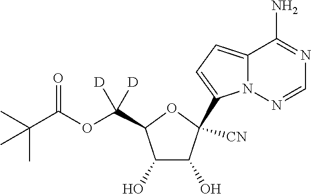 [SMILES: [2H]C([2H])(OC(=O)C(C)(C)C)[C@H]1O[C@@](C#N)(c2ccc3c(N)ncnn23)[C@H](O)[C@@H]1O] |
| Compound 159 | 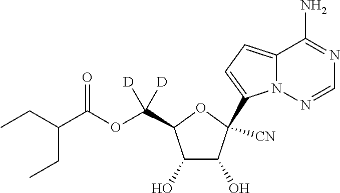 [SMILES: [2H]C([2H])(OC(=O)C(CC)CC)[C@H]1O[C@@](C#N)(c2ccc3c(N)ncnn23)[C@H](O)[C@@H]1O] |
| Compound 160 | 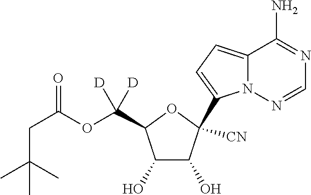 [SMILES: [2H]C([2H])(OC(=O)CC(C)(C)C)[C@H]1O[C@@](C#N)(c2ccc3c(N)ncnn23)[C@H](O)[C@@H]1O] |
| Compound 161 | 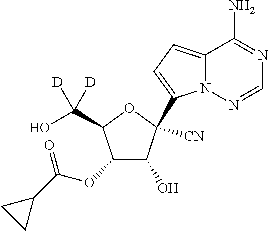 [SMILES: [2H]C([2H])(O)[C@H]1O[C@@](C#N)(c2ccc3c(N)ncnn23)[C@H](O)[C@@H]1OC(=O)C1CC1] |
| Compound 162 | 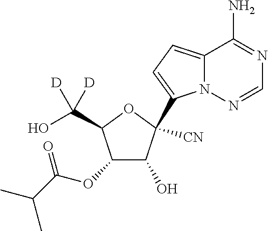 [SMILES: [2H]C([2H])(O)[C@H]1O[C@@](C#N)(c2ccc3c(N)ncnn23)[C@H](O)[C@@H]1OC(=O)C(C)C] |
| Compound 163 | 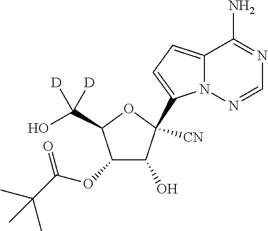 [SMILES: [2H]C([2H])(O)[C@H]1O[C@@](C#N)(c2ccc3c(N)ncnn23)[C@H](O)[C@@H]1OC(=O)C(C)(C)C] |
| Compound 164 | 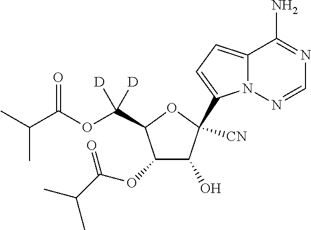 [SMILES: [2H]C([2H])(OC(=O)C(C)C)[C@H]1O[C@@](C#N)(c2ccc3c(N)ncnn23)[C@H](O)[C@@H]1OC(=O)C(C)C] |
| Compound 165 | 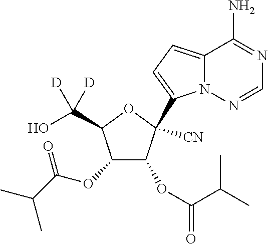 [SMILES: [2H]C([2H])(O)[C@H]1O[C@@](C#N)(c2ccc3c(N)ncnn23)[C@H](OC(=O)C(C)C)[C@@H]1OC(=O)C(C)C] |
| Compound 166 | 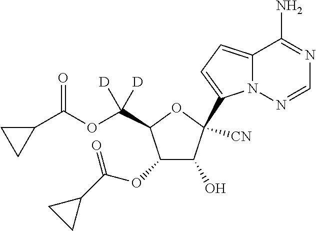 [SMILES: [2H]C([2H])(OC(=O)C1CC1)[C@H]1O[C@@](C#N)(c2ccc3c(N)ncnn23)[C@H](O)[C@@H]1OC(=O)C1CC1] |
| Compound 167 | 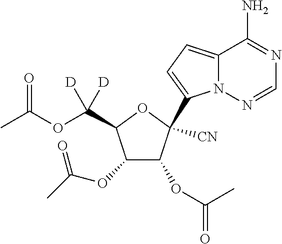 [SMILES: [2H]C([2H])(OC(C)=O)[C@H]1O[C@@](C#N)(c2ccc3c(N)ncnn23)[C@H](OC(C)=O)[C@@H]1OC(C)=O] |
| Compound 168 | 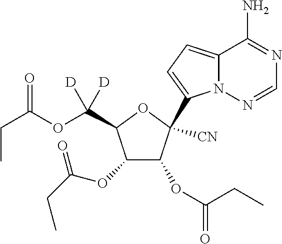 [SMILES: [2H]C([2H])(OC(=O)CC)[C@H]1O[C@@](C#N)(c2ccc3c(N)ncnn23)[C@H](OC(=O)CC)[C@@H]1OC(=O)CC] |
| Compound 169 | 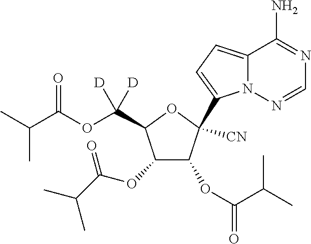 [SMILES: [2H]C([2H])(OC(=O)C(C)C)[C@H]1O[C@@](C#N)(c2ccc3c(N)ncnn23)[C@H](OC(=O)C(C)C)[C@@H]1OC(=O)C(C)C] |
| Compound 170 | 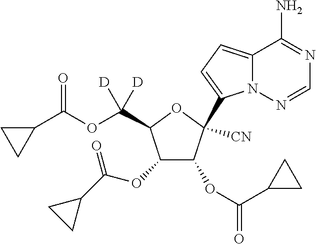 [SMILES: [2H]C([2H])(OC(=O)C1CC1)[C@H]1O[C@@](C#N)(c2ccc3c(N)ncnn23)[C@H](OC(=O)C2CC2)[C@@H]1OC(=O)C1CC1] |
| Compound 171 | 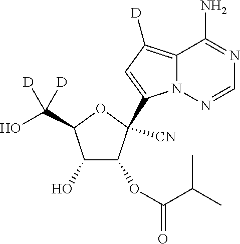 [SMILES: [2H]c1cc([C@]2(C#N)O[C@H](C([2H])([2H])O)[C@@H](O)[C@H]2OC(=O)C(C)C)n2ncnc(N)c12] |
| Compound 172 | 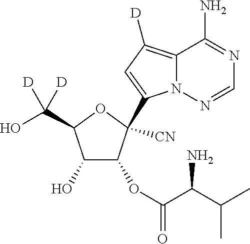 [SMILES: [2H]c1cc([C@]2(C#N)O[C@H](C([2H])([2H])O)[C@@H](O)[C@H]2OC(=O)[C@@H](N)C(C)C)n2ncnc(N)c12] |
| Compound 173 | 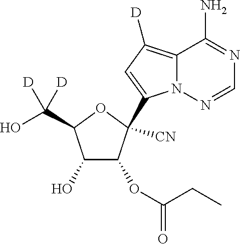 [SMILES: [2H]c1cc([C@]2(C#N)O[C@H](C([2H])([2H])O)[C@@H](O)[C@H]2OC(=O)CC)n2ncnc(N)c12] |
| Compound 174 | 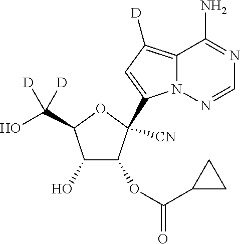 [SMILES: [2H]c1cc([C@]2(C#N)O[C@H](C([2H])([2H])O)[C@@H](O)[C@H]2OC(=O)C2CC2)n2ncnc(N)c12] |
| Compound 175 | 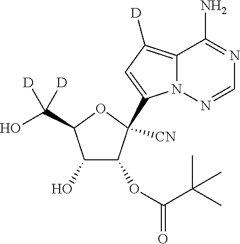 [SMILES: [2H]c1cc([C@]2(C#N)O[C@H](C([2H])([2H])O)[C@@H](O)[C@H]2OC(=O)C(C)(C)C)n2ncnc(N)c12] |
| Compound 176 | 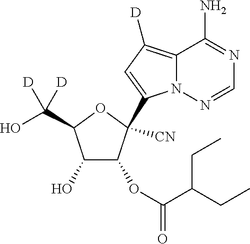 [SMILES: [2H]c1cc([C@]2(C#N)O[C@H](C([2H])([2H])O)[C@@H](O)[C@H]2OC(=O)C(CC)CC)n2ncnc(N)c12] |
| Compound 177 | 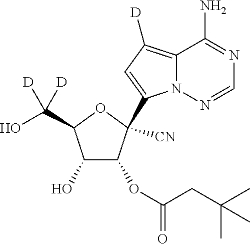 [SMILES: [2H]c1cc([C@]2(C#N)O[C@H](C([2H])([2H])O)[C@@H](O)[C@H]2OC(=O)CC(C)(C)C)n2ncnc(N)c12] |
| Compound 178 | 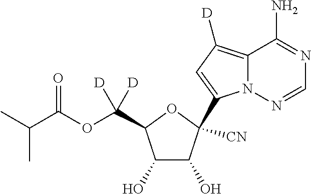 [SMILES: [2H]c1cc([C@]2(C#N)O[C@H](C([2H])([2H])OC(=O)C(C)C)[C@@H](O)[C@H]2O)n2ncnc(N)c12] |
| Compound 179 | 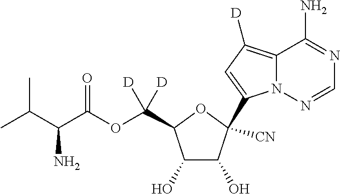 [SMILES: [2H]c1cc([C@]2(C#N)O[C@H](C([2H])([2H])OC(=O)[C@@H](N)C(C)C)[C@@H](O)[C@H]2O)n2ncnc(N)c12] |
| Compound 180 | 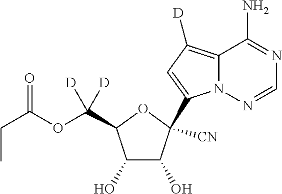 [SMILES: [2H]c1cc([C@]2(C#N)O[C@H](C([2H])([2H])OC(=O)CC)[C@@H](O)[C@H]2O)n2ncnc(N)c12] |
| Compound 181 | 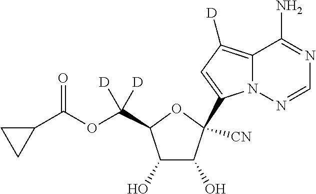 [SMILES: [2H]c1cc([C@]2(C#N)O[C@H](C([2H])([2H])OC(=O)C3CC3)[C@@H](O)[C@H]2O)n2ncnc(N)c12] |
| Compound 182 | 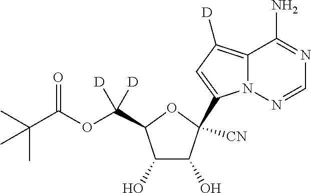 [SMILES: [2H]c1cc([C@]2(C#N)O[C@H](C([2H])([2H])OC(=O)C(C)(C)C)[C@@H](O)[C@H]2O)n2ncnc(N)c12] |
| Compound 183 | 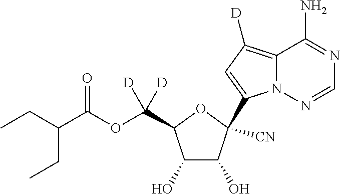 [SMILES: [2H]c1cc([C@]2(C#N)O[C@H](C([2H])([2H])OC(=O)C(CC)CC)[C@@H](O)[C@H]2O)n2ncnc(N)c12] |
| Compound 184 | 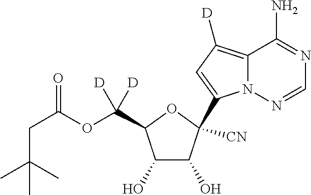 [SMILES: [2H]c1cc([C@]2(C#N)O[C@H](C([2H])([2H])OC(=O)CC(C)(C)C)[C@@H](O)[C@H]2O)n2ncnc(N)c12] |
| Compound 185 | 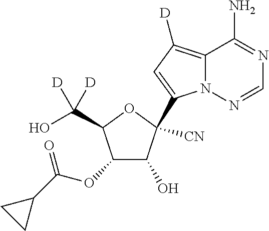 [SMILES: [2H]c1cc([C@]2(C#N)O[C@H](C([2H])([2H])O)[C@@H](OC(=O)C3CC3)[C@H]2O)n2ncnc(N)c12] |
| Compound 186 | 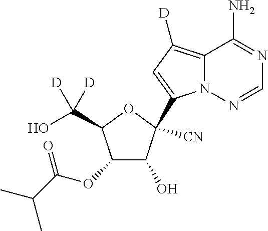 [SMILES: [2H]c1cc([C@]2(C#N)O[C@H](C([2H])([2H])O)[C@@H](OC(=O)C(C)C)[C@H]2O)n2ncnc(N)c12] |
| Compound 187 | 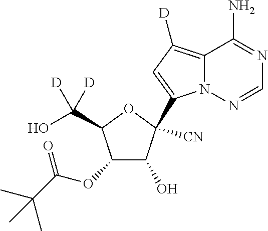 [SMILES: [2H]c1cc([C@]2(C#N)O[C@H](C([2H])([2H])O)[C@@H](OC(=O)C(C)(C)C)[C@H]2O)n2ncnc(N)c12] |
| Compound 188 | 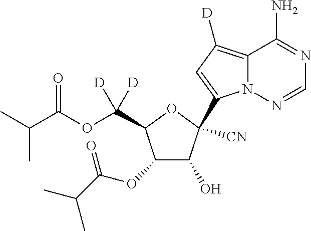 [SMILES: [2H]c1cc([C@]2(C#N)O[C@H](C([2H])([2H])OC(=O)C(C)C)[C@@H](OC(=O)C(C)C)[C@H]2O)n2ncnc(N)c12] |
| Compound 189 | 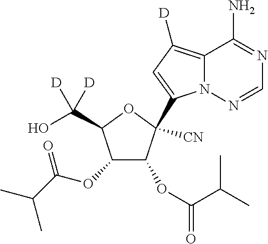 [SMILES: [2H]c1cc([C@]2(C#N)O[C@H](C([2H])([2H])O)[C@@H](OC(=O)C(C)C)[C@H]2OC(=O)C(C)C)n2ncnc(N)c12] |
| Compound 190 | 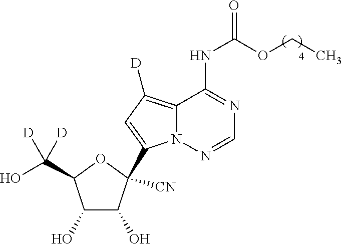 |
| Compound 191 | 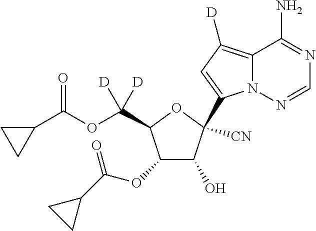 [SMILES: [2H]c1cc([C@]2(C#N)O[C@H](C([2H])([2H])OC(=O)C3CC3)[C@@H](OC(=O)C3CC3)[C@H]2O)n2ncnc(N)c12] |
| Compound 192 | 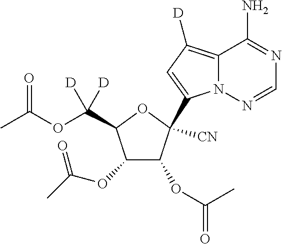 [SMILES: [2H]c1cc([C@]2(C#N)O[C@H](C([2H])([2H])OC(C)=O)[C@@H](OC(C)=O)[C@H]2OC(C)=O)n2ncnc(N)c12] |
| Compound 193 | 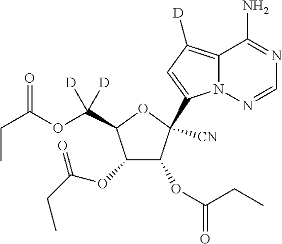 [SMILES: [2H]c1cc([C@]2(C#N)O[C@H](C([2H])([2H])OC(=O)CC)[C@@H](OC(=O)CC)[C@H]2OC(=O)CC)n2ncnc(N)c12] |
| Compound 194 | 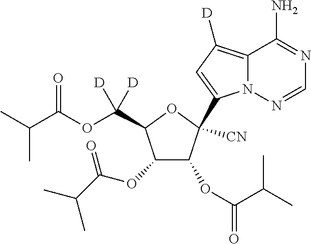 [SMILES: [2H]c1cc([C@]2(C#N)O[C@H](C([2H])([2H])OC(=O)C(C)C)[C@@H](OC(=O)C(C)C)[C@H]2OC(=O)C(C)C)n2ncnc(N)c12] |
| Compound 195 | 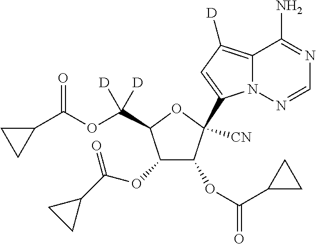 [SMILES: [2H]c1cc([C@]2(C#N)O[C@H](C([2H])([2H])OC(=O)C3CC3)[C@@H](OC(=O)C3CC3)[C@H]2OC(=O)C2CC2)n2ncnc(N)c12] |
| Compound 196 | 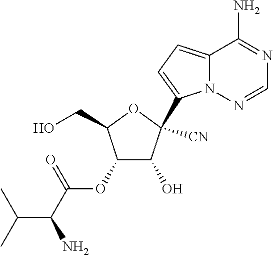 [SMILES: CC(C)[C@H](N)C(=O)O[C@H]1[C@@H](O)[C@](C#N)(c2ccc3c(N)ncnn23)O[C@@H]1CO] |
| Compound 197 | 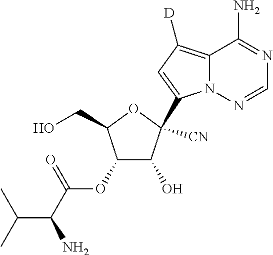 [SMILES: [2H]c1cc([C@]2(C#N)O[C@H](CO)[C@@H](OC(=O)[C@@H](N)C(C)C)[C@H]2O)n2ncnc(N)c12] |
| Compound 198 | 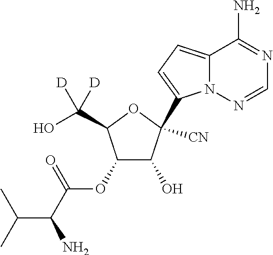 [SMILES: [2H]C([2H])(O)[C@H]1O[C@@](C#N)(c2ccc3c(N)ncnn23)[C@H](O)[C@@H]1OC(=O)[C@@H](N)C(C)C] |
| Compound 199 | 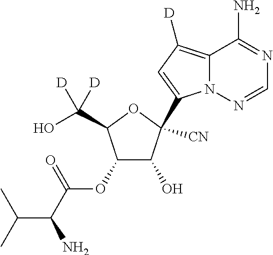 [SMILES: [2H]c1cc([C@]2(C#N)O[C@H](C([2H])([2H])O)[C@@H](OC(=O)[C@@H](N)C(C)C)[C@H]2O)n2ncnc(N)c12] |
| Compound 200 | 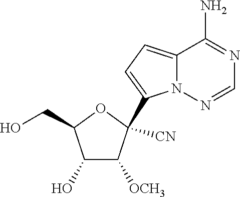 [SMILES: CO[C@@H]1[C@H](O)[C@@H](CO)O[C@@]1(C#N)c1ccc2c(N)ncnn12] |

In some embodiments, the compound of Formula I is a compound of Table 4, or a pharmaceutically acceptable salt thereof.

TABLE 4

| | |
|---|---|
| Compound 201 | 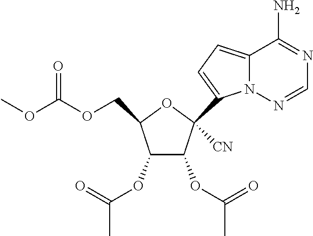 [SMILES: COC(=O)OC[C@H]1O[C@@](C#N)(c2ccc3c(N)ncnn23)[C@H](OC(C)=O)[C@@H]1OC(C)=O] |
| Compound 202 | 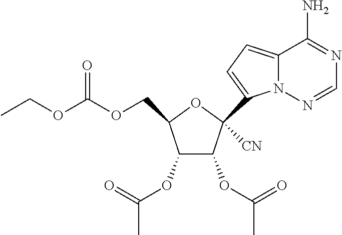 [SMILES: CCOC(=O)OC[C@H]1O[C@@](C#N)(c2ccc3c(N)ncnn23)[C@H](OC(C)=O)[C@@H]1OC(C)=O] |
| Compound 203 | 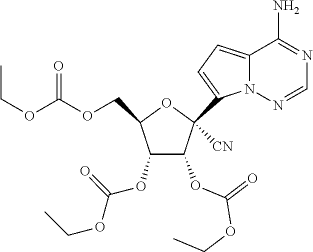 [SMILES: CCOC(=O)OC[C@H]1O[C@@](C#N)(c2ccc3c(N)ncnn23)[C@H](OC(=O)OCC)[C@@H]1OC(=O)OCC] |
| Compound 204 | 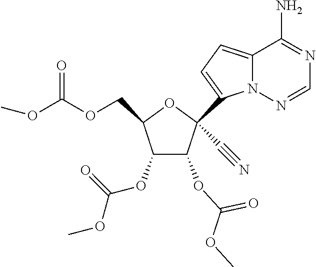 [SMILES: COC(=O)OC[C@H]1O[C@@](C#N)(c2ccc3c(N)ncnn23)[C@H](OC(=O)OC)[C@@H]1OC(=O)OC] |

In some embodiments, the compound of Formula I is

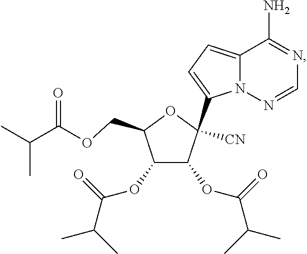 [SMILES: CC(C)C(=O)OC[C@H]1O[C@@](C#N)(c2ccc3c(N)ncnn23)[C@H](OC(=O)C(C)C)[C@@H]1OC(=O)C(C)C]

or a pharmaceutically acceptable salt thereof.

In some embodiments, the deuterated compound of Formula I is

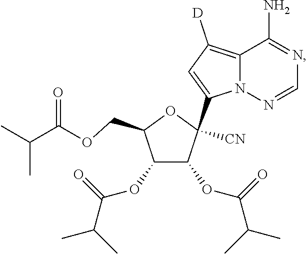 [SMILES: [2H]c1cc([C@]2(C#N)O[C@H](COC(=O)C(C)C)[C@@H](OC(=O)C(C)C)[C@H]2OC(=O)C(C)C)n2ncnc(N)c12]

or a pharmaceutically acceptable salt thereof.

In some embodiments, the compound of Formula I is

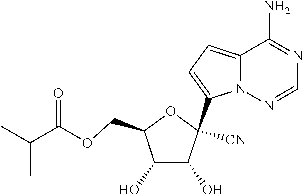 [SMILES: CC(C)C(=O)OC[C@H]1O[C@@](C#N)(c2ccc3c(N)ncnn23)[C@H](O)[C@@H]1O]

or a pharmaceutically acceptable salt thereof.

The compounds described herein may be formulated with conventional carriers and excipients. For example, tablets will contain excipients, glidants, fillers, binders and the like. Aqueous formulations are prepared in sterile form, and when intended for delivery by other than oral administration generally will be isotonic. All formulations may optionally comprise excipients such as those set forth in the “Handbook of Pharmaceutical Excipients” (1986). Pharmaceutically acceptable excipients include ascorbic acid and other antioxidants, chelating agents such as EDTA, carbohydrates such as dextran, hydroxyalkylcellulose, hydroxyalkylmethylcellulose, stearic acid and the like. In some embodiments, the formulations comprise one or more pharmaceutically acceptable excipients. The pH of the formulations ranges from about 3 to about 11, but is ordinarily about 7 to 10. In some embodiments, the pH of the formulations ranges from about 2 to about 5, but is ordinarily about 3 to 4.

While it is possible for the compounds of the disclosure (“the active ingredients”) to be administered alone it may be preferable to present them as pharmaceutical formulations. The formulations, both for veterinary and for human use, of the invention comprise at least one active ingredient, as above defined, together with one or more acceptable carriers therefor and optionally other therapeutic ingredients, particularly those additional therapeutic ingredients as discussed herein. The carrier(s) must be “acceptable” in the sense of being compatible with the other ingredients of the formulation and physiologically innocuous to the recipient thereof.

The formulations include those suitable for the foregoing administration routes. The formulations may conveniently be presented in unit dosage form and may be prepared by any appropriate method known in the art of pharmacy. Techniques and formulations generally are found in Remington's Pharmaceutical Sciences (Mack Publishing Co., Easton, PA). Such methods include the step of bringing into association the active ingredient with the carrier which constitutes one or more accessory ingredients. In general, the formulations are prepared by uniformly and intimately bringing into association the active ingredient with liquid carriers or finely divided solid carriers or both, and then, if necessary, shaping the product.

In some embodiments, the pharmaceutical formulation is for subcutaneous, intramuscular, intravenous, oral, or inhalation administration.

In some embodiments, the compound described herein e.g., the compound of Formula A, the deuterated compound of Formula A, the prodrug of the compound of Formula A, the prodrug of the deuterated compound of Formula A, the compound of Formula I, the deuterated compound of Formula I, or the pharmaceutically acceptable salt thereof, described herein have optimized/improved pharmacokinetic properties and are amenable to oral administration. For example, the compounds of Formula I have improved bioavailability and can therefore be administered by oral administration.

In some embodiments, the formulations of the present invention are suitable for oral administration may be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste.

In some embodiments, the tablet is made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

For infections of the eye or other external tissues, e.g., mouth and skin, the formulations are applied as a topical ointment or cream containing the active ingredient(s) in an amount of, for example, 0.075 to 20% w/w (including active ingredient(s) in a range between 0.1% and 20% in increments of 0.1% w/w such as 0.6% w/w, 0.7% w/w, etc.), preferably 0.2 to 15% w/w and most preferably 0.5 to 10% w/w. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include, for example, at least 30% w/w of a polyhydric alcohol, i.e., an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulphoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

Emulgents and emulsion stabilizers suitable for use in the formulation of the invention include Tween® 60, Span® 80, cetostearyl alcohol, benzyl alcohol, myristyl alcohol, glyceryl mono-stearate and sodium lauryl sulfate. Further emulgents and emulsion stabilizers suitable for use in the formulation of the invention include Tween® 80.

The choice of suitable oils or fats for the formulation is based on achieving the desired cosmetic properties. The cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils are used.

Pharmaceutical formulations according to the present invention comprise a compound according to the invention together with one or more pharmaceutically acceptable carriers or excipients and optionally other therapeutic agents. Pharmaceutical formulations containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

Aqueous suspensions of the invention contain the active materials in admixture with excipients suitable for the manufacture of aqueous suspensions. Such excipients include a suspending agent, such as sodium carboxymethylcellulose, methylcellulose, hydroxypropyl methylcelluose, sodium alginate, polyvinylpyrrolidone, gum tragacanth and gum acacia, and dispersing or wetting agents such as a naturally-occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin. Further non-limiting examples of suspending agents include Cyclodextrin. In some examples, the suspending agent is Sulfobutyl ether beta-cyclodextrin (SEB-beta-CD), for example Captisol®.

Oil suspensions may be formulated by suspending the active ingredient in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Dispersible powders and granules of the invention suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the invention may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally-occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

The pharmaceutical compositions of the invention may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose, any bland fixed oil may be employed including synthetic mono- or diglycerides. In addition, fatty acids such as oleic acid may likewise be used in the preparation of injectables. Among the acceptable vehicles and solvents that may be employed are water, Ringer's solution isotonic sodium chloride solution, and hypertonic sodium chloride solution.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 mg of the active ingredient per milliliter of solution in order that infusion of a suitable volume at a rate of about 30 mL/hr can occur.

Formulations suitable for topical administration to the eye also include eye drops wherein the active ingredient is dissolved or suspended in a suitable carrier, especially an aqueous solvent for the active ingredient. The active ingredient is preferably present in such formulations in a concentration of 0.5 to 20%, advantageously 0.5 to 10%, and particularly about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

In some embodiments, the compounds described herein are administered by inhalation. In some embodiments, formulations suitable for intrapulmonary or nasal administration have a particle size for example in the range of 0.1 to 500 microns, such as 0.5, 1, 30, 35 etc., which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents. In some embodiments, the compounds used herein are formulated and dosed as dry powder. In some embodiments, the compounds used herein are formulated and dosed as a nebulized formulation. In some embodiments, the compounds used herein are formulated for delivery by a face mask. In some embodiments, the compounds used herein are formulated for delivery by a face tent.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations are presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

It should be understood that in addition to the ingredients particularly mentioned above the formulations of this invention may include other agents conventional in the art having regard to the type of formulation in question, for example those suitable for oral administration may include flavoring agents.

The invention further provides veterinary compositions comprising at least one active ingredient as above defined together with a veterinary carrier therefor.

Veterinary carriers are materials useful for the purpose of administering the composition and may be solid, liquid or gaseous materials which are otherwise inert or acceptable in the veterinary art and are compatible with the active ingredient. These veterinary compositions may be administered orally, parenterally or by any other desired route.

Compounds described herein are used to provide controlled release pharmaceutical formulations containing as active ingredient one or more compounds described herein (“controlled release formulations”) in which the release of the active ingredient are controlled and regulated to allow less frequency dosing or to improve the pharmacokinetic or toxicity profile of a given active ingredient.

Also provided herein are kits that include a compound described herein. In some embodiments the kits described herein may comprise a label and/or instructions for use of the compound in the treatment of a disease or condition in a non-pregnant patient in need thereof. In some embodiments, the disease or condition is viral infection.

In some embodiments, the kit may also comprise one or more additional therapeutic agents and/or instructions for use of additional therapeutic agents in combination with the compound of Formula A, the deuterated compound of Formula A, the prodrug of the compound of Formula A, the prodrug of the deuterated compound of Formula A, the compound of Formula I, the deuterated compound of Formula I, or the pharmaceutically acceptable salt thereof in the treatment of the disease or condition in a subject (e.g., human) in need thereof.

In some embodiments, the kits provided herein comprises individual dose units of the compound of Formula A, the deuterated compound of Formula A, the prodrug of the compound of Formula A, the prodrug of the deuterated compound of Formula A, the compound of Formula I, the deuterated compound of Formula I, or the pharmaceutically acceptable salt thereof. Examples of individual dosage units may include pills, tablets, capsules, prefilled syringes or syringe cartridges, IV bags, inhalers, nebulizers etc., each comprising a therapeutically effective amount of the compound of Formula A, the deuterated compound of Formula A, the prodrug of the compound of Formula A, the prodrug of the deuterated compound of Formula A, the compound of Formula I, the deuterated compound of Formula I, or the pharmaceutically acceptable salt thereof. In some embodiments, the kit may contain a single dosage unit and in others, multiple dosage units are present, such as the number of dosage units required for a specified regimen or period.

One or more compounds described herein are administered by any route appropriate to the condition to be treated. Suitable routes include oral, rectal, inhalation, pulmonary, topical (including buccal and sublingual), vaginal and parenteral (including subcutaneous, intramuscular, intravenous, intradermal, intrathecal and epidural), and the like. In some embodiments, the compounds described herein are administered by inhalation or intravenously. In some embodiments, the compounds described herein are administered orally. It will be appreciated that the preferred route may vary with for example the condition of the recipient.

In the methods described herein for the treatment of a viral infection, the compounds described herein can be administered at any time to a human who may come into contact with the virus or is already suffering from the viral infection. In some embodiments, the compounds described herein can be administered prophylactically to humans coming into contact with humans suffering from the viral infection or at risk of coming into contact with humans suffering from the viral infection, e.g., healthcare providers. In some embodiments, administration of the compounds described herein can be to humans testing positive for the viral infection but not yet showing symptoms of the viral infection. In some embodiments, administration of the compounds described herein can be to humans upon commencement of symptoms of the viral infection.

In some embodiments, the methods described herein comprise event driven administration of the compound described herein, e.g., the compound of Formula A, the deuterated compound of Formula A, the prodrug of the compound of Formula A, the prodrug of the deuterated compound of Formula A, the compound of Formula I, the deuterated compound of Formula I, or the pharmaceutically acceptable salt thereof, to the subject.

As used herein, the terms “event driven” or “event driven administration” refer to administration of the compound described herein (e.g., the compound of Formula A, the deuterated compound of Formula A, the prodrug of the compound of Formula A, the prodrug of the deuterated compound of Formula A, the compound of Formula I, the deuterated compound of Formula I, or the pharmaceutically acceptable salt thereof), or a pharmaceutically acceptable salt thereof, (1) prior to an event (e.g., 2 hours, 1 day, 2 days, 5 day, or 7 or more days prior to the event) that would expose the individual to the virus (or that would otherwise increase the individual's risk of acquiring the viral infection); and/or (2) during an event (or more than one recurring event) that would expose the individual to the virus (or that would otherwise increase the individual's risk of acquiring the viral infection); and/or (3) after an event (or after the final event in a series of recurring events) that would expose the individual to the virus (or that would otherwise increase the individual's risk of acquiring the viral infection). In some embodiments, the event driven administration is performed pre-exposure of the subject to the virus. In some embodiments, the event driven administration is performed post-exposure of the subject to the virus. In some embodiments, the event driven administration is performed pre-exposure of the subject to the virus and post-exposure of the subject to the virus.

In certain embodiments, the methods described herein involve administration prior to and/or after an event that would expose the individual to the virus or that would otherwise increase the individual's risk of acquiring the viral infection, e.g., as pre-exposure prophylaxis (PrEP) and/or as post-exposure prophylaxis (PEP). In some embodiments, the methods described herein comprise pre-exposure prophylaxis (PrEP). In some embodiments, methods described herein comprise post-exposure prophylaxis (PEP).

In some embodiments, the compound of Formula A, the deuterated compound of Formula A, the prodrug of the compound of Formula A, the prodrug of the deuterated compound of Formula A, the compound of Formula I, the deuterated compound of Formula I, or the pharmaceutically acceptable salt thereof, is administered before exposure of the subject to the virus.

In some embodiments, the compound of Formula A, the deuterated compound of Formula A, the prodrug of the compound of Formula A, the prodrug of the deuterated compound of Formula A, the compound of Formula I, the deuterated compound of Formula I, or the pharmaceutically acceptable salt thereof, is administered before and after exposure of the subject to the virus.

In some embodiments, the compound of Formula A, the deuterated compound of Formula A, the prodrug of the compound of Formula A, the prodrug of the deuterated compound of Formula A, the compound of Formula I, the deuterated compound of Formula I, or the pharmaceutically acceptable salt thereof, is administered after exposure of the subject to the virus.

An example of event driven dosing regimen includes administration of the compound of Formula A, the deuterated compound of Formula A, the prodrug of the compound of Formula A, the prodrug of the deuterated compound of Formula A, the compound of Formula I, the deuterated compound of Formula I, or the pharmaceutically acceptable salt thereof, within 24 to 2 hours prior to the virus, followed by administration of the compound of Formula A, the deuterated compound of Formula A, the prodrug of the compound of Formula A, the prodrug of the deuterated compound of Formula A, the compound of Formula I, the deuterated compound of Formula I, or the pharmaceutically acceptable salt thereof, every 24 hours during the period of exposure, followed by a further administration of the compound of Formula A, the deuterated compound of Formula A, the prodrug of the compound of Formula A, the prodrug of the deuterated compound of Formula A, the compound of Formula I, the deuterated compound of Formula I, or the pharmaceutically acceptable salt thereof, after the last exposure, and one last administration of the compound of Formula A, the deuterated compound of Formula A, the prodrug of the compound of Formula A, the prodrug of the deuterated compound of Formula A, the compound of Formula I, the deuterated compound of Formula I, or the pharmaceutically acceptable salt thereof, 24 hours later.

A further example of an event driven dosing regimen includes administration of the compound of Formula A, the deuterated compound of Formula A, the prodrug of the compound of Formula A, the prodrug of the deuterated compound of Formula A, the compound of Formula I, the deuterated compound of Formula I, or the pharmaceutically acceptable salt thereof, within 24 hours before the viral exposure, then daily administration during the period of exposure, followed by a last administration approximately 24 hours later after the last exposure (which may be an increased dose, such as a double dose).

The specific dose level of a compound described herein for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

The daily dosage may also be described as a total amount of a compound described herein administered per dose or per day. Daily dosage of the compound of Formula A, the deuterated compound of Formula A, the prodrug of the compound of Formula A, the prodrug of the deuterated compound of Formula A, the compound of Formula I, the deuterated compound of Formula I, or the pharmaceutically acceptable salt thereof, may be between about 1 mg and 4,000 mg, between about 2,000 to 4,000 mg/day, between about 1 to 2,000 mg/day, between about 1 to 1,000 mg/day, between about 10 to 500 mg/day, between about 20 to 500 mg/day, between about 50 to 300 mg/day, between about 75 to 200 mg/day, or between about 15 to 150 mg/day.

The dosage or dosing frequency of a compound described herein may be adjusted over the course of the treatment, based on the judgment of the administering physician.

The compounds of the present disclosure may be administered to an individual (e.g., a human) in a therapeutically effective amount. In some embodiments, the compound is administered once daily. In some embodiments, the compound is administered twice daily.

The compounds described herein can be administered by any useful route and means, such as by oral or parenteral (e.g., intravenous) administration. Therapeutically effective amounts of the compound may include from about 0.00001 mg/kg body weight per day to about 10 mg/kg body weight per day, such as from about 0.0001 mg/kg body weight per day to about 10 mg/kg body weight per day, or such as from about 0.001 mg/kg body weight per day to about 1 mg/kg body weight per day, or such as from about 0.01 mg/kg body weight per day to about 1 mg/kg body weight per day, or such as from about 0.05 mg/kg body weight per day to about 0.5 mg/kg body weight per day. In some embodiments, a therapeutically effective amount of the compounds described herein include from about 0.3 mg to about 30 mg per day, or from about 30 mg to about 300 mg per day, or from about 0.3 mg to about 30 mg per day, or from about 30 mg to about 300 mg per day.

A compound described herein may be combined with one or more additional therapeutic agents in any dosage amount of the compound described herein (e.g., from 1 mg to 1000 mg of compound). Therapeutically effective amounts may include from about 0.1 mg per dose to about 1000 mg per dose, such as from about 50 mg per dose to about 500 mg per dose, or such as from about 100 mg per dose to about 400 mg per dose, or such as from about 150 mg per dose to about 350 mg per dose, or such as from about 200 mg per dose to about 300 mg per dose, or such as from about 0.01 mg per dose to about 1000 mg per dose, or such as from about 0.01 mg per dose to about 100 mg per dose, or such as from about 0.1 mg per dose to about 100 mg per dose, or such as from about 1 mg per dose to about 100 mg per dose, or such as from about 1 mg per dose to about 10 mg per dose, or such as from about 1 mg per dose to about 1000 mg per dose. Other therapeutically effective amounts of the compound of Formula A, the deuterated compound of Formula A, the prodrug of the compound of Formula A, the prodrug of the deuterated compound of Formula A, the compound of Formula I, the deuterated compound of Formula I, or the pharmaceutically acceptable salt thereof are about 1 mg per dose, or about 2, 3, 4, 5, 6, 7, 8, 9, 10, 15, 20, 25, 30, 35, 40, 45, 50, 55, 60, 65, 70, 75, 80, 85, 90, 95, or about 100 mg per dose. Other therapeutically effective amounts of the compound described herein are about 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or about 1000 mg per dose.

In some embodiments, the methods described herein comprise administering to the subject an initial daily dose of about 1 to 500 mg of a compound described herein and increasing the dose by increments until clinical efficacy is achieved. Increments of about 5, 10, 25, 50, or 100 mg can be used to increase the dose. The dosage can be increased daily, every other day, twice per week, once per week, once every two weeks, once every three weeks, or once a month.

When administered orally, the total daily dosage for a human subject may be between about 1-4,000 mg/day, between about 1-3,000 mg/day, between 1-2,000 mg/day, about 1-1,000 mg/day, between about 10-500 mg/day, between about 50-300 mg/day, between about 75-200 mg/day, or between about 100-150 mg/day. In some embodiments, the total daily dosage for a human subject may be about 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, or 3000 mg/day administered in a single dose. In some embodiments, the total daily dosage for a human subject may be about 200, 300, 400, 500, 600, 700, or 800 mg/day administered in a single dose. In some embodiments, the total daily dosage for a human subject may be about 300, 400, 500, or 600 mg/day administered in a single dose. In some embodiments, the total daily dosage for a human subject may be about 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, or 4000 mg/day. In some embodiments, the total daily dosage for a human subject may be about 100-200, 100-300, 100-400, 100-500, 100-600, 100-700, 100-800, 100-900, 100-1000, 500-1100, 500-1200, 500-1300, 500-1400, 500-1500, 500-1600, 500-1700, 500-1800, 500-1900, 500-2000, 1500-2100, 1500-2200, 1500-2300, 1500-2400, 1500-2500, 2000-2600, 2000-2700, 2000-2800, 2000-2900, 2000-3000, 2500-3100, 2500-3200, 2500-3300, 2500-3400, 2500-3500, 3000-3600, 3000-3700, 3000-3800, 3000-3900, or 3000-4000 mg/day.

In some embodiments, the total daily dosage for a human subject may be about 100 mg/day administered in a single dose. In some embodiments, the total daily dosage for a human subject may be about 150 mg/day administered in a single dose. In some embodiments, the total daily dosage for a human subject may be about 200 mg/day administered in a single dose. In some embodiments, the total daily dosage for a human subject may be about 250 mg/day administered in a single dose. In some embodiments, the total daily dosage for a human subject may be about 300 mg/day administered in a single dose. In some embodiments, the total daily dosage for a human subject may be about 350 mg/day administered in a single dose. In some embodiments, the total daily dosage for a human subject may be about 400 mg/day administered in a single dose. In some embodiments, the total daily dosage for a human subject may be about 450 mg/day administered in a single dose. In some embodiments, the total daily dosage for a human subject may be about 500 mg/day administered in a single dose. In some embodiments, the total daily dosage for a human subject may be about 550 mg/day administered in a single dose. In some embodiments, the total daily dosage for a human subject may be about 600 mg/day administered in a single dose. In some embodiments, the total daily dosage for a human subject may be about 650 mg/day administered in a single dose. In some embodiments, the total daily dosage for a human subject may be about 700 mg/day administered in a single dose. In some embodiments, the total daily dosage for a human subject may be about 750 mg/day administered in a single dose. In some embodiments, the total daily dosage for a human subject may be about 800 mg/day administered in a single dose. In some embodiments, the total daily dosage for a human subject may be about 850 mg/day administered in a single dose. In some embodiments, the total daily dosage for a human subject may be about 900 mg/day administered in a single dose. In some embodiments, the total daily dosage for a human subject may be about 950 mg/day administered in a single dose. In some embodiments, the total daily dosage for a human subject may be about 1000 mg/day administered in a single dose. In some embodiments, the total daily dosage for a human subject may be about 1500 mg/day administered in a single dose. In some embodiments, the total daily dosage for a human subject may be about 2000 mg/day administered in a single dose. In some embodiments, the total daily dosage for a human subject may be about 2500 mg/day administered in a single dose. In some embodiments, the total daily dosage for a human subject may be about 3000 mg/day administered in a single dose. In some embodiments, the total daily dosage for a human subject may be about 4000 mg/day administered in a single dose.

A single dose can be administered hourly, daily, weekly, or monthly. For example, a single dose can be administered once every 1 hour, 2, 3, 4, 6, 8, 12, 16 or once every 24 hours. A single dose can also be administered once every 1 day, 2, 3, 4, 5, 6, or once every 7 days. A single dose can also be administered once every 1 week, 2, 3, or once every 4 weeks. In certain embodiments, a single dose can be administered once every week. A single dose can also be administered once every month. In some embodiments, a compound described herein is administered once daily in a method described herein. In some embodiments, a compound described herein is administered twice daily in a method described herein. In some embodiments, a compound described herein is administered three times daily in a method described herein.

In some embodiments, a compound described herein is administered once daily in the total daily dose of 100-4000 mg/day. In some embodiments, a compound described herein is administered twice daily in the total daily dose of 100-4000 mg/day. In some embodiments, a compound described herein is administered three times daily in the total daily dose of 100-4000 mg/day.

The frequency of dosage of the compound described herein will be determined by the needs of the individual patient and can be, for example, once per day or twice, or more times, per day. Administration of the compound continues for as long as necessary to treat the viral infection. For example, a compound can be administered to a human being infected with the virus for a period of from 20 days to 180 days or, for example, for a period of from 20 days to 90 days or, for example, for a period of from 30 days to 60 days.

Administration can be intermittent, with a period of several or more days during which a patient receives a daily dose of the compound described herein followed by a period of several or more days during which a patient does not receive a daily dose of the compound. For example, a patient can receive a dose of the compound every other day, or three times per week. Again by way of example, a patient can receive a dose of the compound each day for a period of from 1 to 14 days, followed by a period of 7 to 21 days during which the patient does not receive a dose of the compound, followed by a subsequent period (e.g., from 1 to 14 days) during which the patient again receives a daily dose of the compound. Alternating periods of administration of the compound, followed by non-administration of the compound, can be repeated as clinically required to treat the patient.

The compounds of the present disclosure or the pharmaceutical compositions thereof may be administered once, twice, three, or four times daily, using any suitable mode described above. Also, administration or treatment with the compounds may be continued for a number of days; for example, commonly treatment would continue for at least 7 days, 14 days, or 28 days, for one cycle of treatment. Treatment cycles are well known in cancer chemotherapy, and are frequently alternated with resting periods of about 1 to 28 days, commonly about 7 days or about 14 days, between cycles. The treatment cycles, in other embodiments, may also be continuous.

In some embodiments, the compound is administered for 1 to 30 consecutive days, for example, 1 to 28 consecutive days, 1 to 21 consecutive days, 1 to 14 consecutive days, 1 to 7 consecutive days, 1 to 5 consecutive days, 3 to 30 consecutive days, 3 to 28 consecutive days, 3 to 21 consecutive days, 3 to 14 consecutive days, 3 to 7 consecutive days, 5 to 30 consecutive days, 5 to 28 consecutive days, 5 to 21 consecutive days, 5 to 14 consecutive days, or 5 to 7 consecutive days. In some embodiments, the compound is administered once daily or twice daily. In some embodiments, the compound is administered once daily. In some embodiments, the compound is administered twice daily.

In some embodiments, the compound is administered once daily for 2 consecutive days, 3 consecutive days, 4 consecutive days, 5 consecutive days, 6 consecutive days, 7 consecutive days, 8 consecutive days, 9 consecutive days, 10 consecutive days, 11 consecutive days, 12 consecutive days, 13 consecutive days, or 14 consecutive days. In some embodiments, the compound is administered once daily for 3 consecutive days. In some embodiments, the compound is administered once daily for 5 consecutive days. In some embodiments, the compound is administered twice daily for 2 consecutive days, 3 consecutive days, 4 consecutive days, 5 consecutive days, 6 consecutive days, 7 consecutive days, 8 consecutive days, 9 consecutive days, 10 consecutive days, 11 consecutive days, 12 consecutive days, 13 consecutive days, or 14 consecutive days. In some embodiments, the compound is administered twice daily for 3 consecutive days. In some embodiments, the compound is administered twice daily for 5 consecutive days.

In some embodiments, the compound described herein is administered to the human via oral, intramuscular, intravenous, subcutaneous, or inhalation administration. In some embodiments, the compound is administered orally.

In some embodiments, the patient is not pregnant on the first day of the administration of the compound of Formula A, the deuterated compound of Formula A, the prodrug of the compound of Formula A, the prodrug of the deuterated compound of Formula A, the compound of Formula I, the deuterated compound of Formula I, or the pharmaceutically acceptable salt thereof, and the patient avoids becoming pregnant from the first day to at least 1 day, at least 2 days, at least 3 days, at least 4 days, at least 5 days, at least 6 days, at least 7 days, at least 1 week, at least 2 weeks, at least 3 weeks, at least 4 weeks, or at least 1 month after the administration. In some embodiments, the patient is not pregnant on the first day of the administration of the compound of Formula A, the deuterated compound of Formula A, the prodrug of the compound of Formula A, the prodrug of the deuterated compound of Formula A, the compound of Formula I, the deuterated compound of Formula I, or the pharmaceutically acceptable salt thereof, and the patient avoids becoming pregnant from the first day to at least 2 days after the administration. In some embodiments, the patient is not pregnant on the first day of the administration of the compound of Formula A, the deuterated compound of Formula A, the prodrug of the compound of Formula A, the prodrug of the deuterated compound of Formula A, the compound of Formula I, the deuterated compound of Formula I, or the pharmaceutically acceptable salt thereof, and the patient avoids becoming pregnant from the first day to at least 4 days after the administration. In some embodiments, the patient is not pregnant on the first day of the administration of the compound of Formula A, the deuterated compound of Formula A, the prodrug of the compound of Formula A, the prodrug of the deuterated compound of Formula A, the compound of Formula I, the deuterated compound of Formula I, or the pharmaceutically acceptable salt thereof, and the patient avoids becoming pregnant from the first day to at least 7 days after the administration. In some embodiments, the patient is not pregnant on the first day of the administration of the compound of Formula A, the deuterated compound of Formula A, the prodrug of the compound of Formula A, the prodrug of the deuterated compound of Formula A, the compound of Formula I, the deuterated compound of Formula I, or the pharmaceutically acceptable salt thereof, and the patient avoids becoming pregnant from the first day to at least 14 days after the administration. In some embodiments, the patient is not pregnant on the first day of the administration of the compound of Formula A, the deuterated compound of Formula A, the prodrug of the compound of Formula A, the prodrug of the deuterated compound of Formula A, the compound of Formula I, the deuterated compound of Formula I, or the pharmaceutically acceptable salt thereof, and the patient avoids becoming pregnant from the first day to at least 1 month after the administration.

In some embodiments, the patient is a lactating individual, and the patient avoids breastfeeding from the first day of the administration of the compound of Formula A, the deuterated compound of Formula A, the prodrug of the compound of Formula A, the prodrug of the deuterated compound of Formula A, the compound of Formula I, the deuterated compound of Formula I, or the pharmaceutically acceptable salt thereof to at least 1 day, at least 2 days, at least 3 days, at least 4 days, at least 5 days, at least 6 days, at least 7 days, at least 1 week, at least 2 weeks, at least 3 weeks, at least 4 weeks, or at least 1 month after the administration. In some embodiments, the patient is a lactating individual, and the patient avoids breastfeeding from the first day of the administration of the compound of Formula A, the deuterated compound of Formula A, the prodrug of the compound of Formula A, the prodrug of the deuterated compound of Formula A, the compound of Formula I, the deuterated compound of Formula I, or the pharmaceutically acceptable salt thereof to at least 2 days after the administration. In some embodiments, the patient is a lactating individual, and the patient avoids breastfeeding from the first day of the administration of the compound of Formula A, the deuterated compound of Formula A, the prodrug of the compound of Formula A, the prodrug of the deuterated compound of Formula A, the compound of Formula I, the deuterated compound of Formula I, or the pharmaceutically acceptable salt thereof to at least 4 days after the administration. In some embodiments, the patient is a lactating individual, and the patient avoids breastfeeding from the first day of the administration of the compound of Formula A, the deuterated compound of Formula A, the prodrug of the compound of Formula A, the prodrug of the deuterated compound of Formula A, the compound of Formula I, the deuterated compound of Formula I, or the pharmaceutically acceptable salt thereof to at least 7 days after the administration. In some embodiments, the patient is a lactating individual, and the patient avoids breastfeeding from the first day of the administration of the compound of Formula A, the deuterated compound of Formula A, the prodrug of the compound of Formula A, the prodrug of the deuterated compound of Formula A, the compound of Formula I, the deuterated compound of Formula I, or the pharmaceutically acceptable salt thereof to at least 14 days after the administration. In some embodiments, the patient is a lactating individual, and the patient avoids breastfeeding from the first day of the administration of the compound of Formula A, the deuterated compound of Formula A, the prodrug of the compound of Formula A, the prodrug of the deuterated compound of Formula A, the compound of Formula I, the deuterated compound of Formula I, or the pharmaceutically acceptable salt thereof to at least 1 month after the administration.

In some embodiments, the patient is not a lactating individual.

In some embodiments, the method further comprises determining that the patient is not pregnant before administering the compound of Formula A, the deuterated compound of Formula A, the prodrug of the compound of Formula A, the prodrug of the deuterated compound of Formula A, the compound of Formula I, the deuterated compound of Formula I, or the pharmaceutically acceptable salt thereof. In some embodiments, the determining comprises classifying the patient as potentially child-bearing or not potentially child-bearing, and administering a pregnancy test to a potentially child-bearing patient.

In some embodiments, the patient is potentially child-bearing, and the patient has had at least one negative pregnancy test at least one day prior to the administration of the compound of Formula A, the deuterated compound of Formula A, the prodrug of the compound of Formula A, the prodrug of the deuterated compound of Formula A, the compound of Formula I, the deuterated compound of Formula I, or the pharmaceutically acceptable salt thereof. In some embodiments, the patient is potentially child-bearing, and the patient has had at least two negative pregnancy tests, each individually at least one day prior to the administration of the compound of Formula A, the deuterated compound of Formula A, the prodrug of the compound of Formula A, the prodrug of the deuterated compound of Formula A, the compound of Formula I, the deuterated compound of Formula I, or the pharmaceutically acceptable salt thereof.

In some embodiments, the patient has had a first negative pregnancy test at least 7 days, at least 8 days, at least 9 days, at least 10 days, at least 11 days, at least 12 days, at least 13 days, at least 14 days, at least 3 weeks, or at least 4 weeks prior to the administration. In some embodiments, the patient has had a second negative pregnancy test after the first pregnancy test and at least one day prior to the administration. In some embodiments, the patient has had a first negative pregnancy test at least 7 days prior to the administration and a second negative pregnancy test after the first pregnancy test and at least one day prior to the administration. In some embodiments, the patient has had a first negative pregnancy test at least 14 days prior to the administration and a second negative pregnancy after the first pregnancy test and at least one day prior to the administration.

In some embodiments, the patient has had the second negative pregnancy test during the first 10 days, the first 9 days, the first 8 days, the first 7 days, the first 6 days, the first 5 days, the first 4 days, or the first 3 days of the menstrual period immediately preceding the administration. In some embodiments, the patient has had the second negative pregnancy test during the first 5 days of the menstrual period immediately preceding the administration. In some embodiments, the patient has amenorrhea, and the patient has had the second negative pregnancy test at least 5 days, at least 6 days, at least 7 days, at least 8 days, at least 9 days, at least 10 days, at least 11 days, at least 12 days, at least 13 days, or at least 14 days after the last act of unprotected sexual intercourse. In some embodiments, the patient has amenhorrhea, and the patient has had the second negative pregnancy test at least 11 days after the last act of unprotected sexual intercourse.

In some embodiments, each pregnancy test is independently a urine pregnancy test or a serum pregnancy test. In some embodiments, each pregnancy test independently has a sensitivity of at least 10 mIU/mL, at least 15 mIU/mL, at least 20 mIU/mL, or at least 25 mIU/mL. In some embodiments, each pregnancy test independently has a sensitivity of at least 25 mIU/mL.

In some embodiments, the patient is potentially child-bearing, and the patient uses at least one form of contraception from the first day of the administration of the compound of Formula A, the deuterated compound of Formula A, the prodrug of the compound of Formula A, the prodrug of the deuterated compound of Formula A, the compound of Formula I, the deuterated compound of Formula I, or the pharmaceutically acceptable salt thereof, to at least 1 day, at least 2 days, at least 3 days, at least 4 days, at least 5 days, at least 6 days, at least 7 days, at least 1 week, at least 2 weeks, at least 3 weeks, at least 4 weeks, or at least 1 month after the administration. In some embodiments, the patient is potentially child-bearing, and the patient uses at least two forms of contraception from the first day of the administration of the compound of Formula A, the deuterated compound of Formula A, the prodrug of the compound of Formula A, the prodrug of the deuterated compound of Formula A, the compound of Formula I, the deuterated compound of Formula I, or the pharmaceutically acceptable salt thereof, to at least 1 day, at least 2 days, at least 3 days, at least 4 days, at least 5 days, at least 6 days, at least 7 days, at least 1 week, at least 2 weeks, at least 3 weeks, at least 4 weeks, or at least 1 month after the administration.

In some embodiment, the patient uses the contraception from at least one week, at least two weeks, at least three weeks, at least four weeks, or at least one month prior to the first day of the administration. In some embodiments, the patient uses the contraception from at least 1 month prior to the first day of the administration. In some embodiments, the patient uses the contraception until at least one week, at least two weeks, at least three weeks, at least four weeks, or at least one month after the administration. In some embodiments, the patient uses the contraception until at least one month after the administration.

In some embodiments, the patient uses at least one form of contraception selected from tubal ligation, partner's vasectomy, intrauterine devices, birth control pills, and injectable, implantable, and insertable hormonal birth control products. In some embodiments, the patient uses at least one form of contraception selected from diaphragms, latex condoms, and cervical caps. In some embodiments, the diaphragms, latex condoms, and cervical caps are used with a spermicide.

In some embodiments, the patient is a high-risk patient. For example, the patient is high risk for progression to severe stage of a viral infection such as to severe COVID-19, including hospitalization of death. Risk factors for progression to hospitalization included e.g., age≥60 years, obesity (BMI≥30), chronic lung disease, hypertension, cardiovascular or cerebrovascular disease, diabetes mellitus, immunocompromised state, chronic mild or moderate kidney disease, chronic liver disease, current cancer, and sickle cell disease. In some embodiments, the high-risk patient is age 50 years or older. In some embodiments, the high-risk patient is age 65 years or older. In some embodiments, the high-risk patient is immunocompromised. In some embodiments, the high-risk patient has a weakened immune system. In some embodiments, the high-risk patient is taking medicine that weakens the immune system. In some embodiments, the high-risk patient has obesity. In some embodiments, the high-risk patient has chronic obstructive pulmonary disorder. In some embodiments, the high-risk patient has severe heart disease. In some embodiments, the high-risk patient has high blood pressure.

In some embodiments, the present disclosure provides for methods of treating or preventing a viral infection in a subject (e.g., human) in need thereof, the method comprising administering to the subject a compound described herein and at least one additional active therapeutic or prophylactic agent.

In some embodiments, the present disclosure provides for methods of treating a viral infection in a subject (e.g., human) in need thereof, the method comprising administering to the subject a compound described herein, and at least one additional active therapeutic or prophylactic agent.

In one embodiment, the present disclosure provides for methods of inhibiting a viral polymerase in a cell, the methods comprising contacting the cell infected a virus with a compound described herein, whereby the viral polymerase is inhibited.

In one embodiment, the present disclosure provides for methods of inhibiting a viral polymerase in a cell, the methods comprising contacting the cell infected a virus with a compound described herein, and at least one additional active therapeutic agent, whereby the viral polymerase is inhibited.

Also provided here are the uses of the compounds described herein for use in treating or preventing a viral infection in a subject in need thereof. For example, provided herein are uses of the compounds described herein for use in treating a viral infection in a subject in need thereof.

In some embodiments, the viral infection is aparamyxoviridae virus infection. As such, in some embodiments, the present disclosure provides methods for treating a paramyxoviridae infection in a subject (e.g., a human) in need thereof, the method comprising administering to the subject a compound described herein. Paramyxoviridae viruses include, but are not limited to Nipah virus, Hendra virus, measles, mumps, and parainfluenze virus.

In some embodiments, the viral infection is a human parainfluenza virus, Nipah virus, Hendra virus, measles, or mumps infection.

In some embodiments, the viral infection is apneumoviridae virus infection. As such, in some embodiments, the present disclosure provides a method of treating a pneumoviridae virus infection in a human in need thereof, the method comprising administering to the human a compound described herein. Pneumoviridae viruses include, but are not limited to, respiratory snycytial virus and human metapneumovirus. In some embodiments, the pneumoviridae virus infection is a respiratory syncytial virus infection. In some embodiments, the pneumoviridae virus infection is human metapneumovirus infection.

In some embodiments, the present disclosure provides a compound described herein, for use in the treatment of a pneumoviridae virus infection in a human in need thereof. In some embodiments, the pneumoviridae virus infection is a respiratory syncytial virus infection. In some embodiments, the pneumoviridae virus infection is human metapneumovirus infection.

In some embodiments, the present disclosure provides methods for treating a RSV infection in a human in need thereof, the method comprising administering to the human a compound described herein. In some embodiments, the human is suffering from a chronic respiratory syncytial viral infection. In some embodiments, the human is acutely infected with RSV.

In some embodiments, a method of inhibiting RSV replication is provided, wherein the method comprises administering to a human in need thereof, a compound described herein, wherein the administration is by inhalation.

In some embodiments, the present disclosure provides a method for reducing the viral load associated with RSV infection, wherein the method comprises administering to a human infected with RSV a compound described herein.

In some embodiments, the viral infection is apicornaviridae virus infection. As such, in some embodiments, the present disclosure provides a method of treating a picornaviridae virus infection in a human in need thereof, the method comprising administering to the human a compound described herein. Picornaviridae viruses are eneteroviruses causing a heterogeneous group of infections including herpangina, aseptic meningitis, a common-cold-like syndrome (human rhinovirus infection), a non-paralytic poliomyelitis-like syndrome, epidemic pleurodynia (an acute, febrile, infectious disease generally occurring in epidemics), hand-foot-mouth syndrome, pediatric and adult pancreatitis and serious myocarditis. In some embodiments, the picornaviridae virus infection is human rhinovirus infection (HRV). In some embodiments, the picornaviridae virus infection is HRV-A, HRV-B, or HRV-C infection.

In some embodiments, the viral infection is selected from Coxsackie A virus infection, Coxsackie A virus infection, enterovirus D68 infection, enterovirus B69 infection, enterovirus D70 infection, enterovirus A71 infection, and poliovirus infection.

In some embodiments, the present disclosure provides a compound, for use in the treatment of a picornaviridae virus infection in a human in need thereof. In some embodiments, the picornaviridae virus infection is human rhinovirus infection.

In some embodiments, the viral infection is a flaviviridae virus infection. As such, in some embodiments, the present disclosure provides a method of treating a flaviviridae virus infection in a human in need thereof, the method comprising administering to the human a compound described herein. Representativeflaviviridae viruses include, but are not limited to, dengue, Yellow fever, West Nile, Zika, Japanese encephalitis virus, and Hepatitis C (HCV). In some embodiments, the flaviviridae virus infection is a dengue virus infection. In some embodiments, the flaviviridae virus infection is a yellow fever virus infection. In some embodiments, the flaviviridae virus infection is a West Nile virus infection. In some embodiments, the flaviviridae virus infection is a zika virus infection. In some embodiments, the flaviviridae virus infection is a Japanese ensephalitis virus infection. In some embodiments, the flaviviridae virus infection is a hepatitis C virus infection.

In some embodiments, the flaviviridae virus infection is a dengue virus infection, yellow fever virus infection, West Nile virus infection, tick borne encephalitis, Kunjin Japanese encephalitis, St. Louis encephalitis, Murray valley encephalitis, Omsk hemorrhagic fever, bovine viral diarrhea, zika virus infection, or a HCV infection.

In some embodiments, the present disclosure provides use of a compound described herein for treatment of a flaviviridae virus infection in a human in need thereof. In some embodiments, the flaviviridae virus infection is a dengue virus infection. In some embodiments, the flaviviridae virus infection is a yellow fever virus infection. In some embodiments, the flaviviridae virus infection is a West Nile virus infection. In some embodiments, the flaviviridae virus infection is a zika virus infection. In some embodiments, the flaviviridae virus infection is a hepatitis C virus infection.

In some embodiments, the viral infection is a filoviridae virus infection. As such, in some embodiments, provided herein is a method of treating a filoviridae virus infection in a human in need thereof, the method comprising administering to the human a compound described herein. Representative filoviridae viruses include, but are not limited to, ebola (variants Zaire, Bundibugio, Sudan, Tai forest, or Reston) and marburg. In some embodiments, the filoviridae virus infection is an ebola virus infection. In some embodiments, the filoviridae virus infection is a marburg virus infection.

In some embodiments, the present disclosure provides a compound for use in the treatment of a floviridae virus infection in a human in need thereof. In some embodiments, the filoviridae virus infection is an ebola virus infection. In some embodiments, the filoviridae virus infection is a marburg virus infection.

In some embodiments, the viral infection is a coronavirus infection. As such, in some embodiments, provided herein is a method of treating a coronavirus infection in a human in need thereof, wherein the method comprises administering to the human a compound described herein. In some embodiments, the coronavirus infection is a Severe Acute Respiratory Syndrome (SARS-CoV) infection, Middle Eastern Respiratory Syndrome (MERS) infection, SARS-CoV-2 infection, other human coronavirus (229E, NL63, OC43, HKU1, or WIV1) infections, zoonotic coronavirus (PEDV or HKU CoV isolates such as HKU3, HKU5, or HKU9) infections. In some embodiments, the viral infection is a Severe Acute Respiratory Syndrome (SARS) infection. In some embodiments, the viral infection is a Middle Eastern Respiratory Syndrome (MERS) infection. In some embodiments, the viral infection is SARS-CoV-2 infection. In some embodiments, the viral infection is a zoonotic coronavirus infection, In some embodiments, the viral infection is caused by a virus having at least 70% sequence homology to a viral polymerase selected from SARS-CoV polymerase, MERS-CoV polymerase and SARS-CoV-2. In some embodiments, the viral infection is caused by a virus having at least 80% sequence homology to a viral polymerase selected from SARS-CoV polymerase, MERS-CoV polymerase and SARS-CoV-2. In some embodiments, the viral infection is caused by a virus having at least 90% sequence homology to a viral polymerase selected from SARS-CoV polymerase, MERS-CoV polymerase and SARS-CoV-2. In some embodiments, the viral infection is caused by a virus having at least 95% sequence homology to a viral polymerase selected from SARS-CoV polymerase, MERS-CoV polymerase and SARS-CoV-2.

In some embodiments, the viral infection is caused by a variant of SARS-CoV-2, for example by the B.1.1.7 variant (the UK variant), B.1.351 variant (the South African variant), P.1 variant (the Brazil variant), B.1.1.7 with E484K variant, B.1.1.207 variant, B.1.1.317 variant, B.1.1.318 variant, B.1.429 variant, B.1.525 variant, or P.3 variant. In some embodiments, the viral infection is caused by the B.1.1.7 variant of SARS-CoV-2. In some embodiments, the viral infection is caused by the B.1.351 variant of SARS-CoV-2. In some embodiments, the viral infection is caused by the P.1 variant of SARS-CoV-2.

In some embodiments, the present disclosure provides a compound for use in the treatment of a coronavirus virus infection in a human in need thereof. In some embodiments, the coronavirus infection is a Severe Acute Respiratory Syndrome (SARS) infection, Middle Eastern Respiratory Syndrome (MERS) infection, SARS-CoV-2 infection, other human coronavirus (229E, NL63, OC43, HKU1, or WIV1) infections, and zoonotic coronavirus (PEDV or HKU CoV isolates such as HKU3, HKU5, or HKU9) infections. In some embodiments, the viral infection is a Severe Acute Respiratory Syndrome (SARS) infection. In some embodiments, the viral infection is a Middle Eastern Respiratory Syndrome (MERS) infection. In some embodiments, the viral infection is SARS-CoV-2 infection (COVID19).

In some embodiments, the viral infection is an arenaviridae virus infection. As such, in some embodiments, the disclosure provides a method of treating an arenaviridae virus infection in a human in need thereof, the method comprising administering to the human a compound described herein. In some embodiments, the arenaviridae virus infection is a Lassa infection or a Junin infection.

In some embodiments, the present disclosure provides a compound for use in the treatment of an arenaviridae virus infection in a human in need thereof. In some embodiments, the arenaviridae virus infection is a Lassa infection or a Junin infection.

In some embodiments, the viral infection is an orthomyxovirus infection, for example, an influenza virus infection. In some embodiments, the viral infection is an influenza virus A, influenza virus B, or influenza virus C infection.

As described more fully herein, the compounds described herein can be administered with one or more additional therapeutic agent(s) to an individual (e.g., a human) infected with a viral infection. The additional therapeutic agent(s) can be administered to the infected individual at the same time as the compound described herein or before or after administration of the compound described herein.

The compounds described herein can also be used in combination with one or more additional therapeutic agents. As such, also provided herein are methods of treatment of a viral infection in a subject in need thereof, wherein the methods comprise administering to the subject a compound disclosed therein and a therapeutically effective amount of one or more additional therapeutic or prophylactic agents.

In some embodiments, the additional therapeutic agent is an antiviral agent. Any suitable antiviral agent can be used in the methods described herein. In some embodiments, the antiviral agent is selected from 5-substituted 2′-deoxyuridine analogues, nucleoside analogues, pyrophosphate analogues, nucleoside reverse transcriptase inhibitors, non-nucleoside reverse transcriptase inhibitors, protease inhibitors, integrase inhibitors, entry inhibitors, acyclic guanosine analogues, acyclic nucleoside phosphonate analogues, HCV NS5A/NS5B inhibitors, influenza virus inhibitors, interferons, immunostimulators, oligonucleotides, antimitotic inhibitors, and combinations thereof.

In some embodiments, the additional therapeutic agent is a 5-substituted 2′-deoxyuridine analogue. For example, in some embodiments, the additional therapeutic agent is selected from idoxuridine, trifluridine, brivudine [BVDU], and combinations thereof.

In some embodiments, the additional therapeutic agent is a nucleoside analogue. For example, in some embodiments, the additional therapeutic agent is selected from vidarabine, entecavir (ETV), telbivudine, lamivudine, adefovir dipivoxil, tenofovir disoproxil fumarate (TDF) and combinations thereof. In some embodiments, the additional therapeutic agent is favipiravir, ribavirin, galidesivir, 3-D-N4-hydroxycytidine or a combination thereof.

In some embodiments, the additional therapeutic agent is a pyrophosphate analogue. For example, in some embodiments, the additional therapeutic agent is foscarnet or phosphonoacetic acid. In some embodiments, the additional therapeutic agent is foscarnet.

In some embodiments, the additional therapeutic agent is nucleoside reverse transcriptase inhibitor. In some embodiments, the antiviral agent is zidovudine, didanosine, zalcitabine, stavudine, lamivudine, abacavir, emtricitabine, and combinations thereof.

In some embodiments, the additional therapeutic agent is a non-nucleoside reverse transcriptase inhibitor. In some embodiments, the antiviral agent is selected from nevirapine, delavirdine, efavirenz, etravirine, rilpivirine, and combinations thereof.

In some embodiments, the additional therapeutic agent is a protease inhibitor. In some embodiments, the protease inhibitor is a HIV protease inhibitor. For example, in some embodiments, the antiviral agent is selected from saquinavir, ritonavir, indinavir, nelfinavir, amprenavir, lopinavir, atazanavir, fosamprenavir, darunavir, tipranavir, cobicistat, and combinations thereof. In some embodiments, the antiviral agent is selected from saquinavir, ritonavir, indinavir, nelfinavir, amprenavir, lopinavir, atazanavir, fosamprenavir, darunavir, tipranavir, and combinations thereof. In some embodiments, the protease inhibitor is a HCV NS3/4A protease inhibitor. For example, in some embodiments, the additional therapeutic agent is selected from voxilaprevir, asunaprevir, boceprevir, paritaprevir, simeprevir, telaprevir, vaniprevir, grazoprevir, ribavirin, danoprevir, faldaprevir, vedroprevir, sovaprevir, deldeprevir, narlaprevir and combinations thereof. In some embodiments, the additional therapeutic agent is selected from voxilaprevir, asunaprevir, boceprevir, paritaprevir, simeprevir, telaprevir, vaniprevir, grazoprevir, and combinations thereof.

In some embodiments, the additional therapeutic agent is an integrase inhibitor. For example, in some embodiments, the additional therapeutic agent is selected from raltegravir, dolutegravir, elvitegravir, abacavir, lamivudine, and combinations thereof. In some embodiments, the additional therapeutic agent is selected from bictegravir, raltegravir, dolutegravir, cabotegravir, elvitegravir, and combinations thereof. In some embodiments, the additional therapeutic agent is selected from bictegravir, dolutegravir, and cabotegravir, and combinations thereof. In some embodiments, the additional therapeutic agent is bictegravir.

In some embodiments, the additional therapeutic agent is an entry inhibitor. For example, in some embodiments, the additional therapeutic agent is selected from docosanol, enfuvirtide, maraviroc, ibalizumab, fostemsavir, leronlimab, ibalizumab, fostemsavir, leronlimab, palivizumab, respiratory syncytial virus immune globulin, intravenous [RSV-IGIV], varicella-zoster immunoglobulin [VariZIG], varicella-zoster immune globulin [VZIG]), and combinations thereof.

In some embodiments, the additional therapeutic agent is an acyclic guanosine analogue. For example, in some embodiments, the additional therapeutic agent is selected from acyclovir, ganciclovir, valacyclovir (also known as valaciclovir), valganciclovir, penciclovir, famciclovir, and combinations thereof.

In some embodiments, the additional therapeutic agent is an acyclic nucleoside phosphonate analogue. For example, in some embodiments, the additional therapeutic agent is selected from a group consisting of cidofovir, adefovir, adefovir dipivoxil, tenofovir, TDF, emtricitabine, efavirenz, rilpivirine, elvitegravir, and combinations thereof. In some embodiment, the additional therapeutic agent is selected from cidofovir, adefovir, adefovir dipivoxil, tenofovir, TDF, and combinations thereof. In some embodiment, the additional therapeutic agent is selected from cidofovir, adefovir dipivoxil, TDF, and combinations thereof.

In some embodiments, the additional therapeutic agent is a HCV NS5A/NS5B inhibitor. In some embodiments, the additional therapeutic agent is a NS3/4A protease inhibitor. In some embodiments, the additional therapeutic agent is a NS5A protein inhibitor. In some embodiments, the additional therapeutic agent is a NS5B polymerase inhibitor of the nucleoside/nucleotide type. In some embodiments, the additional therapeutic agent is a NS5B polymerase inhibitor of the nonnucleoside type. In some embodiments, the additional therapeutic agent is selected from daclatasvir, ledipasvir, velpatasvir, ombitasvir, elbasvir, sofosbuvir, dasabuvir, ribavirin, asunaprevir, simeprevir, paritaprevir, ritonavir, elbasvir, grazoprevir, AT-527, and combinations thereof. In some embodiments, the additional therapeutic agent is selected from daclatasvir, ledipasvir, velpatasvir, ombitasvir, elbasvir, sofosbuvir, dasabuvir, and combinations thereof.

In some embodiments, the additional therapeutic agent is an influenza virus inhibitor. In some embodiments, the additional therapeutic agent is a matrix 2 inhibitor. For example, in some embodiments, the additional therapeutic agent is selected from amantadine, rimantadine, and combinations thereof. In some embodiments, the additional therapeutic agent is a neuraminidase inhibitor. For example, in some embodiments, the additional therapeutic agent is selected from zanamivir, oseltamivir, peramivir, laninamivir octanoate, and combinations thereof. In some embodiments, the additional therapeutic agent is a polymerase inhibitor. For example, in some embodiments, the additional therapeutic agent is selected from ribavirin, favipiravir, and combinations thereof. In some embodiments, the additional therapeutic agent is selected from amantadine, rimantadine, arbidol (umifenovir), baloxavir marboxil, oseltamivir, peramivir, ingavirin, laninamivir octanoate, zanamivir, favipiravir, ribavirin, and combinations thereof. In some embodiments, the additional therapeutic agent is selected from amantadine, rimantadine, zanamivir, oseltamivir, peramivir, laninamivir octanoate, ribavirin, favipiravir, and combinations thereof.

In some embodiments, the additional therapeutic agent is an interferon. In some embodiments, the additional therapeutic agent is selected from interferon alfacon 1, interferon alfa 1b, interferon alfa 2a, interferon alfa 2b, pegylated interferon alfacon 1, pegylated interferon alfa 1b, pegylated interferon alfa 2a (PegIFNα-2a), and PegIFNα-2b. e embodiments, the additional therapeutic agent is selected from interferon alfacon 1, interferon alfa 1b, interferon alfa 2a, interferon alfa 2b, pegylated interferon alfa 2a (PegIFNα-2a), and PegIFNα-2b. In some embodiments, the additional therapeutic agent is selected from interferon alfacon 1, pegylated interferon alfa 2a (PegIFNα-2a), PegIFNα-2b, and ribavirin. In some embodiments, the additional therapeutic agent is pegylated interferon alfa-2a, pegylated interferon alfa-2b, or a combination thereof.

In some embodiments, the additional therapeutic agent is an immunostimulatory agent. In some embodiments, the additional therapeutic agent is an oligonucleotide. In some embodiments, the additional therapeutic agent is an antimitotic inhibitor. For example, in some embodiments, the additional therapeutic agent is selected from fomivirsen, podofilox, imiquimod, sinecatechins, and combinations thereof.

In some embodiments, the additional therapeutic agent is selected from besifovir, nitazoxanide, REGN2222, doravirine, sofosbuvir, velpatasvir, daclatasvir, asunaprevir, beclabuvir, FV100, and letermovir, and combinations thereof.

In some embodiments, the additional therapeutic agent is an agent for treatment of RSV. For example, in some embodiments, the antiviral agent is ribavirin, ALS-8112 or presatovir. For example, in some embodiments, the antiviral agent is ALS-8112 or presatovir.

In some embodiments, the additional therapeutic agent is an agent for treatment of picornavirus. In some embodiments, the additional therapeutic agent is selected from hydantoin, guanidine hydrochloride, 1-buthionine sulfoximine, Py-11, and combinations thereof. In some embodiments, the additional therapeutic agent is a picornavirus polymerase inhibitor. In some embodiments, the additional therapeutic agent is rupintrivir.

In some embodiments, the additional therapeutic agent is an agent for treatment of malaria. In some embodiments, the additional therapeutic agent is chloroquine.

In some embodiments, the additional therapeutic agent is selected from hydroxychloroquine, chloroquine, artemether, lumefantrine, atovaquone, proguanil, tafenoquine, pyronaridine, artesunate, artenimol, piperaquine, artesunate, amodiaquine, pyronaridine, artesunate, halofantrine, quinine sulfate, mefloquine, solithromycin, pyrimethamine, MMV-390048, ferroquine, artefenomel mesylate, ganaplacide, DSM-265, cipargamin, artemisone, and combinations thereof.

In some embodiments, the additional therapeutic agent is an agent for treatment of coronavirus. In some embodiments, the additional therapeutic agent is selected from a group consisting of IFX-1, FM-201, CYNK-001, DPP4-Fc, ranpirnase, nafamostat, LB-2, AM-1, anti-viroporins, and combinations thereof.

In some embodiments, the additional therapeutic agent is an agent for treatment of ebola virus. For example, in some embodiments, the additional therapeutic agent is selected from ribavirin, palivizumab, motavizumab, RSV-IGIV (RespiGam®), MEDI-557, A-60444, MDT-637, BMS-433771, amiodarone, dronedarone, verapamil, Ebola Convalescent Plasma (ECP), TKM-100201, BCX4430 ((2S,3S,4R,5R)-2-(4-amino-5H-pyrrolo[3,2-d]pyrimidin-7-yl)-5-(hydroxymethyl)pyrrolidine-3,4-diol), favipiravir (also known as T-705 or Avigan), T-705 monophosphate, T-705 diphosphate, T-705 triphosphate, FGI-106 (1-N,7-N-bis[3-(dimethylamino)propyl]-3,9-dimethylquinolino[8,7-h]quinolone-1,7-diamine), JK-05, TKM-Ebola, ZMapp, rNAPc2, VRC-EBOADC076-00-VP, OS-2966, MVA-BN filo, brincidofovir, Vaxart adenovirus vector 5-based ebola vaccine, Ad26-ZEBOV, FiloVax vaccine, GOVX-E301, GOVX-E302, ebola virus entry inhibitors (NPC1 inhibitors), rVSV-EBOV, and combinations thereof. In some embodiments, the additional therapeutic agent is ZMapp, mAB 114, REGEN-EB3, and combinations thereof.

In some embodiments, the additional therapeutic agent is an agent for treatment of HCV. In some embodiments, the additional therapeutic agent is a HCV polymerase inhibitor. For example, in some embodiments, the additional therapeutic agent is selected from sofosbuvir, GS-6620, PSI-938, ribavirin, tegobuvir, radalbuvir, MK-0608, and combinations thereof. In some embodiments, the additional therapeutic agent is a HCV protease inhibitor. For example, in some embodiments, the additional therapeutic agent is selected from such as GS-9256, vedroprevir, voxilaprevir, and combinations thereof.

In some embodiments, the additional therapeutic agent is a NS5A inhibitor. For example, in some embodiments, the additional therapeutic agent is selected from ledipasvir, velpatasvir, and combinations thereof.

In some embodiments, the additional therapeutic agent is an anti HBV agent. For example, in some embodiments, the additional therapeutic agent is tenofovir disoproxil fumarate and emtricitabine, or a combination thereof. Examples of additional anti HBV agents include but are not limited to alpha-hydroxytropolones, amdoxovir, antroquinonol, beta-hydroxycytosine nucleosides, ARB-199, CCC-0975, ccc-R08, elvucitabine, ezetimibe, cyclosporin A, gentiopicrin (gentiopicroside), HH-003, hepalatide, JNJ-56136379, nitazoxanide, birinapant, NJK14047, NOV-205 (molixan, BAM-205), oligotide, mivotilate, feron, GST-HG-131, levamisole, Ka Shu Ning, alloferon, WS-007, Y-101 (Ti Fen Tai), rSIFN-co, PEG-IIFNm, KW-3, BP-Inter-014, oleanolic acid, HepB-nRNA, cTP-5 (rTP-5), HSK-II-2, HEISCO-106-1, HEISCO-106, Hepbarna, IBPB-006IA, Hepuyinfen, DasKloster 0014-01, ISA-204, Jiangantai (Ganxikang), MIV-210, OB-AI-004, PF-06, picroside, DasKloster-0039, hepulantai, IMB-2613, TCM-800B, reduced glutathione, RO-6864018, RG-7834, QL-007 sofosbuvir, ledipasvir, UB-551, and ZH-2N, and the compounds disclosed in US20150210682, (Roche), US 2016/0122344 (Roche), WO2015173164, WO2016023877, US2015252057A (Roche), WO16128335A1 (Roche), WO16120186A1 (Roche), US2016237090A (Roche), WO16107833A1 (Roche), WO16107832A1 (Roche), US2016176899A (Roche), WO16102438A1 (Roche), WO16012470A1 (Roche), US2016220586A (Roche), and US2015031687A (Roche). In some embodiments, the additional therapeutic agent is a HBV polymerase inhibitor. Examples of HBV DNA polymerase inhibitors include, but are not limited to, adefovir (HEPSERA®), emtricitabine (EMTRIVA®), tenofovir disoproxil fumarate (VIREAD®), tenofovir alafenamide, tenofovir, tenofovir disoproxil, tenofovir alafenamide fumarate, tenofovir alafenamide hemifumarate, tenofovir dipivoxil, tenofovir dipivoxil fumarate, tenofovir octadecyloxyethyl ester, CMX-157, tenofovir exalidex, besifovir, entecavir (BARACLUDE®), entecavir maleate, telbivudine (TYZEKA®), filocilovir, pradefovir, clevudine, ribavirin, lamivudine (EPIVIR-HBV®), phosphazide, famciclovir, fusolin, metacavir, SNC-019754, FMCA, AGX-1009, AR-II-04-26, HIP-1302, tenofovir disoproxil aspartate, tenofovir disoproxil orotate, and HS-10234. In some embodiments, the additional therapeutic agent is a HBV capsid inhibitor.

In some embodiments, the additional therapeutic agent is an agent for treatment of HIV. In some embodiments, the additional therapeutic agent is selected from HIV protease inhibitors, HIV integrase inhibitors, entry inhibitors, HIV nucleoside reverse transcriptase inhibitors, HIV nonnucleoside reverse transcriptase inhibitors, acyclic nucleoside phosphonate analogues, and combinations thereof.

In some embodiments, the additional therapeutic agent is selected from HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, immunomodulators, immunotherapeutic agents, antibody-drug conjugates, gene modifiers, gene editors (such as CRISPR/Cas9, zinc finger nucleases, homing nucleases, synthetic nucleases, TALENs), and cell therapies (such as chimeric antigen receptor T-cell, CAR-T, and engineered T cell receptors, TCR-T, autologous T cell therapies).

In some embodiments, the additional therapeutic agent is selected from combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors, HIV antibodies, and bispecific antibodies, and “antibody-like” therapeutic proteins, and combinations thereof.

In some embodiments, the additional therapeutic agent is a HIV combination drug. Examples of the HIV combination drugs include, but are not limited to ATRIPLA® (efavirenz, tenofovir disoproxil fumarate, and emtricitabine); BIKTARVY® (bictegravir, emtricitabine, and tenofovir alafenamide); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); SYMTUZA® (darunavir, tenofovir alafenamide hemifumarate, emtricitabine, and cobicistat); SYMFI™ (efavirenz, lamivudine, and tenofovir disoproxil fumarate); CIMDU™ (lamivudine and tenofovir disoproxil fumarate); tenofovir and lamivudine; tenofovir alafenamide and emtricitabine; tenofovir alafenamide hemifumarate and emtricitabine; tenofovir alafenamide hemifumarate, emtricitabine, and rilpivirine; tenofovir alafenamide hemifumarate, emtricitabine, cobicistat, and elvitegravir; COMBIVIR® (zidovudine and lamivudine; AZT+3TC); EPZICOM® (LIVEXA®; abacavir sulfate and lamivudine; ABC+3TC); KALETRA® (ALUVIA®; lopinavir and ritonavir); TRIUMEQ® (dolutegravir, abacavir, and lamivudine); TRIZIVIR® (abacavir sulfate, zidovudine, and lamivudine; ABC+AZT+3TC); atazanavir and cobicistat; atazanavir sulfate and cobicistat; atazanavir sulfate and ritonavir; darunavir and cobicistat; dolutegravir and rilpivirine; dolutegravir and rilpivirine hydrochloride; dolutegravir, abacavir sulfate, and lamivudine; lamivudine, nevirapine, and zidovudine; raltegravir and lamivudine; doravirine, lamivudine, and tenofovir disoproxil fumarate; doravirine, lamivudine, and tenofovir disoproxil; dapivirine+levonorgestrel, dolutegravir+lamivudine, dolutegravir+emtricitabine+tenofovir alafenamide, elsulfavirine+emtricitabine+tenofovir disoproxil, lamivudine+abacavir+zidovudine, lamivudine+abacavir, lamivudine+tenofovir disoproxil fumarate, lamivudine+zidovudine+nevirapine, lopinavir+ritonavir, lopinavir+ritonavir+abacavir+lamivudine, lopinavir+ritonavir+zidovudine+lamivudine, tenofovir+lamivudine, and tenofovir disoproxil fumarate+emtricitabine+rilpivirine hydrochloride, lopinavir, ritonavir, zidovudine and lamivudine.

In some embodiments, the additional therapeutic agent is a HIV protease inhibitor. For example, in some embodiments the additional therapeutic agent is selected from saquinavir, ritonavir, indinavir, nelfinavir, amprenavir, lopinavir, atazanavir, fosamprenavir, darunavir, tipranavir, cobicistat, ASC-09, AEBL-2, MK-8718, GS-9500, GS-1156, and combinations thereof. For example, in some embodiments the additional therapeutic agent is selected from saquinavir, ritonavir, indinavir, nelfinavir, amprenavir, lopinavir, atazanavir, fosamprenavir, darunavir, tipranavir, cobicistat. In some embodiments, the additional therapeutic agent is selected from amprenavir, atazanavir, brecanavir, darunavir, fosamprenavir, fosamprenavir calcium, indinavir, indinavir sulfate, lopinavir, nelfinavir, nelfinavir mesylate, ritonavir, saquinavir, saquinavir mesylate, tipranavir, DG-17, TMB-657 (PPL-100), T-169, BL-008, MK-8122, TMB-607, TMC-310911, and combinations thereof.

In some embodiments, the additional therapeutic agent is a HIV integrase inhibitor. For example, in some embodiment, the additional therapeutic agent is selected from raltegravir, elvitegravir, dolutegravir, abacavir, lamivudine, bictegravir and combinations thereof. In some embodiment, the additional therapeutic agent is bictegravir. In some embodiments, the additional therapeutic agent is selected from a group consisting of bictegravir, elvitegravir, curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, raltegravir, dolutegravir, JTK-351, bictegravir, AVX-15567, BMS-986197, cabotegravir (long-acting injectable), diketo quinolin-4-1 derivatives, integrase-LEDGF inhibitor, ledgins, M-522, M-532, NSC-310217, NSC-371056, NSC-48240, NSC-642710, NSC-699171, NSC-699172, NSC-699173, NSC-699174, stilbenedisulfonic acid, T-169, VM-3500, cabotegravir, and combinations thereof.

In some embodiments, the additional therapeutic agent is a HIV entry inhibitor. For example, in some embodiments, the additional therapeutic agent is selected from enfuvirtide, maraviroc, and combinations thereof. Further examples of HIV entry inhibitors include, but are not limited to, cenicriviroc, CCR5 inhibitors, gp41 inhibitors, CD4 attachment inhibitors, DS-003 (BMS-599793), gp120 inhibitors, and CXCR4 inhibitors. Examples of CCR5 inhibitors include aplaviroc, vicriviroc, maraviroc, cenicriviroc, leronlimab (PRO-140), adaptavir (RAP-101), nifeviroc (TD-0232), anti-GP120/CD4 or CCR5 bispecific antibodies, B-07, MB-66, polypeptide C25P, TD-0680, and vMIP (Haimipu). Examples of CXCR4 inhibitors include plerixafor, ALT-1188, N15 peptide, and vMIP (Haimipu).

In some embodiments, the additional therapeutic agent is a HIV nucleoside reverse transcriptase inhibitors. In some embodiments, the additional therapeutic agent is a HIV nonnucleoside reverse transcriptase inhibitors. In some embodiments, the additional therapeutic agent is an acyclic nucleoside phosphonate analogue. In some embodiments, the additional therapeutic agent is a HIV capsid inhibitor.

In some embodiments, the additional therapeutic agent is a HIV nucleoside or nucleotide inhibitor of reverse transcriptase. For example, the additional therapeutic agent is selected from adefovir, adefovir dipivoxil, azvudine, emtricitabine, tenofovir, tenofovir alafenamide, tenofovir alafenamide fumarate, tenofovir alafenamide hemifumarate, tenofovir disoproxil, tenofovir disoproxil fumarate, tenofovir disoproxil hemifumarate, VIDEX® and VIDEX EC® (didanosine, ddl), abacavir, abacavir sulfate, alovudine, apricitabine, censavudine, didanosine, elvucitabine, festinavir, fosalvudine tidoxil, CMX-157, dapivirine, doravirine, etravirine, OCR-5753, tenofovir disoproxil orotate, fozivudine tidoxil, islatravir, lamivudine, phosphazid, stavudine, zalcitabine, zidovudine, rovafovir etalafenamide (GS-9131), GS-9148, MK-8504, MK-8591, MK-858, VM-2500, KP-1461, and combinations thereof.

In some embodiments, the additional therapeutic agent is a HIV non-nucleoside or non-nucleotide inhibitor of reverse transcriptase. For example, the additional agent is selected from dapivirine, delavirdine, delavirdine mesylate, doravirine, efavirenz, etravirine, lentinan, MK-8583, nevirapine, rilpivirine, TMC-278LA, ACC-007, AIC-292, KM-023, PC-1005, elsulfavirine rilp (VM-1500), combinations thereof.

In some embodiments, the additional therapeutic agents are selected from ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); adefovir; adefovir dipivoxil; cobicistat; emtricitabine; tenofovir; tenofovir disoproxil; tenofovir disoproxil fumarate; tenofovir alafenamide; tenofovir alafenamide hemifumarate; TRIUMEQ® (dolutegravir, abacavir, and lamivudine); dolutegravir, abacavir sulfate, and lamivudine; raltegravir; raltegravir and lamivudine; maraviroc; enfuvirtide; ALUVIA® (KALETRA®; lopinavir and ritonavir); COMBIVIR® (zidovudine and lamivudine; AZT+3TC); EPZICOM® (LIVEXA®; abacavir sulfate and lamivudine; ABC+3TC); TRIZIVIR® (abacavir sulfate, zidovudine, and lamivudine; ABC+AZT+3TC); rilpivirine; rilpivirine hydrochloride; atazanavir sulfate and cobicistat; atazanavir and cobicistat; darunavir and cobicistat; atazanavir; atazanavir sulfate; dolutegravir; elvitegravir; ritonavir; atazanavir sulfate and ritonavir; darunavir; lamivudine; prolastin; fosamprenavir; fosamprenavir calcium efavirenz; etravirine; nelfinavir; nelfinavir mesylate; interferon; didanosine; stavudine; indinavir; indinavir sulfate; tenofovir and lamivudine; zidovudine; nevirapine; saquinavir; saquinavir mesylate; aldesleukin; zalcitabine; tipranavir; amprenavir; delavirdine; delavirdine mesylate; Radha-108 (receptol); lamivudine and tenofovir disoproxil fumarate; efavirenz, lamivudine, and tenofovir disoproxil fumarate; phosphazid; lamivudine, nevirapine, and zidovudine; abacavir; and abacavir sulfate.

In some embodiments, the additional therapeutic agent is selected from colistin, valrubicin, icatibant, bepotastine, epirubicin, epoprosetnol, vapreotide, aprepitant, caspofungin, perphenazine, atazanavir, efavirenz, ritonavir, acyclovir, ganciclovir, penciclovir, prulifloxacin, bictegravir, nelfinavir, tegobuvi, nelfinavir, praziquantel, pitavastatin, perampanel, eszopiclone, and zopiclone.

In some embodiments, the additional therapeutic agent is an inhibitor of Bruton tyrosine kinase (BTK, AGMX1, AT, ATK, BPK, IGHD3, ID1, PSCTK1, XLA; NCBI Gene ID: 695). For example, in some embodiments, the additional therapeutic agent is selected from (S)-6-amino-9-(1-(but-2-ynoyl)pyrrolidin-3-yl)-7-(4-phenoxyphenyl)-7H-purin-8(9H)-one, acalabrutinib (ACP-196), BGB-3111, CB988, HM71224, ibrutinib (Imbruvica), M-2951 (evobrutinib), M7583, tirabrutinib (ONO-4059), PRN-1008, spebrutinib (CC-292), TAK-020, vecabrutinib, ARQ-531, SHR-1459, DTRMWXHS-12, TAS-5315, AZD6738, calquence, danvatirsen, and combinations thereof. In some embodiments, the additional therapeutic agent is selected from a group consisting of tirabrutinib, ibrutinib, acalabrutinib, and combinations thereof. In some embodiments, the additional therapeutic agent is selected from a group consisting of tirabrutinib, ibrutinib, and combinations thereof. In some embodiments, the additional therapeutic agent is tyrphostin A9 (A9).

In some embodiments, the additional therapeutic agent is a KRAS inhibitor. For example, in some embodiments, the additional therapeutic agent is selected from AMG-510, COTI-219, MRTX-1257, ARS-3248, ARS-853, WDB-178, BI-3406, BI-1701963, ARS-1620 (G12C), SML-8-73-1 (G12C), Compound 3144 (G12D), Kobe0065/2602 (Ras GTP), RT11, MRTX-849 (G12C) and K-Ras(G12D)-selective inhibitory peptides, including KRpep-2 (Ac-RRCPLYISYDPVCRR-NH2), KRpep-2d (Ac-RRRRCPLYISYDPVCRRRR-NH2), and combinations thereof.

In some embodiments, the additional therapeutic agent is a proteasome inhibitor. For example, in some embodiments, the additional therapeutic agent is selected from a group consisting of ixazomib, carfilzomib, marizomib, bortezomib, and combinations thereof. In some embodiments, the additional therapeutic agent is carfilzomib.

In some embodiments, the additional therapeutic agent is a vaccine. For example, in some embodiments, the additional therapeutic agent is a DNA vaccine, RNA vaccine, live-attenuated vaccine, therapeutic vaccine, prophylactic vaccine, protein based vaccine, or a combination thereof. In some embodiments, the additional therapeutic agent is mRNA-1273. In some embodiments, the additional therapeutic agent is INO-4800 or INO-4700. In some embodiments, the additional therapeutic agent is live-attenuated RSV vaccine MEDI-559, human monoclonal antibody REGN2222 against RSV, palivizumab, respiratory syncytial virus immune globulin, intravenous [RSV-IGIV], and combinations thereof. In some embodiments, the additional therapeutic agent is a HBV vaccine, for example pediarix, engerix-B, and recombivax HB. In some embodiments, the additional therapeutic agent is a VZV vaccine, for example zostavax and varivax. In some embodiments, the additional therapeutic agent is a HPV vaccine, for example cervarix, gardasil 9, and gardasil. In some embodiments, the additional therapeutic agent is an influenza virus vaccine. For example, a (i) monovalent vaccine for influenza A (e.g., influenza A [H5N1] virus monovalent vaccine and influenza A [H1N1] 2009 virus monovalent vaccines), (ii) trivalent vaccine for influenza A and B viruses (e.g., Afluria, Agriflu, Fluad, Fluarix, Flublok, Flucelvax, FluLaval, Fluvirin, and Fluzone), and (iii) quadrivalent vaccine for influenza A and B viruses (FluMist, Fluarix, Fluzone, and FluLaval). In some embodiments, the additional therapeutic agent is a human adenovirus vaccine (e.g., Adenovirus Type 4 and Type 7 Vaccine, Live, Oral). In some embodiments, the additional therapeutic agent is a rotavirus vaccine (e.g., Rotarix for rotavirus serotype G1, G3, G4, or G9 and RotaTeq for rotavirus serotype G1, G2, G3, or G4). In some embodiments, the additional therapeutic agent is a hepatitis A virus vaccine (e.g., Havrix and Vagta). In some embodiments, the additional therapeutic agent is poliovirus vaccines (e.g., Kinrix, Quadracel, and Ipol). In some embodiments, the additional therapeutic agent is a yellow fever virus vaccine (e.g., YF-Vax). In some embodiments, the additional therapeutic agent is a Japanese encephalitis virus vaccines (e.g., Ixiaro and JE-Vax). In some embodiments, the additional therapeutic agent is a measles vaccine (e.g., M-M-R II and ProQuad). In some embodiments, the additional therapeutic agent is a mumps vaccine (e.g., M-M-R II and ProQuad). In some embodiments, the additional therapeutic agent is a rubella vaccine (e.g., M-M-R II and ProQuad). In some embodiments, the additional therapeutic agent is a varicella vaccine (e.g., ProQuad). In some embodiments, the additional therapeutic agent is a rabies vaccine (e.g., Imovax and RabAvert). In some embodiments, the additional therapeutic agent is a variola virus (smallpox) vaccine (ACAM2000). In some embodiments, the additional therapeutic agent is a and hepatitis E virus (HEV) vaccine (e.g., HEV239). In some embodiments, the additional therapeutic agent is a 2019-nCov vaccine.

In some embodiments, the additional therapeutic agent is an antibody, for example a monoclonal antibody. For example, the additional therapeutic agent is an antibody against 2019-nCov selected from the Regeneron antibodies, the Wuxi Antibodies, the Vir Biotechnology Antibodies, antibodies that target the SARS-CoV-2 spike protein, antibodies that can neutralize SARS-CoV-2 (SARS-CoV-2 neutralizing antibodies), and combinations thereof. In some embodiments, the additional therapeutic agent is anti-SARS-CoV antibody CR-3022. In some embodiments, the additional therapeutic agent is aPD-1 antibody.

In some embodiments, the additional therapeutic agent is recombinant cytokine gene-derived protein injection.

In some embodiments, the additional therapeutic agent is a polymerase inhibitor. In some embodiments, the additional therapeutic agent is a DNA polymerase inhibitor. For example, in some embodiments, the additional therapeutic agent is cidofovir. In some embodiments, the additional therapeutic agent is a RNA polymerase inhibitor. For example, in some embodiments, the additional therapeutic agent is selected from ribavirin, favipiravir, lamivudine, pimodivir and combination thereof.

In some embodiments, the additional therapeutic agent is selected from lopinavir, ritonavir, interferon-alpha-2b, ritonavir, arbidol, hydroxychloroquine, darunavir and cobicistat, abidol hydrochloride, oseltamivir, litonavir, emtricitabine, tenofovir alafenamide fumarate, baloxavir marboxil, ruxolitinib, and combinations thereof.

In some embodiments, the additional therapeutic agent is selected from 6′-fluorinated aristeromycin analogues, acyclovir fleximer analogues, disulfiram, thiopurine analogues, ASC09F, GC376, GC813, phenylisoserine derivatives, neuroiminidase inhibitor analogues, pyrithiobac derivatives, bananins and 5-hydroxychromone derivatives, SSYA10-001, griffithsin, HR2P-M1, HR2P-M2, P21S10, Dihydrotanshinone E-64-C and E-64-D, OC43-HR2P, MERS-SHB, 229E-HR1P, 229E-HR2P, resveratrol, 1-thia-4-azaspiro[4.5] decan-3-one derivatives, gemcitabine hydrochloride, loperamide, recombinant interferons, cyclosporine A, alisporivir, imatinib mesylate, dasatinib, selumetinib, trametinib, rapamycin, saracatinib, chlorpromazine, triflupromazine, fluphenazine, thiethylperazine, promethazine, cyclophilin inhibitors, K11777, camostat, k22, teicoplanin derivatives, benzo-heterocyclic amine derivatives N30, mycophenolic acid, silvestrol, and combinations thereof.

In some embodiments, the additional therapeutic agent is an antibody. In some embodiments, the additional therapeutic agent is an antibody that binds to a coronavirus, for example an antibody that binds to SARS-CoV or MERS-CoV. In some embodiments, the additional therapeutic agent is a of 2019-nCoV virus antibody.

Compositions of the invention are also used in combination with other active ingredients. For the treatment of 2019-nCoV virus infections, preferably, the other active therapeutic agent is active against coronavirus infections, for example 2019-nCoV virus infections. The compounds and compositions of the present invention are also intended for use with general care provided patients with 2019-nCoV viral infections, including parenteral fluids (including dextrose saline and Ringer's lactate) and nutrition, antibiotic (including metronidazole and cephalosporin antibiotics, such as ceftriaxone and cefuroxime) and/or antifungal prophylaxis, fever and pain medication, antiemetic (such as metoclopramide) and/or antidiarrheal agents, vitamin and mineral supplements (including Vitamin K and zinc sulfate), anti-inflammatory agents (such as ibuprofen or steroids), corticosteroids such as methylprednisolone, immunomodulatory medications (e.g., interferon), other small molecule or biologics antiviral agents targeting 2019-nCoV (such as but not limited to lopinavir/ritonavir, EIDD-1931, favipiravir, ribavirine, neutralizing antibodies, etc.), vaccines, pain medications, and medications for other common diseases in the patient population, such anti-malarial agents (including artemether and artesunate-lumefantrine combination therapy), typhoid (including quinolone antibiotics, such as ciprofloxacin, macrolide antibiotics, such as azithromycin, cephalosporin antibiotics, such as ceftriaxone, or aminopenicillins, such as ampicillin), or shigellosis. In some embodiments, the additional therapeutic agent is dihydroartemisinin/piperaquine. In some embodiments, the additional therapeutic agent is EIDD-2801 (MH-4482, Molnupiravir).

In some embodiments, the additional therapeutic agent is an immunomodulator. Examples of immune-based therapies include toll-like receptors modulators such as tlr1, tlr2, tlr3, tlr4, tlr5, tlr6, tlr7, tlr8, tlr9, tlr10, tlr1 1, tlr12, and tlr13; programmed cell death protein 1 (Pd-1) modulators; programmed death-ligand 1 (Pd-L1) modulators; IL-15 modulators; DermaVir; interleukin-7; plaquenil (hydroxychloroquine); proleukin (aldesleukin, IL-2); interferon alfa; interferon alfa-2b; interferon alfa-n3; pegylated interferon alfa; interferon gamma; hydroxyurea; mycophenolate mofetil (MPA) and its ester derivative mycophenolate mofetil (MMF); ribavirin; polymer polyethyleneimine (PEI); gepon; IL-12; WF-10; VGV-1; MOR-22; BMS-936559; CYT-107, interleukin-15/Fc fusion protein, AM-0015, ALT-803, NIZ-985, NKTR-255, NKTR-262, NKTR-214, normferon, peginterferon alfa-2a, peginterferon alfa-2b, recombinant interleukin-15, Xmab-24306, RPI-MN, STING modulators, RIG-I modulators, NOD2 modulators, SB-9200, and IR-103. In some embodiments, the additional therapeutic agent is fingolimod, leflunomide, or a combination thereof. In some embodiments, the additional therapeutic agent is thalidomide.

In some embodiments, the additional therapeutic agent is an IL-6 inhibitor, for example tocilizumab, sarilumab, or a combination thereof.

In some embodiments, the additional therapeutic agent is an anti-TNF inhibitor. For example, the additional therapeutic agent is adalimumab, etanercept, golimumab, infliximab, or a combination thereof.

In some embodiments, the additional therapeutic agent is a JAK inhibitor, for example the additional therapeutic agent is baricitinib, filgotinib, olumiant, or a combination thereof.

In some embodiments, the additional therapeutic agent is an inflammation inhibitor, for example pirfenidone.

In some embodiments, the additional therapeutic agent is an antibiotic for secondary bacterial pneumonia. For example, the additional therapeutic agent is macrolide antibiotics (e.g., azithromycin, clarithromycin, and mycoplasmapneumoniae), fluoroquinolones (e.g., ciprofloxacin and levofloxacin), tetracyclines (e.g., doxycycline and tetracycline), or a combination thereof.

In some embodiments, the compounds described herein are used in combination with pneumonia standard of care (see e.g., Pediatric Community Pneumonia Guidelines, CID 2011:53 (1 October)). Treatment for pneumonia generally involves curing the infection and preventing complications. Specific treatment will depend on several factors, including the type and severity of pneumonia, age and overall health of the individuals. The options include: (i) antibiotics, (ii) cough medicine, and (iii) fever reducers/pain relievers (for e.g., aspirin, ibuprofen (Advil, Motrin IB, others) and acetaminophen (Tylenol, others)). In some embodiments, the additional therapeutic agent is bromhexine anti-cough.

In some embodiments, the compounds described herein are used in combination with immunoglobulin from cured COVID-19 patients. In some embodiments, the compounds described herein are used in combination with plasma transfusion. In some embodiments, the compounds described herein are used in combination with stem cells.

In some embodiments, the additional therapeutic agent is an TLR agonist. Examples of TLR agonists include, but are not limited to, vesatolimod (GS-9620), GS-986, IR-103, lefitolimod, tilsotolimod, rintatolimod, DSP-0509, AL-034, G-100, cobitolimod, AST-008, motolimod, GSK-1795091, GSK-2245035, VTX-1463, GS-9688, LHC-165, BDB-001, RG-7854, telratolimod, and RO-7020531.

In some embodiments, the additional therapeutic agent is selected from bortezomid, flurazepam, ponatinib, sorafenib, paramethasone, clocortolone, flucloxacillin, sertindole, clevidipine, atorvastatin, cinolazepam, clofazimine, fosaprepitant, and combinations thereof.

In some embodiments, the additional therapeutic agent is carrimycin, suramin, triazavirin, dipyridamole, bevacizumab, meplazumab, GD31 (rhizobium), NLRP inflammasome inhibitor, or α-ketoamine. In some embodiments, the additional therapeutic agent is recombinant human angiotensin-converting enzyme 2 (rhACE2). In some embodiments, the additional therapeutic agent is viral macrophage inflammatory protein (vMIP).

In some embodiments, the additional therapeutic agent is an anti-viroporin therapeutic. For example, the additional therapeutic agent is BIT-314 or BIT-225. In some embodiments, the additional therapeutic agent is coronavirus E protein inhibitor. For example, the additional therapeutic agent is BIT-009. Further examples of additional therapeutic agents include those described in WO-2004112687, WO-2006135978, WO-2018145148, and WO-2009018609.

In some embodiments, the additional therapeutic or prophylactic agent is molnupiravir, oseltamivir, nirmatrelvir, or ritonavir. In some embodiments, the additional therapeutic or prophylactic agent is ritonavir or cobicistat.

In some embodiments, the additional therapeutic agent a 2,5-Oligoadenylate synthetase stimulator, 5-HT 2a receptor antagonist, 5-Lipoxygenase inhibitor, ABL family tyrosine kinase inhibitor, Abl tyrosine kinase inhibitor, Acetaldehyde dehydrogenase inhibitor, Acetyl CoA carboxylase inhibitor, Actin antagonist, Actin modulator, Activity-dependent neuroprotector modulator, Adenosine A3 receptor agonist, Adrenergic receptor antagonist, Adrenomedullin ligand, Adrenomedullin ligand inhibitor, Advanced glycosylation product receptor antagonist, Advanced glycosylation product receptor modulator, AKT protein kinase inhibitor, Alanine proline rich secreted protein stimulator, Aldose reductase inhibitor, Alkaline phosphatase stimulator, Alpha 2 adrenoceptor antagonist, Alpha 2B adrenoceptor agonist, AMP activated protein kinase stimulator, AMPA receptor modulator, Amyloid protein deposition inhibitor, Androgen receptor antagonist, Angiotensin II AT-1 receptor antagonist, Angiotensin II AT-2 receptor agonist, Angiotensin II receptor modulator, Angiotensin converting enzyme 2 inhibitor, Angiotensin converting enzyme 2 modulator, Angiotensin converting enzyme 2 stimulator, Angiotensin receptor modulator, Annexin A5 stimulator, Anoctamin 1 inhibitor, Anti-coagulant, Anti-histamine, Anti-hypoxic, Anti-thrombotic, AP1 transcription factor modulator, Apelin receptor agonist, APOA1 gene stimulator, Apolipoprotein A1 agonist, Apolipoprotein B antagonist, Apolipoprotein B modulator, Apolipoprotein C3 antagonist, Aryl hydrocarbon receptor agonist, Aryl hydrocarbon receptor antagonist, ATP binding cassette transporter B5 modulator, Axl tyrosine kinase receptor inhibitor, Bactericidal permeability protein inhibitor, Basigin inhibitor, Basigin modulator, BCL2 gene inhibitor, BCL2L11 gene stimulator, Bcr protein inhibitor, Beta 1 adrenoceptor modulator, Beta 2 adrenoceptor agonist, Beta adrenoceptor agonist, Beta-arrestin stimulator, Blood clotting modulator, BMP10 gene inhibitor, BMP15 gene inhibitor, Bone morphogenetic protein-10 ligand inhibitor, Bone morphogenetic protein-15 ligand inhibitor, Bradykinin B2 receptor antagonist, Brain derived neurotrophic factor ligand, Bromodomain containing protein 2 inhibitor, Bromodomain containing protein 4 inhibitor, Btk tyrosine kinase inhibitor, C-reactive protein modulator, Ca2+ release activated Ca2+ channel 1 inhibitor, Cadherin-5 modulator, Calcium activated chloride channel inhibitor, Calcium channel modulator, Calpain-I inhibitor, Calpain-II inhibitor, Calpain-IX inhibitor, Cannabinoid CB2 receptor agonist, Cannabinoid receptor modulator, Casein kinase II inhibitor, CASP8-FADD-like regulator inhibitor, Caspase inhibitor, Catalase stimulator, CCL26 gene inhibitor, CCR2 chemokine antagonist, CCR5 chemokine antagonist, CD11a agonist, CD122 agonist, CD3 antagonist, CD4 agonist, CD40 ligand, CD40 ligand modulator, CD40 ligand receptor agonist, CD40 ligand receptor modulator, CD49d agonist, CD70 antigen modulator, CD73 agonist, CD73 antagonist, CD95 antagonist, CFTR inhibitor, CGRP receptor antagonist, Chemokine receptor-like 1 agonist, Chloride channel inhibitor, Chloride channel modulator, Cholera enterotoxin subunit B inhibitor, Cholesterol ester transfer protein inhibitor, Collagen modulator, Complement Cis subcomponent inhibitor, Complement C3 inhibitor, Complement C5 factor inhibitor, Complement C5a factor inhibitor, Complement Factor H stimulator, Complement cascade inhibitor, Complement factor C2 inhibitor, Complement factor D inhibitor, Connective tissue growth factor ligand inhibitor, Coronavirus nucleoprotein modulator, Coronavirus small envelope protein modulator, Coronavirus spike glycoprotein inhibitor, Coronavirus spike glycoprotein modulator, COVID19 envelope small membrane protein modulator, COVID19 non structural protein 8 modulator, COVID19 nucleoprotein modulator, COVID19 Protein 3a inhibitor, COVID19 replicase polyprotein 1a inhibitor, COVID19 replicase polyprotein 1a modulator, COVID19 replicase polyprotein 1ab inhibitor, COVID19 replicase polyprotein 1ab modulator, COVID19 Spike glycoprotein inhibitor, COVID19 Spike glycoprotein modulator, COVID19 structural glycoprotein modulator, CRF-2 receptor agonist, CSF-1 agonist, CSF-1 antagonist, CX3CR1 chemokine antagonist, CXC10 chemokine ligand inhibitor, CXC5 chemokine ligand inhibitor, CXCL1 gene modulator, CXCL2 gene modulator, CXCL3 gene modulator, CXCR1 chemokine antagonist, CXCR2 chemokine antagonist, CXCR4 chemokine antagonist, Cyclin D1 inhibitor, Cyclin E inhibitor, Cyclin-dependent kinase-1 inhibitor, Cyclin-dependent kinase-2 inhibitor, Cyclin-dependent kinase-5 inhibitor, Cyclin-dependent kinase-7 inhibitor, Cyclin-dependent kinase-9 inhibitor, Cyclooxygenase 2 inhibitor, Cyclooxygenase inhibitor, Cyclophilin inhibitor, Cysteine protease inhibitor, Cytochrome P450 3A4 inhibitor, Cytokine receptor antagonist, Cytotoxic T lymphocyte protein gene modulator, Cytotoxic T-lymphocyte protein-4 inhibitor, Cytotoxic T-lymphocyte protein-4 stimulator, DDX3 inhibitor, Dehydrogenase inhibitor, Dehydropeptidase-1 modulator, Deoxyribonuclease I stimulator, Deoxyribonuclease gamma stimulator, Deoxyribonuclease stimulator, Dihydroceramide delta 4 desaturase inhibitor, Dihydroorotate dehydrogenase inhibitor, Dipeptidyl peptidase I inhibitor, Dipeptidyl peptidase III inhibitor, Diuretic, DNA binding protein inhibitor, DNA methyltransferase inhibitor, Dopamine transporter inhibitor, E selectin antagonist, Ecto NOX disulfide thiol exchanger 2 inhibitor, EGFR gene inhibitor, Elongation factor 1 alpha 2 modulator, Endoplasmin modulator, Endoribonuclease DICER modulator, Endothelin ET-A receptor antagonist, Epidermal growth factor receptor antagonist, E-selectin antagonist, Estrogen receptor beta agonist, Estrogen receptor modulator, Eukaryotic initiation factor 4A-I inhibitor, Exo-alpha sialidase modulator, Exportin 1 inhibitor, Factor Ia modulator, Factor IIa modulator, Factor VII antagonist, Factor Xa antagonist, Factor XIa antagonist, FGF receptor antagonist, FGF-1 ligand, FGF-1 ligand inhibitor, FGF-2 ligand inhibitor, FGF1 receptor antagonist, FGF2 receptor antagonist, FGF3 receptor antagonist, Flt3 tyrosine kinase inhibitor, Fractalkine ligand inhibitor, Free fatty acid receptor 2 agonist, Free fatty acid receptor 3 agonist, furin inhibitors, Fyn tyrosine kinase inhibitor, FYVE finger phosphoinositide kinase inhibitor, G-protein coupled bile acid receptor 1 agonist, GABA A receptor modulator, Galectin-3 inhibitor, Gamma-secretase inhibitor, GDF agonist, Gelsolin stimulator, Glial cell neurotrophic factor ligand, Glucocorticoid receptor agonist, Glutathione peroxidase stimulator, GM-CSF ligand inhibitor, GM-CSF receptor agonist, GM-CSF receptor modulator, Griffithsin modulator, Growth regulated protein alpha ligand inhibitor, Grp78 calcium binding protein inhibitor, Heat shock protein HSP90 alpha inhibitor, Heat shock protein HSP90 beta inhibitor, Heat shock protein inhibitor, Heat shock protein stimulator, Hemagglutinin modulator, Hemoglobin modulator, Hemolysin alpha inhibitor, Heparanase inhibitor, Heparin agonist, Hepatitis B structural protein inhibitor, Hepatitis C virus NS5B polymerase inhibitor, HIF prolyl hydroxylase inhibitor, HIF prolyl hydroxylase-2 inhibitor, High mobility group protein B1 inhibitor, Histamine H1 receptor antagonist, Histamine H2 receptor antagonist, Histone deacetylase-6 inhibitor, Histone inhibitor, HIV protease inhibitor, HIV-1 gp120 protein inhibitor, HIV-1 protease inhibitor, HIV-1 reverse transcriptase inhibitor, HLA class I antigen modulator, HLA class II antigen modulator, Host cell factor modulator, Hsp 90 inhibitor, Human papillomavirus E6 protein modulator, Human papillomavirus E7 protein modulator, Hypoxia inducible factor inhibitor gene inhibitor, Hypoxia inducible factor-2 alpha modulator, I-kappa B kinase inhibitor, I-kappa B kinase modulator, ICAM-1 stimulator, IgG receptor FcRn large subunit p51 modulator, IL-12 receptor antagonist, IL-15 receptor agonist, IL-15 receptor modulator, IL-17 antagonist, IL-18 receptor accessory protein antagonist, IL-2 receptor agonist, IL-22 agonist, IL-23 antagonist, IL-6 receptor agonist, IL-6 receptor antagonist, IL-6 receptor modulator, IL-7 receptor agonist, IL-8 receptor antagonist, IL12 gene stimulator, IL8 gene modulator, Immunoglobulin G modulator, Immunoglobulin G1 agonist, Immunoglobulin G1 modulator, Immunoglobulin agonist, Immunoglobulin gamma Fc receptor I modulator, Immunoglobulin kappa modulator, Inosine monophosphate dehydrogenase inhibitor, Insulin sensitizer, Integrin agonist, Integrin alpha-4/beta-7 antagonist, Integrin alpha-V/beta-1 antagonist, Integrin alpha-V/beta-6 antagonist, Interferon agonist, Interferon alpha 14 ligand, Interferon alpha 2 ligand, Interferon alpha 2 ligand modulator, Interferon alpha ligand, Interferon alpha ligand inhibitor, Interferon alpha ligand modulator, Interferon beta ligand, Interferon gamma ligand inhibitor, Interferon gamma receptor agonist, Interferon gamma receptor antagonist, Interferon receptor modulator, Interferon type I receptor agonist, Interleukin 17A ligand inhibitor, Interleukin 17F ligand inhibitor, Interleukin 18 ligand inhibitor, Interleukin 22 ligand, Interleukin-1 beta ligand inhibitor, Interleukin-1 beta ligand modulator, Interleukin-1 ligand inhibitor, Interleukin-2 ligand, Interleukin-29 ligand, Interleukin-6 ligand inhibitor, Interleukin-7 ligand, Interleukin-8 ligand inhibitor, IRAK-4 protein kinase inhibitor, JAK tyrosine kinase inhibitor, Jak1 tyrosine kinase inhibitor, Jak2 tyrosine kinase inhibitor, Jak3 tyrosine kinase inhibitor, Jun N terminal kinase inhibitor, Jun N terminal kinase modulator, Kallikrein modulator, Kelch like ECH associated protein 1 modulator, Kit tyrosine kinase inhibitor, KLKB1 gene inhibitor, Lactoferrin stimulator, Lanosterol-14 demethylase inhibitor, Lck tyrosine kinase inhibitor, Leukocyte Ig like receptor A4 modulator, Leukocyte elastase inhibitor, Leukotriene BLT receptor antagonist, Leukotriene D4 antagonist, Leukotriene receptor antagonist, Listeriolysin stimulator, Liver X receptor antagonist, Low molecular weight heparin, Lung surfactant associated protein B stimulator, Lung surfactant associated protein D modulator, Lyn tyrosine kinase inhibitor, Lyn tyrosine kinase stimulator, Lysine specific histone demethylase 1 inhibitor, Macrophage migration inhibitory factor inhibitor, Mannan-binding lectin serine protease inhibitor, Mannan-binding lectin serine protease-2 inhibitor, MAO B inhibitor, MAP kinase inhibitor, MAPK gene modulator, Matrix metalloprotease modulator, Maxi K potassium channel inhibitor, MCL1 gene inhibitor, MEK protein kinase inhibitor, MEK-1 protein kinase inhibitor, Melanocortin MC1 receptor agonist, Melanocortin MC3 receptor agonist, Metalloprotease-12 inhibitor, METTL3 gene inhibitor, Moesin inhibitor, Moesin modulator, Monocyte chemotactic protein 1 ligand inhibitor, Monocyte differentiation antigen CD14 inhibitor, mRNA cap guanine N7 methyltransferase modulator, mTOR complex 1 inhibitor, mTOR complex 2 inhibitor, mTOR inhibitor, Mucolipin modulator, Muscarinic receptor antagonist, Myeloperoxidase inhibitor, NACHT LRR PYD domain protein 3 inhibitor, NAD synthase modulator, NADPH oxidase inhibitor, Neuropilin 2 modulator, Neuroplastin inhibitor, NFE2L2 gene stimulator, NK cell receptor agonist, NK1 receptor antagonist, NMDA receptor antagonist, NMDA receptor epsilon 2 subunit inhibitor, Non receptor tyrosine kinase TYK2 antagonist, Non-nucleoside reverse transcriptase inhibitor, nsp12 polymerase inhibitor, Nuclear erythroid 2-related factor 2 stimulator, Nuclear factor kappa B inhibitor, Nuclear factor kappa B modulator, Nuclease stimulator, Nucleolin inhibitor, Nucleoprotein inhibitor, Nucleoprotein modulator, Nucleoside reverse transcriptase inhibitor, Opioid receptor agonist, Opioid receptor antagonist, Opioid receptor mu modulator, Opioid receptor sigma antagonist 1, ORFlab polyprotein inhibitor, Ornithine decarboxylase inhibitor, Outer membrane protein inhibitor, OX40 ligand, p38 MAP kinase alpha inhibitor, p38 MAP kinase inhibitor, p38 MAP kinase modulator, p53 tumor suppressor protein stimulator, Palmitoyl protein thioesterase 1 inhibitor, Papain inhibitor, PARP inhibitor, PARP modulator, PDE 10 inhibitor, PDE 3 inhibitor, PDE 4 inhibitor, PDGF receptor alpha antagonist, PDGF receptor antagonist, PDGF receptor beta antagonist, Peptidyl-prolyl cis-trans isomerase A inhibitor, Peroxiredoxin 6 modulator, PGD2 antagonist, PGI2 agonist, P-glycoprotein inhibitor, Phosphoinositide 3-kinase inhibitor, Phosphoinositide-3 kinase delta inhibitor, Phosphoinositide-3 kinase gamma inhibitor, Phospholipase A2 inhibitor, PIKfyve inhibitor, Plasma kallikrein inhibitor, Plasminogen activator inhibitor 1 inhibitor, Platelet inhibitor, Platelet glycoprotein VI inhibitor, Polo-like kinase 1 inhibitor, Poly ADP ribose polymerase 1 inhibitor, Poly ADP ribose polymerase 2 inhibitor, Polymerase cofactor VP35 inhibitor, PPAR alpha agonist, Progesterone receptor agonist, Programmed cell death protein 1 modulator, Prolyl hydroxylase inhibitor, Prostaglandin E synthase-1 inhibitor, Protease inhibitor, Proteasome inhibitor, Protein arginine deiminase IV inhibitor, Protein tyrosine kinase inhibitor, Protein tyrosine phosphatase beta inhibitor, Protein tyrosine phosphatase-2C inhibitor, Proto-oncogene Mas agonist, Purinoceptor antagonist, Raf protein kinase inhibitor, RANTES ligand, Ras gene inhibitor, Retinoate receptor responder protein 2 stimulator, Rev protein modulator, Ribonuclease stimulator, RIP-1 kinase inhibitor, RNA helicase inhibitor, RNA polymerase inhibitor, RNA polymerase modulator, S phase kinase associated protein 2 inhibitor, SARS coronavirus 3C protease like inhibitor, Serine protease inhibitor, Serine threonine protein kinase ATR inhibitor, Serine threonine protein kinase TBK1 inhibitor, Serum amyloid A protein modulator, Signal transducer CD24 stimulator, Sirtuin inhibitor, Sodium channel stimulator, Sodium glucose transporter-2 inhibitor, Sphingosine kinase 1 inhibitor, Sphingosine kinase 2 inhibitor, Sphingosine kinase inhibitor, Sphingosine-1-phosphate receptor-1 agonist, Sphingosine-1-phosphate receptor-1 antagonist, Sphingosine-1-phosphate receptor-1 modulator, Sphingosine-1-phosphate receptor-5 agonist, Sphingosine-1-phosphate receptor-5 modulator, Spike glycoprotein inhibitor, Src tyrosine kinase inhibitor, STAT-1 modulator, STAT-3 inhibitor, STAT-5 inhibitor, STAT3 gene inhibitor, Stem cell antigen-1 inhibitor, Stimulator of interferon genes protein stimulator, Sulfatase inhibitor, Superoxide dismutase modulator, Superoxide dismutase stimulator, Syk tyrosine kinase inhibitor, T cell immunoreceptor Ig ITIM protein inhibitor, T cell receptor agonist, T cell surface glycoprotein CD28 inhibitor, T-cell differentiation antigen CD6 inhibitor, T-cell surface glycoprotein CD8 stimulator, T-cell transcription factor NFAT modulator, Tankyrase-1 inhibitor, Tankyrase-2 inhibitor, Tek tyrosine kinase receptor stimulator, Telomerase modulator, Tetanus toxin modulator, TGF beta receptor antagonist, TGFB2 gene inhibitor, Thymosin beta 4 ligand, Thyroid hormone receptor beta agonist, Tissue factor inhibitor, Tissue plasminogen activator modulator, Tissue plasminogen activator stimulator, TLR agonist, TLR modulator, TLR-2 agonist, TLR-2 antagonist, TLR-3 agonist, TLR-4 agonist, TLR-4 antagonist, TLR-6 agonist, TLR-7 agonist, TLR-7 antagonist, TLR-8 antagonist, TLR-9 agonist, TMPRSS2 gene inhibitor, TNF alpha ligand inhibitor, TNF alpha ligand modulator, TNF binding agent, TNF gene inhibitor, Topoisomerase inhibitor, Transcription factor EB stimulator, Transferrin modulator, Transketolase inhibitor, Translocation associated protein inhibitor, Transmembrane serine protease 2 inhibitor, Transthyretin modulator, TREM receptor 1 antagonist, TRP cation channel C1 modulator, TRP cation channel C6 inhibitor, TRP cation channel V6 inhibitor, Trypsin 1 inhibitor, Trypsin 2 inhibitor, Trypsin 3 inhibitor, Trypsin inhibitor, Tubulin alpha inhibitor, Tubulin beta inhibitor, Tumor necrosis factor 14 ligand inhibitor, TYK2 gene inhibitor, Type I IL-1 receptor antagonist, Tyrosine protein kinase ABL1 inhibitor, Ubiquinol cytochrome C reductase 14 kDa inhibitor, Ubiquitin ligase modulator, Unspecified GPCR agonist, Unspecified cytokine receptor modulator, Unspecified enzyme stimulator, Unspecified gene inhibitor, Unspecified receptor modulator, Urokinase plasminogen activator inhibitor, Vascular cell adhesion protein 1 agonist, Vasodilator, VEGF ligand inhibitor, VEGF receptor antagonist, VEGF-1 receptor antagonist, VEGF-1 receptor modulator, VEGF-2 receptor antagonist, VEGF-3 receptor antagonist, Vimentin inhibitor, Vimentin modulator, VIP receptor agonist, Viral envelope protein inhibitor, Viral protease inhibitor, Viral protease modulator, Viral protein target modulator, Viral ribonuclease inhibitor, Viral structural protein modulator, Vitamin D3 receptor agonist, X-linked inhibitor of apoptosis protein inhibitor, Xanthine oxidase inhibitor, or Zonulin inhibitor.

In some embodiments, the compounds and compositions of the present disclosure may be administered in combination with a Sars-Cov-2 treatment, such as parenteral fluids (including dextrose saline and Ringer's lactate), nutrition, antibiotics (including azithromycin, metronidazole, amphotericin B, amoxicillin/clavulanate, trimethoprim/sulfamethoxazole, R-327 and cephalosporin antibiotics, such as ceftriaxone and cefuroxime), antifungal prophylaxis, fever and pain medication, antiemetic (such as metoclopramide) and/or antidiarrheal agents, vitamin and mineral supplements (including Vitamin K, vitamin D, cholecalciferol, vitamin C and zinc sulfate), anti-inflammatory agents (such as ibuprofen or steroids), corticosteroids such as dexamethasone, methylprednisolone, prednisone, mometasone, immunomodulatory medications (eg interferon), vaccines, and pain medications.

In some embodiments, the additional therapeutic agent is an Abl tyrosine kinase inhibitor, such as radotinib or imatinib.

In some embodiments, the additional therapeutic agent is an acetaldehyde dehydrogenase inhibitor, such as ADX-629.

In some embodiments, the additional therapeutic agent is an adenosine A3 receptor agonist, such as piclidenoson.

In some embodiments, the additional therapeutic agent is an adrenomedullin ligand such as adrenomedullin.

In some embodiments, the additional therapeutic agent is a p38 MAPK+PPAR gamma agonist/insulin sensitizer such as KIN-001.

In some embodiments, the additional therapeutic agent is a PPAR alpha agonist such as DWTC-5101 (fenofibrate choline).

In some embodiments, the additional therapeutic agent is a cyclophilin inhibitor such as rencofilstat.

In some embodiments, the additional therapeutic is a p38 MAP kinase inhibitor such as PRX-201 or Gen-1124.

In some embodiments, the additional therapeutic agent is an aldose reductase inhibitor, such as caficrestat.

In some embodiments, the additional therapeutic agent is an AMPA receptor modulator, such as traneurocin.

In some embodiments, the additional therapeutic agent is an annexin A5 stimulator, such as AP-01 or SY-005.

In some embodiments, the additional therapeutic agent is an apelin receptor agonist, such as CB-5064MM.

In some embodiments, the additional therapeutic agent is an anti-coagulant, such as heparins (heparin and low molecular weight heparin), aspirin, apixaban, dabigatran, edoxaban, argatroban, enoxaparin, or fondaparinux.

In some embodiments, the additional therapeutic agent is an androgen receptor antagonist such as bicalutamide, deutenzalutamide, enzalutamide, or pruxelutamide (proxalutamide).

In some embodiments, the additional therapeutic agent is anti-hypoxic, such as trans-sodium crocetinate.

In some embodiments, the additional therapeutic agent is an anti-thrombotic, such as defibrotide, rivaroxaban, alteplase, tirofiban, clopidogrel, prasugrel, bemiparin, bivalirudin, sulodexide, or tenecteplase.

In some embodiments, the additional therapeutic agent is an antihistamine, such as cloroperastine or clemastine.

In some embodiments, the additional therapeutic agent is an apolipoprotein A1 agonist, such as CER-001.

In some embodiments, the additional therapeutic agent is a phospholipase A2 inhibitor, such as icosapent ethyl.

In some embodiments, the additional therapeutic agent is an axl tyrosine kinase receptor inhibitor, such as bemcentinib.

In some embodiments, the additional therapeutic agent is a corticosteroid/beta 2 adrenoceptor agonist, such as budesonide+formoterol fumarate.

In some embodiments, the additional therapeutic agent is a BET bromodomain inhibitor/APOA1 gene stimulator such as apabetalone.

In some embodiments, the additional therapeutic agent is a blood clotting modulator, such as lanadelumab.

In some embodiments, the additional therapeutic agent is a bradykinin B2 receptor antagonist, such as icatibant.

In some embodiments, the additional therapeutic agent is an EGFR gene inhibitor/Btk tyrosine kinase inhibitor, such as abivertinib.

In some embodiments, the additional therapeutic agent is a Btk tyrosine kinase inhibitor, such as ibrutinib or zanubrutinib.

In some embodiments, the additional therapeutic agent is a calpain-III/IX inhibitor, such as BLD-2660.

In some embodiments, the additional therapeutic agent is a cannabinoid CB2 receptor agonist, such as onternabez or PPP-003.

In some embodiments, the additional therapeutic agent is a Ca2+ release activated Ca2+ channel 1 inhibitor, such as zegocractin (CM-4620).

In some embodiments, the additional therapeutic agent is an ATR inhibitor, such as berzosertib.

In some embodiments, the additional therapeutic agent is a cadherin-5 modulator, such as FX-06.

In some embodiments, the additional therapeutic agent is a casein kinase II inhibitor, such as silmitasertib.

In some embodiments, the additional therapeutic agent is a caspase inhibitor, such as emricasan.

In some embodiments, the additional therapeutic agent is a catalase stimulator/superoxide dismutase stimulator, such as MP-1032.

In some embodiments, the additional therapeutic agent is a CCR2 chemokine antagonist/CCR5 chemokine antagonist such as cenicriviroc.

In some embodiments, the additional therapeutic agent is a CCR5 chemokine antagonist, such as maraviroc or leronlimab.

In some embodiments, the additional therapeutic agent is a CD122 agonist/IL-2 receptor agonist, such as bempegaldesleukin.

In some embodiments, the additional therapeutic agent is a CD73 agonist/interferon beta ligand, such as FP-1201.

In some embodiments, the additional therapeutic agent is a cholesterol ester transfer protein inhibitor, such as dalcetrapib.

In some embodiments, the additional therapeutic agent is a Mannan-binding lectin serine protease/complement Cls subcomponent inhibitor/myeloperoxidase inhibitor, such as RLS-0071.

In some embodiments, the additional therapeutic agent is a complement C₅ factor inhibitor/leukotriene BLT receptor antagonist, such as nomacopan.

In some embodiments, the additional therapeutic agent is a complement C₅ factor inhibitor, such as eculizumab, STSA-1002, zilucoplan.

In some embodiments, the additional therapeutic agent is a CXCR4 chemokine antagonist, such as plerixafor or motixafortide.

In some embodiments, the additional therapeutic agent is a cytochrome P450 3A4 inhibitor/peptidyl-prolyl cis-trans isomerase A inhibitor, such as alisporivir.

In some embodiments, the additional therapeutic agent is a cysteine protease inhibitor, such as SLV-213.

In some embodiments, the additional therapeutic agent is a dihydroorotate dehydrogenase inhibitor, such as Meds-433, brequinar, RP-7214, farudostat or emvododstat.

In some embodiments, the additional therapeutic agent is a dehydropeptidase-1 modulator, such as Metablok.

In some embodiments, the additional therapeutic agent is a dihydroorotate dehydrogenase inhibitor/IL-17 antagonist, such as vidofludimus.

In some embodiments, the additional therapeutic agent is a diuretic, such as an aldosterone antagonist, such as spironolactone.

In some embodiments, the additional therapeutic agent is a deoxyribonuclease I stimulator, such as GNR-039 or dornase alfa.

In some embodiments, the additional therapeutic agent is a NET inhibitor, such as NTR-441.

In some embodiments, the additional therapeutic agent is a dihydroceramide delta 4 desaturase inhibitor/sphingosine kinase 2 inhibitor, such as opaganib.

In some embodiments, the additional therapeutic agent is a DNA methyltransferase inhibitor, such as azacytidine.

In some embodiments, the additional therapeutic agent is an LXR antagonist, such as larsucosterol.

In some embodiments, the additional therapeutic agent is a dipeptidyl peptidase I inhibitor, such as brensocatib.

In some embodiments, the additional therapeutic agent is a protein arginine deiminase IV inhibitor, such as JBI-1044.

In some embodiments, the additional therapeutic agent is an elongation factor 1 alpha 2 modulator, such as plitidepsin.

In some embodiments, the additional therapeutic agent is a eukaryotic initiation factor 4A-I inhibitor, such as zotatifin.

In some embodiments, the additional therapeutic agent is an exo-alpha sialidase modulator, such as DAS-181.

In some embodiments, the additional therapeutic agent is an exportin 1 inhibitor, such as selinexor.

In some embodiments, the additional therapeutic agent is a fractalkine ligand inhibitor, such as KAND-567.

In some embodiments, the additional therapeutic agent is a FYVE finger phosphoinositide kinase inhibitor/IL-12 receptor antagonist/IL-23 antagonist, such as apilimod dimesylate.

In some embodiments, the additional therapeutic agent is a GABA A receptor modulator, such as brexanolone.

In some embodiments, the additional therapeutic agent is a glucocorticoid receptor agonist, such as ciclesonide, hydrocortisone, dexamethasone, dexamethasone phosphate, or 101-PGC-005.

In some embodiments, the additional therapeutic agent is a GM-CSF receptor agonist, such as sargramostim.

In some embodiments, the additional therapeutic agent is a GPCR agonist, such as esuberaprost sodium.

In some embodiments, the additional therapeutic agent is a Griffithsin modulator, such as Q-Griffithsin.

In some embodiments, the additional therapeutic agent is a leukotriene D4 antagonist, such as montelukast.

In some embodiments, the additional therapeutic agent is a histamine H1 receptor antagonist, such as ebastine, tranilast, levocetirizine dihydrochloride.

In some embodiments, the additional therapeutic agent is a histamine H2 receptor antagonist, such as famotidine.

In some embodiments, the additional therapeutic agent is a heat shock protein stimulator/insulin sensitizer/PARP inhibitor, such as BGP-15.

In some embodiments, the additional therapeutic agent is a histone inhibitor, such as STC-3141.

In some embodiments, the additional therapeutic agent is a histone deacetylase-6 inhibitor, such as CKD-506.

In some embodiments, the additional therapeutic agent is a HIF prolyl hydroxylase-2 inhibitor, such as desidustat.

In some embodiments, the additional therapeutic agent is an HIF prolyl hydroxylase inhibitor, such as vadadustat.

In some embodiments, the additional therapeutic agent is an IL-8 receptor antagonist, such as reparixin.

In some embodiments, the additional therapeutic agent is an IL-7 receptor agonist, such as CYT-107.

In some embodiments, the additional therapeutic agent is an IL-7 receptor agonist/interleukin-7 ligand, such as efineptakin alfa.

In some embodiments, the additional therapeutic agent is an IL-22 agonist, such as efmarodocokin alfa.

In some embodiments, the additional therapeutic agent is an IL-22 agonist/interleukin 22 ligand, such as F-652.

In some embodiments, the additional therapeutic agent is targeted to IL-33, such as tozorakimab.

In some embodiments, the additional therapeutic is an IL-15 agonist such as nogapendekin alfa.

In some embodiments, the additional therapeutic agent is an integrin alpha-V/beta-1 antagonist/integrin alpha-V/beta-6 antagonist, such as bexotegrast.

In some embodiments, the additional therapeutic agent is an interferon alpha 2 ligand, such as interferon alfa-2b or Virafin.

In some embodiments, the additional therapeutic agent is an interferon beta ligand, such as interferon beta-1a follow-on biologic, interferon beta-1b, or SNG-001.

In some embodiments, the additional therapeutic agent is an interferon receptor modulator, such as peginterferon lambda-1a.

In some embodiments, the additional therapeutic agent is an interleukin-2 ligand, such as aldesleukin.

In some embodiments, the additional therapeutic agent is an IRAK-4 protein kinase inhibitor, such as zimlovisertib.

In some embodiments, the additional therapeutic agent is a JAK inhibitor, for example the additional therapeutic agent is baricitinib, filgotinib, jaktinib, tofacitinib, or nezulcitinib (TD-0903).

In some embodiments, the additional therapeutic agent is a neutrophil elastase inhibitor, such as alvelestat.

In some embodiments, the additional therapeutic agent is a lung surfactant associated protein D modulator, such as AT-100.

In some embodiments, the additional therapeutic agent is a plasma kallikrein inhibitor, such as donidalorsen.

In some embodiments, the additional therapeutic agent is a lysine specific histone demethylase 1/MAO B inhibitor, such as vafidemstat.

In some embodiments, the additional therapeutic agent is a Mannan-binding lectin serine protease inhibitor, such as conestat alfa.

In some embodiments, the additional therapeutic agent is a maxi K potassium channel inhibitor, such as ENA-001.

In some embodiments, the additional therapeutic agent is a MEK protein kinase inhibitor, such as zapnometinib.

In some embodiments, the additional therapeutic agent is a MEK-1 protein kinase inhibitor/Ras gene inhibitor, such as antroquinonol.

In some embodiments, the additional therapeutic agent is a melanocortin MC1 receptor agonist, such as PL-8177

In some embodiments, the additional therapeutic agent is a melanocortin MC1/MC3 receptor agonist, such as resomelagon acetate.

In some embodiments, the additional therapeutic agent is a matrix metalloprotease-12 inhibitor, such as FP-025.

In some embodiments, the additional therapeutic agent is a NACHT LRR PYD domain protein 3 inhibitor, such as dapansutrile, DFV-890, or ZYIL-1.

In some embodiments, the additional therapeutic agent is a NADPH oxidase inhibitor, such as isuzinaxib.

In some embodiments, the additional therapeutic agent is a neuropilin 2 modulator, such as efzofitimod.

In some embodiments, the additional therapeutic agent is an NK1 receptor antagonist, such as aprepitant or tradipitant.

In some embodiments, the additional therapeutic agent is an NMDA receptor antagonist, such as transcrocetin or ifenprodil.

In some embodiments, the additional therapeutic agent is a nuclear factor kappa B inhibitor/p38 MAP kinase inhibitor, such as zenuzolac.

In some embodiments, the additional therapeutic agent is an ornithine decarboxylase inhibitor, such as eflornithine.

In some embodiments, the additional therapeutic agent is an opioid receptor sigma antagonist 1, such as MR-309.

In some embodiments, the additional therapeutic agent is a PGD2 antagonist, such as asapiprant.

In some embodiments, the additional therapeutic agent is a PDGF receptor antagonist/TGF beta receptor antagonist/p38 MAP kinase inhibitor, such as deupirfenidone.

In some embodiments, the additional therapeutic agent is a phospholipase A2 inhibitor, such as varespladib methyl.

In some embodiments, the additional therapeutic agent is a phosphoinositide 3-kinase inhibitor/mTOR complex inhibitor, such as dactolisib.

In some embodiments, the additional therapeutic agent is a mTOR inhibitor, such as sirolimus.

In some embodiments, the additional therapeutic agent is a phosphoinositide-3 kinase delta/gamma inhibitor, such as duvelisib.

In some embodiments, the additional therapeutic agent is a PIKfyve inhibitor, such as VRG-101.

In some embodiments, the additional therapeutic agent is a plasminogen activator inhibitor 1 inhibitor, such as TM-5614.

In some embodiments, the additional therapeutic agent is a protein tyrosine phosphatase beta inhibitor, such as razuprotafib.

In some embodiments, the additional therapeutic agent is a RIP-1 kinase inhibitor, such as eclitasertib (DNL-758) or SIR-0365.

In some embodiments, the additional therapeutic agent is a Rev protein modulator, such as obefazimod.

In some embodiments, the additional therapeutic agent is an S phase kinase associated protein 2 inhibitor, such as niclosamide, CP-COV3, SCAI-502 or DWRX-2003.

In some embodiments, the additional therapeutic agent is a signal transducer CD24 stimulator, such as EXO-CD24.

In some embodiments, the additional therapeutic agent is a sodium glucose transporter-2 inhibitor, such as dapagliflozin propanediol.

In some embodiments, the additional therapeutic agent is a sodium channel stimulator, such as solnatide.

In some embodiments, the additional therapeutic agent is a sphingosine-1-phosphate receptor-1 agonist/sphingosine-1-phosphate receptor-5 agonist, such as ozanimod.

In some embodiments, the additional therapeutic agent is a non-steroidal anti-inflammatory drug, such as Ampion.

In some embodiments, the additional therapeutic agent is a superoxide dismutase stimulator, such as avasopasem manganese.

In some embodiments, the additional therapeutic agent is a Syk tyrosine kinase inhibitor, such as fostamatinib disodium.

In some embodiments, the additional therapeutic agent is a Tie2 tyrosine kinase receptor agonist, such as AV-001.

In some embodiments, the additional therapeutic agent is a TGFB2 gene inhibitor, such as trabedersen.

In some embodiments, the additional therapeutic agent is a tissue factor inhibitor, such as AB-201.

In some embodiments, the additional therapeutic agent is a TLR-3 agonist, such as rintatolimod.

In some embodiments, the additional therapeutic agent is a TLR-4 antagonist, such as ApTLR-4FT, EB-05, or eritoran.

In some embodiments, the additional therapeutic agent is a TLR-7/8 antagonist, such as enpatoran.

In some embodiments, the additional therapeutic agent is a TLR-2/6 agonist, such as INNA-051.

In some embodiments, the additional therapeutic agent is a TLR-7 agonist, such as PRTX-007 or APR-002.

In some embodiments, the additional therapeutic agent is a TLR agonist, such as PUL-042.

In some embodiments, the additional therapeutic agent is a TLR-4 agonist, such as REVTx-99.

In some embodiments, the additional therapeutic agent is a TLR-2/4 antagonist, such as VB-201.

In some embodiments, the additional therapeutic agent is a TNF alpha ligand inhibitor, such as pegipanermin.

In some embodiments, the additional therapeutic agent is a type I IL-1 receptor antagonist, such as anakinra.

In some embodiments, the additional therapeutic agent is a TREM receptor 1 antagonist, such as nangibotide.

In some embodiments, the additional therapeutic agent is a trypsin inhibitor, such as ulinastatin.

In some embodiments, the additional therapeutic agent is a tubulin inhibitor such as sabizabulin, CCI-001, PCNT-13, CR-42-24, albendazole, entasobulin, SAR-132885, or ON-24160.

In some embodiments, the additional therapeutic agent is a VIP receptor agonist, such as aviptadil.

In some embodiments, the additional therapeutic agent is a xanthine oxidase inhibitor, such as oxypurinol.

In some embodiments, the additional therapeutic agent is a vasodilator, such as iloprost, epoprostenol (VentaProst), zavegepant, TXA-127, USB-002, ambrisentan, nitric oxide nasal spray (NORS), pentoxifylline, propranolol, RESP301, sodium nitrite, or dipyridamole.

In some embodiments, the additional therapeutic agent is a vitamin D3 receptor agonist, such as cholecalciferol.

In some embodiments, the additional therapeutic agent is a zonulin inhibitor, such as larazotide acetate.

In some embodiments, the additional therapeutic agent is a synthetic retinoid derivative, such as fenretinide.

In some embodiments, the additional therapeutic agent is a glucose metabolism inhibitor such as WP-1122 or WP-1096.

In some embodiments, the additional therapeutic agent is adalimumab, AT-H201, 2-deoxy-D-glucose, AD-17002, AIC-649, AMTX-100, astodrimer, AZD-1656, belapectin, bitespiramycin, bucillamine, budesonide, CNM-AgZn-17, Codivir, CT-38, danicopan, didodecyl methotrexate, DW-2008S (DW-2008), EDP-1815, EG-009A, Fabencov, Gamunex, genistein, GLS-1200, hzVSF-v13, imidazolyl ethanamide pentandioic acid, IMM-101, MAS-825, MRG-001, Nasitrol, Nylexa, olverembatinib, OP-101, OPN-019, Orynotide rhesus theta defensin-1, pyronaridine+artesunate, dapsone, RPH-104, sodium pyruvate, Sulforadex, tafenoquine, TB-006, telacebec, Tempol, TL-895, thimesoral, trimodulin, XC-221, XC-7, zunsemetinib, metformin glycinate, lucinactant, EOM-613, mosedipimod, ivermectin, leflunomide, ibudilast, RBT-9, raloxifene, prothione, gemcabene, or idronoxil.

In some embodiments, the additional therapeutic agent is a CD73 antagonist, such as AK-119.

In some embodiments, the additional therapeutic agent is a CD95 protein fusion, such as asunercept.

In some embodiments, the additional therapeutic agent is a complement factor C₂ modulator, such as ARGX-117.

In some embodiments, the additional therapeutic agent is a complement C₃ inhibitor, such as AMY-101 or NGM-621.

In some embodiments, the additional therapeutic agent is a CXC10 chemokine ligand inhibitor, such as EB-06.

In some embodiments, the additional therapeutic agent is a cytotoxic T-lymphocyte protein-4 fusion protein, such as abatacept

In some embodiments, the additional therapeutic agent is an anti-S. Aureus antibody, such as tosatoxumab.

In some embodiments, the additional therapeutic agent is an anti-LPS antibody, such as IMM-124-E.

In some embodiments, the additional therapeutic agent is an adrenomedullin ligand inhibitor, such as enibarcimab.

In some embodiments, the additional therapeutic agent is a basigin inhibitor, such as meplazumab.

In some embodiments, the additional therapeutic agent is a CD3 antagonist, such as foralumab.

In some embodiments, the additional therapeutic agent is a connective tissue growth factor ligand inhibitor, such as PRS-220, pamrevlumab.

In some embodiments, the additional therapeutic agent is a complement C5a factor inhibitor, such as BDB-1 or vilobelimab.

In some embodiments, the additional therapeutic agent is a complement C5 factor inhibitor, such as ravulizumab.

In some embodiments, the additional therapeutic agent is a mannan-binding lectin serine protease-2 inhibitor, such as narsoplimab.

In some embodiments, the additional therapeutic agent is a GM-CSF modulator, such as STSA-1005, gimsilumab, namilumab, plonmarlimab, otilimab, or lenzilumab.

In some embodiments, the additional therapeutic agent is a heat shock protein inhibitor/IL-6 receptor antagonist, such as siltuximab.

In some embodiments, the additional therapeutic agent is an IL-6 receptor antagonist, such as clazakizumab, levilimab, olokizumab, tocilizumab, or sirukumab.

In some embodiments, the additional therapeutic agent is an IL-8 receptor antagonist, such as BMS-986253.

In some embodiments, the additional therapeutic agent is an interleukin-1 beta ligand inhibitor, such as canakinumab.

In some embodiments, the additional therapeutic agent is an interferon gamma ligand inhibitor, such as emapalumab.

In some embodiments, the additional therapeutic agent is an anti-ILT7 antibody, such as daxdilimab.

In some embodiments, the additional therapeutic agent is a monocyte differentiation antigen CD14 inhibitor, such as atibuclimab.

In some embodiments, the additional therapeutic agent is a plasma kallikrein inhibitor, such as lanadelumab.

In some embodiments, the additional therapeutic agent is a platelet glycoprotein VI inhibitor, such as glenzocimab.

In some embodiments, the additional therapeutic agent is a T-cell differentiation antigen CD6 inhibitor, such as itolizumab.

In some embodiments, the additional therapeutic agent is a TNF alpha ligand inhibitor/TNF binding agent, such as infliximab.

In some embodiments, the additional therapeutic agent is an anti-LIGHT antibody, such as AVTX-002.

In some embodiments, the additional therapeutic agent is IMC-2 (valacyclovir+celecoxib), or AXA-1125.

In some embodiments, the additional therapeutic agent is COVID-HIG.

In some embodiments, a compound of the disclosure, or a pharmaceutically acceptable salt thereof, is co-administered with one or more agents useful for the treatment and/or prophylaxis of COVID-19.

Non-limiting examples of such agents include corticosteroids, such as dexamethasone, hydrocortisone, methylprednisolone, or prednisone; interleukin-6 (IL-6) receptor blockers, such as tocilizumab or sarilumab; Janus kinase (JAK) inhibitors, such as baricitinib, ruxolitinib, or tofacitinib; and antiviral agents, such as molnupiravir, sotrovimab, or remdesivir.

In further embodiments, a compound of the disclosure, or a pharmaceutically acceptable salt thereof, is co-administered with two or more agents useful for the treatment of COVID-19. Agents useful for the treatment and/or prophylaxis of COVID-19 include but are not limited to a compound of the disclosure and two additional therapeutic agents, such as nirmatrelvir and ritonavir, casirivimab and imdevimab, or ruxolitinib and tofacitinib.

In some embodiments, the additional therapeutic agent is an antiviral agent. In some embodiments, the antiviral agent is an entry inhibitor. In some embodiments, the antiviral agent is a protease inhibitor. In some embodiments, the antiviral agent is an RNA polymerase inhibitor. In some embodiments, the additional therapeutic agent is an RNA-dependent RNA polymerase (RdRp) inhibitor.

In some embodiments, the antiviral agent is selected from angiotensin converting enzyme 2 inhibitors, angiotensin converting enzyme 2 modulators, angiotensin converting enzyme 2 stimulators, angiotensin II AT-2 receptor agonists, angiotensin II AT-2 receptor antagonists, angiotensin II receptor modulators, coronavirus nucleoprotein modulators, coronavirus small envelope protein modulators, coronavirus spike glycoprotein inhibitors, coronavirus spike glycoprotein modulators, SARS-CoV-2 envelope small membrane protein inhibitors, SARS-CoV-2 envelope small membrane protein modulators, SARS-CoV-2 MPro inhibitors, SARS-CoV-2 non structural protein 8 modulators, SARS-CoV-2 nucleoprotein inhibitors, SARS-CoV-2 nucleoprotein modulators, SARS-CoV-2 protein 3a inhibitors, SARS-CoV-2 replicase polyprotein 1a inhibitors, SARS-CoV-2 replicase polyprotein 1a modulators, SARS-CoV-2 replicase polyprotein 1ab inhibitors, SARS-CoV-2 replicase polyprotein 1ab modulators, SARS-CoV-2 spike glycoprotein inhibitors, SARS-CoV-2 spike glycoprotein modulators, SARS-CoV-2 structural glycoprotein modulators, papain inhibitors, protease inhibitors, protease modulators, RNA polymerase inhibitors, RNA polymerase modulators, RNA-dependent RNA polymerase (RdRp) inhibitors, SARS coronavirus 3C protease like inhibitors, SARS-CoV-2 nsp14 methyltransferase enzyme inhibitor, 3CLpro/Mpro inhibitors, serine protease inhibitors, transmembrane serine protease 2 inhibitors, transmembrane serine protease 2 modulators, viral envelope protein inhibitors, viral protease inhibitors, viral protease modulators, viral protein target modulators, viral ribonuclease inhibitors, and viral structural protein modulators.

In some embodiments, the additional therapeutic agent is an entry inhibitor. For example, in some embodiments the additional therapeutic agent is an ACE2 inhibitor, a fusion inhibitor, or a protease inhibitor.

In some embodiments, the additional therapeutic agent is an angiotensin converting enzyme 2 inhibitor, such as SBK-001.

In some embodiments, the additional therapeutic agent is an angiotensin converting enzyme 2 modulator, such as neumifil or JN-2019.

In some embodiments, the additional therapeutic agent is an entry inhibitor such as MU-UNMC-1, or SAI-4.

In some embodiments, the additional therapeutic agent is an angiotensin converting enzyme 2 stimulator, such as alunacedase alfa.

In some embodiments, the additional therapeutic agent is an angiotensin II AT-2 receptor agonist, such as VP-01.

In some embodiments, the additional therapeutic agent is an ACE II receptor antagonist, such as DX-600.

In some embodiments, the additional therapeutic agent is an angiotensin II receptor modulator, such as TXA-127.

In some embodiments, the additional therapeutic agent is a transmembrane serine protease 2 modulator, such as BC-201, N-0385.

In some embodiments, the additional therapeutic agent is a viral envelope protein inhibitor, such as MXB-9 or MXB-004.

In some embodiments, the additional therapeutic agent is a RNAi agent such as ARO-COV or SNS-812.

In some embodiments, the additional therapeutic agent is a vaccine. For example, in some embodiments, the additional therapeutic agent is a DNA vaccine, RNA vaccine, live-attenuated vaccine, inactivated vaccine (i.e., inactivated SARS-CoV-2 vaccine), therapeutic vaccine, prophylactic vaccine, protein-based vaccine, viral vector vaccine, cellular vaccine, or dendritic cell vaccine.

In some embodiments, the additional therapeutic agent is a vaccine such as tozinameran, NVX-CoV2373, elasomeran, KD-414, Ad26.COV2-S, Vaxzevria, SCB-2019, AKS-452, VLA-2001, HDT-301, S-268019, MVC-COV1901, mRNA-1273.214, mRNA-1273.213, mRNA-1273.222, NVX-CoV2515, Covaxin, BBIBP-CorV, GBP-510, mRNA-1273.351+mRNA-1273.617 (SARS-CoV-2 multivalent mRNA vaccine, COVID-19), Ad5-nCoV, Omicron-based COVID-19 vaccine (mRNA vaccine, COVID-19), mRNA-1073, mRNA-1273.214, mRNA-1230, mRNA-1283, Omicron-based COVID-19 vaccine, SARS-CoV-2 Protein Subunit Recombinant Vaccine, Sputnik M, ZyCoV-D, COVID-19 XWG-03, mRNA-1273.529, mRNA-1010, CoronaVac, AZD-2816, Sputnik V, inactivated SARS-CoV-2 vaccine (Vero cell, COVID-19), DS-5670, PHH-1V, INO-4800, UB-612, coronavirus vaccine (whole-virion, inactivated/purified), ReCOV, MT-2766, ARCT-154, SP-0253, CORBEVAX, mRNA-1273.211, ZF-2001, Sputnik Light, recombinant protein vaccine (COVID-19/SARS-CoV-2 infection), VSV vector-based vaccine targeting spike glycoprotein (COVID-19), VLA-2101, GRT-R912, GRAd-COV2, VPM-1002, COViran Barekat, Ad5-nCoV-IH, ARCoV, Covax-19, recombinant SARS-CoV-2 vaccine (protein subunit/CHO cell, COVID-19), BBV-154, RAZI Cov Pars, COVID-19 vaccine (inactivated/Vero cells/intramuscular, SARS-CoV-2 infection), COVID-19 vaccine (inactivated, Vero cells/intramuscular), BNT-162b2s01, BNT-162b4, BNT-162b5, BNT-162b2 Omi, BNT-162b2 bivalent, CIGB-66, mRNA-1273.617, Mycobacterium w, ERUCOV-VAC, AG-0301-COVID19, fakhravac, AV-COVID-19, peptide vaccine (COVID-19), Nanocovax, SARS-CoV-2 vaccine (inactivated/Vero cells/intramuscular, COVID-19), QAZCOVID-IN, S-875670 nasal vaccine, VTP-500, or BNT162b5.

In some embodiments, the additional therapeutic agent is a protease inhibitor. For example, in some embodiments the additional therapeutic agent is a 3C-like cysteine protease inhibitor (3CLpro, also called Main protease, Mpro), a papain-like protease inhibitor (PLpro), serine protease inhibitor, or transmembrane serine protease 2 inhibitor (TMPRSS2).

In some embodiments, the additional therapeutic agent is a 3CLpro/Mpro inhibitor, such as ABBV-903, AB-343, CDI-873, GC-373, GC-376, pomotrelvir (PBI-0451), UCI-1, bofutrelvir (FB-2001, DC-402234), DC-402267, GDI-4405, HS-10517, RAY-1216, MPI-8, SH-879, SH-580, EDP-235, VV-993, CDI-988, MI-30, nirmatrelvir, ensitrelvir, ASC-11, ASC-11+ritonavir, EDDC-2214, SIM-0417, PF-07817883, simnotrelvir, simnotrelvir+ritonavir, SYH-2055, ISM-3312, CDI-45205, LHP-803 (COR-803), ALG-097111, TJC-642, CVD-0013943, olgotrelvir (STI-1558), eravacycline, cynarine, WPV-01, or prexasertib.

In some embodiments, the additional therapeutic agent is a papain-like protease inhibitor (PLpro), such as SBFM-PL4 or GRL-0617.

In some embodiments, the additional therapeutic agent is a SARS-CoV-2 helicase Nsp13 inhibitor, such as EIS-4363.

In some embodiments, the additional therapeutic agent is a SARS-CoV-2 helicase Nsp14 inhibitor, such as TO-507.

In some embodiments, the additional therapeutic agent is a SARS-CoV-2 spike (S) and protease modulator, such as ENU-200.

In some embodiments, the additional therapeutic agent is a protease inhibitor, such as ALG-097558 or MRX-18.

In some embodiments, the additional therapeutic agent is a serine protease inhibitor, such as upamostat, nafamostat, camostat mesylate, nafamostat mesylate, or camostat.

In some embodiments, the additional therapeutic agent is a 3CLpro/transmembrane serine protease 2 inhibitor, such as SNB-01 (pentarlandir) or SNB-02.

In some embodiments, the additional therapeutic agent is a viral protease inhibitor, such as Pan-Corona, Cov-X, or bepridil.

In some embodiments, the additional therapeutic agent is an RNA polymerase inhibitor. For example, in some embodiments, the additional therapeutic agent is an RNA polymerase inhibitor, or an RNA-dependent RNA polymerase (RdRp) inhibitor.

In some embodiments, the additional therapeutic agent is an RNA-dependent RNA polymerase (RdRp) inhibitor, such as remdesivir, NV-CoV-2, NV-CoV-2-R, NV-CoV-1 encapsulated remdesivir, CMX-8521, GS-621763, GS-5245, GS-441524, DEP remdesivir, ATV-006, deuremidevir (VV-116), LGN-20, CMX-521, SHEN-26, MB-905, and compounds disclosed in WO2022142477, WO2021213288, WO2022047065.

In some embodiments, the additional therapeutic agent is an RNA polymerase inhibitor, such as molnupiravir (EIDD-2801), favipiravir, bemnifosbuvir, sofosbuvir, ASC-10, or galidesivir.

In some embodiments, the additional therapeutic agent is viral entry inhibitor, such as brilacidin.

In some embodiments, the additional therapeutic agent is an antibody that binds to a coronavirus, for example an antibody that binds to SARS or MERS.

In some embodiments, the additional therapeutic agent is an antibody, for example a monoclonal antibody. For example, the additional therapeutic agent is an antibody against SARS-CoV-2, neutralizing nanobodies, antibodies that target the SARS-CoV-2 spike protein, fusion proteins, multispecific antibodies, and antibodies that can neutralize SARS-CoV-2 (SARS-CoV-2 neutralizing antibodies).

In some embodiments, the additional therapeutic agent is an antibody that targets specific sites on ACE2. In some embodiments, the additional therapeutic agent is a polypeptide targeting SARS-CoV-2 spike protein (S-protein).

In some embodiments, the additional therapeutic agent is a SARS-CoV-2 virus antibody.

In some embodiments, the antibody is ABBV-47D11, COVI-GUARD (STI-1499), C144-LS+C135-LS, DIOS-202, DIOS-203, DIOS-301, DXP-604, JMB-2002, LY-CovMab, bamlanivimab (LY-CoV555), GIGA-2050, IBI-314, S309, SAB-185, etesevimab (CB6), COR-101, JS016, VNAR, VIR-7832 and/or sotrovimab (VIR-7831), casirivimab+imdevimab (REGN-COV2 or REGN10933+RGN10987), BAT2020, BAT2019, 47D11, YBSW-015, or PA-001.

In some embodiments, the additional therapeutic agent is STI-9199 (COVI-SHIELD), STI-9167 or AR-701 (AR-703 and AR-720).

In some embodiments, the additional therapeutic agent is BRII-196, BRII-198, ADG-10, adintrevimab (ADG-20), ABP-300, BA-7208, BI-767551, BHV-1200, CT-P63, JS-026, sotrovimab (GSK-4182136), tixagevimab+cilgavimab (AZD-7442), regdanvimab, SAB-301, AOD-01, plutavimab (COVI-AMG), 9MW-3311 (MW-33), DXP-593, BSVEQAb, anti-SARS-CoV-2 IgY, COVID-EIG, CSL-760, F-61, REGN-3048-3051, SARS-CoV-2 monoclonal antibodies (COVID-19, ADM-03820), enuzovimab (HFB-30132A), INM-005, SCTA01, TY-027, XAV-19, amubarvimab+romlusevimab, SCTA-01, bebtelovimab, beludavimab, IBI-0123, IGM-6268. FYB-207, FS-2101, RBT-0813, REGN-14256, REGN-14284, SPKM-001, XVR-011, TB202-3, TB181-36, TB339-031, LMN-301, LQ-050, COVAB-36, MAD-0004J08, STI-2099, TATX-03, TZLS-501, ZCB-11, AZD-3152, VYD-222, XVR-012, or ACV-200-17.

In some embodiments, the additional therapeutic agent is an engineered ACE-2-IgGl-Fc-fusion protein targeting SARS-Cov-2 RBD, such as EU-129, bivalent ACE2-IgG Fc null fusion protein (SI-F019).

In some embodiments, the additional therapeutic agent is an ACE2-Fc receptor fusion protein, such as HLX-71.

In some embodiments, the additional therapeutic agent is ensovibep.

In some embodiments, the additional therapeutic agent is SYZJ-001.

In some embodiments, the additional therapeutic agent is an HIV-1 protease inhibitor, such as ASC-09F (ASC-09+ritonavir) or lopinavir+ritonavir.

In some embodiments, the additional therapeutic agent is a non-nucleoside reverse transcriptase inhibitor, such as elsulfavirine.

In some embodiments, the additional therapeutic agent is a nucleoside reverse transcriptase inhibitor, such as azvudine.

In some embodiments, the additional therapeutic agent is Abbv-990, ABBV-903, 2b-11, 5-aminolevulinic phosphoric acid, AGP-600, AGM-380, AIP-502, ALG-150150, BAT-2022, NED-260, burfiralimab, ALG-097431, bardoxolone, BW-PS-119, clofoctol, CR-405, delcetravir, D4-102-01, D4-102-02, ESFAM-289, ENOB-CV-01, ENOB-CV-11, EIS-10700, EV-300, beta-521, GEA-001, SIM-0417, molnupiravir, Pan-Corona, Tollovir, nirmatrelvir+ritonavir (Paxlovid®), JTBC-00201, favipiravir, favipiravir+cathepsin inhibitor (TNX-3900), GC-376, upamostat, LeSoleil-O1, LeSoleil-02+, benfovir, VV-116, VV-993, SNB-01, EDP-235, Cov-X, ensitrelvir, MPI-8, masitinib, ALG-097558, ASC-11, PBI-0451, nafamostat, nafamostat mesylate, CDI-45205, LHP-803 (COR-803), ALG-097111, BC-201, SH-879, CDI-873, CDI-988, remdesivir, NV-CoV-2-R, NV-CoV-1 encapsulated remdesivir, NA-831+remdesivir, DEP remdesivir, GS-621763, GS-5245, GLS-5310, bemnifosbuvir, QLS-1128, ASC-10, SBFM-PL4, camostat mesylate, UCI-1, FB-2001 (DC-402234), ebselen, SH-580, LeSoleil-01, LeSoleil-02+, MRX-18, MXB-9, MI-09, MI-30, SNB-02, SJP-002C, TJC-642, ENU-200, CVD-0013943, GS-441524, bepridil, MXB-004, eravacycline, GRL-0617, GST-HG171, GST-HG171+ritonavir, camostat, GC-373, KD-1, nitazoxanide, cynarine, prexasertib, PL-M, RAY-1216, SACT-COVID-19, MP-18, EIDD-1931, EDDC-2214, nitric oxide, apabetalone, AnQlar, SBK-001, LQ-050, CG-SpikeDown, bamlanivimab, JTBC-00101, HLX-71, HT-002, HY-209, HY-3000, HSC-1553, FYB-207, ensovibep, SYZJ-001, EU-129, neumifil, JN-2019 (KG-2019), LCB-99, AR-701, vostesyl, PLM-402, PJS-539, CTB-ACE2, TB181-36, TB202-3, ABP-300, XVR-011, MSP-008-22, MU-UNMC-1, MU-UNMC-2, MIC-1930, MLT-103, Mpro inhibitors (Anixa Biosciences), PBF-4554, alunacedase alfa, VP-01, TRV-027, DX-600, TXA-127, NVX-CoV2515, raphamin, RCYM-002, RCYM-003, riamilovir, SARS-Cov-2 PL pro inhibitor (Enanta), SBP-502, SM-4, STB-R040, THY-01, tozinameran, elasomeran, Ad5-nCoV, BBIBP-CorV, CoronaVac, MVC-COV1901, NVX-CoV2373, sotrovimab, Sputnik V, TEE-001, Tyme-19, Vaxzevria, XW-001, ZF-2001, or ZyCoV-D.

In some embodiments, the additional therapeutic or prophylactic agent is a SARS-CoV-2 MPro inhibitor. In some embodiments, the SARS-CoV-2 MPro inhibitor is nirmatrelvir. In some embodiments, the SARS-CoV-2 MPro inhibitor is ritonavir.

It is also possible to combine any compound of the invention with one or more additional active therapeutic agents in a unitary dosage form for simultaneous or sequential administration to a patient. The combination therapy may be administered as a simultaneous or sequential regimen. When administered sequentially, the combination may be administered in two or more administrations.

Co-administration of a compound of the invention with one or more other active therapeutic agents generally refers to simultaneous or sequential administration of a compound of the invention and one or more other active therapeutic agents, such that therapeutically effective amounts of the compound of the invention and one or more other active therapeutic agents are both present in the body of the patient.

Co-administration includes administration of unit dosages of the compounds described herein before or after administration of unit dosages of one or more other active therapeutic agents, for example, administration of the compounds described herein within seconds, minutes, or hours of the administration of one or more other active therapeutic agents. For example, a unit dose of a compound of the invention can be administered first, followed within seconds or minutes by administration of a unit dose of one or more other active therapeutic agents. Alternatively, a unit dose of one or more other therapeutic agents can be administered first, followed by administration of a unit dose of a compound of the invention within seconds or minutes. In some cases, it may be desirable to administer a unit dose of a compound of the invention first, followed, after a period of hours (e.g., 1-12 hours), by administration of a unit dose of one or more other active therapeutic agents. In other cases, it may be desirable to administer a unit dose of one or more other active therapeutic agents first, followed, after a period of hours (e.g., 1-12 hours), by administration of a unit dose of a compound of the invention.

The combination therapy may provide “synergy” and “synergistic”, i.e., the effect achieved when the active ingredients used together is greater than the sum of the effects that results from using the compounds separately. A synergistic effect may be attained when the active ingredients are: (1) co-formulated and administered or delivered simultaneously in a combined formulation; (2) delivered by alternation or in parallel as separate formulations; or (3) by some other regimen. When delivered in alternation therapy, a synergistic effect may be attained when the compounds are administered or delivered sequentially, e.g., in separate tablets, pills or capsules, or by different injections in separate syringes. In general, during alternation therapy, an effective dosage of each active ingredient is administered sequentially, i.e., serially, whereas in combination therapy, effective dosages of two or more active ingredients are administered together. A synergistic anti-viral effect denotes an antiviral effect, which is greater than the predicted purely additive effects of the individual compounds of the combination.

The compounds described herein are also used in combination with other active therapeutic agents. For the treatment of Pneumoviridae virus infections, preferably, the other active therapeutic agent is active against Pneumoviridae virus infections, particularly respiratory syncytial virus infections and/or metapneumovirus infections. Non-limiting examples of these other active therapeutic agents active against RSV are ribavirin, palivizumab, motavizumab, RSV-IGIV (RespiGam®), MEDI-557, A-60444 (also known as RSV604), MDT-637, BMS-433771, ALN-RSVO, ALX-0171 and mixtures thereof. Other non-limiting examples of other active therapeutic agents active against respiratory syncytial virus infections include respiratory syncytial virus protein F inhibitors, such as AK-0529; RV-521, ALX-0171, JNJ-53718678, BTA-585, and presatovir; RNA polymerase inhibitors, such as lumicitabine and ALS-8112; anti-RSV G protein antibodies, such as anti-G-protein mAb; viral replication inhibitors, such as nitazoxanide.

In some embodiments, the other active therapeutic agent may be a vaccine for the treatment or prevention of RSV, including but not limited to MVA-BN RSV, RSV-F, MEDI-8897, JNJ-64400141, DPX-RSV, SynGEM, GSK-3389245A, GSK-300389-1A, RSV-MEDI deltaM2-2 vaccine, VRC-RSVRGPO84-00VP, Ad35-RSV-FA2, Ad26-RSV-FA2, and RSV fusion glycoprotein subunit vaccine.

Non-limiting examples of other active therapeutic agents active against metapneumovirus infections include sialidase modulators such as DAS-181; RNA polymerase inhibitors, such as ALS-8112; and antibodies for the treatment of Metapneumovirus infections, such as EV-046113.

In some embodiments, the other active therapeutic agent may be a vaccine for the treatment or prevention of metapneumovirus infections, including but not limited to mRNA-1653 and rHMPV-Pa vaccine.

The compounds described herein are also used in combination with other active therapeutic agents. For the treatment ofpicornaviridae virus infections, preferably, the other active therapeutic agent is active against picornaviridae virus infections, particularly Enterovirus infections. Non-limiting examples of these other active therapeutic agents are capsid binding inhibitors such as pleconaril, BTA-798 (vapendavir) and other compounds disclosed by Wu, et al. (U.S. Pat. No. 7,078,403) and Watson (U.S. Pat. No. 7,166,604); fusion sialidase protein such as DAS-181; a capsid protein VP1 inhibitor such as VVX-003 and AZN-001; a viral protease inhibitor such as CW-33; a phosphatidylinositol 4 kinase beta inhibitor such as GSK-480 and GSK-533; anti-EV71 antibody.

In some embodiments, the other active therapeutic agent may be a vaccine for the treatment or prevention ofpicornaviridae virus infections, including but not limited to EV71 vaccines, TAK-021, and EV-D68 adenovector-based vaccine.

Many of the infections of the pneumoviridae, picornaviridae, and coronaviridae viruses are respiratory infections. Therefore, additional active therapeutics used to treat respiratory symptoms and sequelae of infection may be used in combination with the compounds described herein. The additional agents are preferably administered orally or by direct inhalation. For example, other preferred additional therapeutic agents in combination with the compounds described herein for the treatment of viral respiratory infections include, but are not limited to, bronchodilators and corticosteroids.

Glucocorticoids, which were first introduced as an asthma therapy in 1950 (Carryer, Journal of Allergy, 21, 282-287, 1950), remain the most potent and consistently effective therapy for this disease, although their mechanism of action is not yet fully understood (Morris, J. Allergy Clin. Immunol., 75 (1 Pt) 1-13, 1985). Unfortunately, oral glucocorticoid therapies are associated with profound undesirable side effects such as truncal obesity, hypertension, glaucoma, glucose intolerance, acceleration of cataract formation, bone mineral loss, and psychological effects, all of which limit their use as long-term therapeutic agents (Goodman and Gilman, 10th edition, 2001). A solution to systemic side effects is to deliver steroid drugs directly to the site of inflammation. Inhaled corticosteroids (ICS) have been developed to mitigate the severe adverse effects of oral steroids. Non-limiting examples of corticosteroids that may be used in combinations with the compounds described herein are dexamethasone, dexamethasone sodium phosphate, fluorometholone, fluorometholone acetate, loteprednol, loteprednol etabonate, hydrocortisone, prednisolone, fludrocortisones, triamcinolone, triamcinolone acetonide, betamethasone, beclomethasone diproprionate, methylprednisolone, fluocinolone, fluocinolone acetonide, flunisolide, fluocortin-21-butylate, flumethasone, flumetasone pivalate, budesonide, halobetasol propionate, mometasone furoate, fluticasone, AZD-7594, ciclesonide; or a pharmaceutically acceptable salts thereof.

Other anti-inflammatory agents working through anti-inflammatory cascade mechanisms are also useful as additional therapeutic agents in combination with the compounds described herein for the treatment of viral respiratory infections. Applying “anti-inflammatory signal transduction modulators” (referred to in this text as AIS™), like phosphodiesterase inhibitors (e.g., PDE-4, PDE-5, or PDE-7 specific), transcription factor inhibitors (e.g., blocking NFκB through TKK inhibition), or kinase inhibitors (e.g., blocking P38 MAP, INK, PI3K, EGFR or Syk) is a logical approach to switching off inflammation as these small molecules target a limited number of common intracellular pathways—those signal transduction pathways that are critical points for the anti-inflammatory therapeutic intervention (see review by P. J. Barnes, 2006). These non-limiting additional therapeutic agents include: 5-(2,4-Difluoro-phenoxy)-1-isobutyl-1H-indazole-6-carboxylic acid (2-dimethylamino-ethyl)-amide (P38 Map kinase inhibitor ARRY-797); 3-Cyclopropylmethoxy-N-(3,5-dichloro-pyridin-4-yl)-4-difluorormethoxy-benzamide (PDE-4 inhibitor Roflumilast); 4-[2-(3-cyclopentyloxy-4-methoxyphenyl)-2-phenyl-ethyl]-pyridine (PDE-4 inhibitor CDP-840); N-(3,5-dichloro-4-pyridinyl)-4-(difluoromethoxy)-8-[(methylsulfonyl)amino]-1-dibenzofurancarboxamide (PDE-4 inhibitor Oglemilast); N-(3,5-Dichloro-pyridin-4-yl)-2-[1-(4-fluorobenzyl)-5-hydroxy-1H-indol-3-yl]-2-oxo-acetamide (PDE-4 inhibitor AWD 12-281); 8-Methoxy-2-trifluoromethyl-quinoline-5-carboxylic acid (3,5-dichloro-1-oxy-pyridin-4-yl)-amide (PDE-4 inhibitor Sch 351591); 4-[5-(4-Fluorophenyl)-2-(4-methanesulfinyl-phenyl)-1H-imidazol-4-yl]-pyridine (P38 inhibitor SB-203850); 4-[4-(4-Fluoro-phenyl)-1-(3-phenyl-propyl)-5-pyridin-4-yl-1H-imidazol-2-yl]-but-3-yn-1-ol (P38 inhibitor RWJ-67657); 4-Cyano-4-(3-cyclopentyloxy-4-methoxy-phenyl)-cyclohexanecarboxylic acid 2-diethylamino-ethyl ester (2-diethyl-ethyl ester prodrug of Cilomilast, PDE-4 inhibitor); (3-Chloro-4-fluorophenyl)-[7-methoxy-6-(3-morpholin-4-yl-propoxy)-quinazolin-4-yl]-amine (Gefitinib, EGFR inhibitor); and 4-(4-Methyl-piperazin-1-ylmethyl)-N-[4-methyl-3-(4-pyridin-3-yl-pyrimidin-2-ylamino)-phenyl]-benzamide (Imatinib, EGFR inhibitor).

Combinations comprising inhaled β2-adrenoreceptor agonist bronchodilators such as formoterol, albuterol or salmeterol with the compounds described herein are also suitable, but non-limiting, combinations useful for the treatment of respiratory viral infections.

Combinations of inhaled β2-adrenoreceptor agonist bronchodilators such as formoterol or salmeterol with ICS's are also used to treat both the bronchoconstriction and the inflammation (Symbicort® and Advair®, respectively). The combinations comprising these ICS and β2-adrenoreceptor agonist combinations along with the compounds described herein are also suitable, but non-limiting, combinations useful for the treatment of respiratory viral infections.

Other examples of Beta 2 adrenoceptor agonists are bedoradrine, vilanterol, indacaterol, olodaterol, tulobuterol, formoterol, abediterol, salbutamol, arformoterol, levalbuterol, fenoterol, and TD-5471.

For the treatment or prophylaxis of pulmonary broncho-constriction, anticholinergics are of potential use and, therefore, useful as an additional therapeutic agent in combination with the compounds described herein for the treatment of viral respiratory infections. These anticholinergics include, but are not limited to, antagonists of the muscarinic receptor (particularly of the M3 subtype) which have shown therapeutic efficacy in man for the control of cholinergic tone in COPD (Witek, 1999); 1-{4-Hydroxy-1-[3,3,3-tris-(4-fluoro-phenyl)-propionyl]-pyrrolidine-2-carbonyl}-pyrrolidine-2-carboxylic acid (1-methyl-piperidin-4-ylmethyl)-amide; 3-[3-(2-Diethylamino-acetoxy)-2-phenyl-propionyloxy]-8-isopropyl-8-methyl-8-azonia-bicyclo[3.2.1]octane (Ipratropium-N,N-diethylglycinate); 1-Cyclohexyl-3,4-dihydro-1H-isoquinoline-2-carboxylic acid 1-aza-bicyclo[2.2.2]oct-3-yl ester (Solifenacin); 2-Hydroxymethyl-4-methanesulfinyl-2-phenyl-butyric acid 1-aza-bicyclo[2.2.2]oct-3-yl ester (Revatropate); 2-{1-[2-(2,3-Dihydro-benzofuran-5-yl)-ethyl]-pyrrolidin-3-yl}-2,2-diphenyl-acetamide (Darifenacin); 4-Azepan-1-yl-2,2-diphenyl-butyramide (Buzepide); 7-[3-(2-Diethylamino-acetoxy)-2-phenyl-propionyloxy]-9-ethyl-9-methyl-3-oxa-9-azonia-tricyclo[3.3.1.02,4]nonane (Oxitropium-N,N-diethylglycinate); 7-[2-(2-Diethylamino-acetoxy)-2,2-di-thiophen-2-yl-acetoxy]-9,9-dimethyl-3-oxa-9-azonia-tricyclo[3.3.1.02,4]nonane (Tiotropium-N,N-diethylglycinate); Dimethylamino-acetic acid 2-(3-diisopropylamino-1-phenyl-propyl)-4-methyl-phenyl ester (Tolterodine-N,N-dimethylglycinate); 3-[4,4-Bis-(4-fluoro-phenyl)-2-oxo-imidazolidin-1-yl]-1-methyl-1-(2-oxo-2-pyridin-2-yl-ethyl)-pyrrolidinium; 1-[1-(3-Fluoro-benzyl)-piperidin-4-yl]-4,4-bis-(4-fluoro-phenyl)-imidazolidin-2-one; 1-Cyclooctyl-3-(3-methoxy-1-aza-bicyclo[2.2.2]oct-3-yl)-1-phenyl-prop-2-yn-1-ol; 3-[2-(2-Diethylamino-acetoxy)-2,2-di-thiophen-2-yl-acetoxy]-1-(3-phenoxy-propyl)-1-azonia-bicyclo[2.2.2]octane (Aclidinium-N,N-diethylglycinate); or (2-Diethylamino-acetoxy)-di-thiophen-2-yl-acetic acid 1-methyl-1-(2-phenoxy-ethyl)-piperidin-4-yl ester; revefenacin, glycopyrronium bromide, umeclidinium bromide, tiotropium bromide, aclidinium bromide, bencycloquidium bromide.

The compounds described herein may also be combined with mucolytic agents to treat both the infection and symptoms of respiratory infections. A non-limiting example of a mucolytic agent is ambroxol. Similarly, the compounds may be combined with expectorants to treat both the infection and symptoms of respiratory infections. A non-limiting example of an expectorant is guaifenesin.

Nebulized hypertonic saline is used to improve immediate and long-term clearance of small airways in patients with lung diseases (Kuzik, J. Pediatrics 2007, 266). Thus, the compounds described herein may also be combined with nebulized hypertonic saline particularly when the virus infection is complicated with bronchiolitis. The combination of the compound described herein with hypertonic saline may also comprise any of the additional agents discussed above. In one embodiment, nebulized about 3% hypertonic saline is used.

The compounds and compositions provided herein are also used in combination with other active therapeutic agents. For the treatment offlaviviridae virus infections, preferably, the other active therapeutic agent is active againstflaviviridae virus infections.

For treatment of the dengue virus infection, non-limiting examples of the other active therapeutic agents are host cell factor modulators, such as GBV-006; fenretinide ABX-220, BRM-211; alpha-glucosidase 1 inhibitors, such as celgosivir; platelet activating factor receptor (PAFR) antagonists, such as modipafant; cadherin-5/Factor Ia modulators, such as FX-06; NS4B inhibitors, such as JNJ-8359; viral RNA splicing modulators, such as ABX-202; a NS5 polymerase inhibitor; a NS3 protease inhibitor; and a TLR modulator.

In some embodiments, the other active therapeutic agent may be a vaccine for the treatment or prevention of dengue, including but not limited to TetraVax-DV, Dengvaxia®, DPIV-001, TAK-003, live attenuated dengue vaccine, tetravalent dengue fever vaccine, tetravalent DNA vaccine, rDEN2delta30-7169; and DENV-1 PIV.

The compounds described herein are also used in combination with other active therapeutic agents. For the treatment offiloviridae virus infections, preferably, the other active therapeutic agent is active againstfiloviridae virus infections, particularly Marburg virus, Ebola virus and Cueva virus infections. Non-limiting examples of these other active therapeutic agents are: ribavirin, amiodarone, dronedarone, verapamil, Ebola Convalescent Plasma (ECP), TKM-100201, BCX4430 ((2S,3S,4R,5R)-2-(4-amino-5H-pyrrolo[3,2-d]pyrimidin-7-yl)-5-(hydroxymethyl)pyrrolidine-3,4-diol), TKM-Ebola, T-705 monophosphate, T-705 diphosphate, T-705 triphosphate, FGI-106 (1-N,7-N-bis[3-(dimethylamino)propyl]-3,9-dimethylquinolino[8,7-h]quinolone-1,7-diamine), rNAPc2, OS-2966, brincidofovir, remdesivir; RNA polymerase inhibitors, such as galidesivir, favipiravir (also known as T-705 or Avigan), JK-05; host cell factor modulators, such as GMV-006; cadherin-5/factor Ia modulators, such as FX-06; and antibodies for the treatment of Ebola, such as REGN-3470-3471-3479 and ZMapp.

Other non-limiting active therapeutic agents active against Ebola include an alpha-glucosidase 1 inhibitor, a cathepsin B inhibitor, a CD29 antagonist, a dendritic ICAM-3 grabbing nonintegrin 1 inhibitor, an estrogen receptor antagonist, a factor VII antagonist HLA class II antigen modulator, a host cell factor modulator, a Interferon alpha ligand, a neutral alpha glucosidase AB inhibitor, a niemann-Pick C1 protein inhibitor, a nucleoprotein inhibitor, a polymerase cofactor VP35 inhibitor, a Serine protease inhibitor, a tissue factor inhibitor, a TLR-3 agonist, a viral envelope glycoprotein inhibitor, and an Ebola virus entry inhibitors (NPC1 inhibitors).

In some embodiments, the other active therapeutic agent may be a vaccine for the treatment or prevention of Ebola, including but not limited to VRC-EBOADC076-00-VP, adenovirus-based Ebola vaccine, rVSV-EBOV, rVSVN4CT1-EBOVGP, MVA-BN Filo+Ad26-ZEBOV regimen, INO-4212, VRC-EBODNA023-00-VP, VRC-EBOADC069-00-VP, GamEvac-combi vaccine, SRC VB Vector, HPIV3/EboGP vaccine, MVA-EBOZ, Ebola recombinant glycoprotein vaccine, Vaxart adenovirus vector 5-based Ebola vaccine, FiloVax vaccine, GOVX-E301, and GOVX-E302.

The compounds described herein may also be used in combination with phosphoramidate morpholino oligomers (PMOs), which are synthetic antisense oligonucleotide analogs designed to interfere with translational processes by forming base-pair duplexes with specific RNA sequences. Examples of PMOs include but are not limited to AVI-7287, AVI-7288, AVI-7537, AVI-7539, AVI-6002, and AVI-6003.

The compounds described herein are also intended for use with general care provided to patients withfiloviridae viral infections, including parenteral fluids (including dextrose saline and Ringer's lactate) and nutrition, antibiotic (including metronidazole and cephalosporin antibiotics, such as ceftriaxone and cefuroxime) and/or antifungal prophylaxis, fever and pain medication, antiemetic (such as metoclopramide) and/or antidiarrheal agents, vitamin and mineral supplements (including Vitamin K and zinc sulfate), anti-inflammatory agents (such as ibuprofen), pain medications, and medications for other common diseases in the patient population, such anti-malarial agents (including artemether and artesunate-lumefantrine combination therapy), typhoid (including quinolone antibiotics, such as ciprofloxacin, macrolide antibiotics, such as azithromycin, cephalosporin antibiotics, such as ceftriaxone, or aminopenicillins, such as ampicillin), or shigellosis.

Also provided herein is a method of treating a viral infection in a human in need thereof, the method comprising administering to the human
(i) Compound 1:

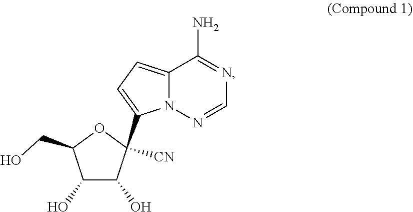 [SMILES: N#C[C@@]1(c2ccc3c(N)ncnn23)O[C@H](CO)[C@@H](O)[C@H]1O]

a deuterated Compound 1, a prodrug of Compound 1, a prodrug of deuterated Compound 1, or a pharmaceutically acceptable salt thereof, and
(ii) one or more SARS-CoV-2 MPro inhibitors;
   wherein when the prodrug of Compound 1, the prodrug of deuterated Compound 1, or the pharmaceutically acceptable salt thereof is administered to the human, the prodrug of Compound 1, the prodrug of deuterated Compound 1, or the pharmaceutically acceptable salt thereof is converted substantially to Compound 1 or deuterated Compound 1.

In some embodiments, the SARS-CoV-2 MPro inhibitor is nirmatrelvir, ritonavir, or combination thereof.

Also provided herein is a method of treating a viral infection in a human in need thereof, the method comprising administering to the human
(i) Compound 1:

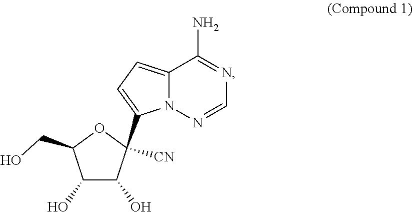 [SMILES: N#C[C@@]1(c2ccc3c(N)ncnn23)O[C@H](CO)[C@@H](O)[C@H]1O]

a deuterated Compound 1, a prodrug of Compound 1, a prodrug of deuterated Compound 1, or a pharmaceutically acceptable salt thereof, and
(ii) nirmatrelvir, ritonavir, or a combination of nirmatrelvir and ritonavir;
   wherein when the prodrug of Compound 1, the prodrug of deuterated Compound 1, or the pharmaceutically acceptable salt thereof is administered to the human, the prodrug of Compound 1, the prodrug of deuterated Compound 1, or the pharmaceutically acceptable salt thereof is converted substantially to Compound 1 or deuterated Compound 1.

In some embodiments, the method comprises administering to the human (i) Compound 1, or the prodrug of Compound 1, or a pharmaceutically acceptable salt and (ii) nirmatrelvir, ritonavir, or a combination of nirmatrelvir and ritonavir. In some embodiments, the method comprises administering Compound 1, or a pharmaceutically acceptable salt thereof. In some embodiments, the method comprises administering Compound 1. In some embodiments, the method comprises administering deuterated Compound 1, or a pharmaceutically acceptable salt thereof. In some embodiments, the method comprises administering deuterated Compound 1. In some embodiments, the method comprises administering a prodrug of deuterated Compound 1, or a pharmaceutically acceptable salt thereof. In some embodiments, the method comprises administering a prodrug of deuterated Compound 1. In some embodiments, the method comprises administering a prodrug of Compound 1, or a pharmaceutically acceptable salt thereof. In some embodiments, the method comprises administering a prodrug of Compound 1.

In some embodiments, the prodrug of Compound 1 is:

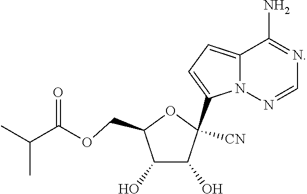 [SMILES: CC(C)C(=O)OC[C@H]1O[C@@](C#N)(c2ccc3c(N)ncnn23)[C@H](O)[C@@H]1O]

In some embodiments, upon administration to the human (i) Compound 1, or a pharmaceutically acceptable salt or prodrug thereof, and (ii) nirmatrelvir, ritonavir, or a combination of nirmatrelvir and ritonavir have a synergistic effect. In some embodiments, upon administration to the human (i) Compound 1, or a pharmaceutically acceptable salt or prodrug thereof, and (ii) nirmatrelvir, ritonavir, or a combination of nirmatrelvir and ritonavir have an additive effect. In some embodiments, upon administration to the human (i) Compound 1, or a pharmaceutically acceptable salt or prodrug thereof, and (ii) nirmatrelvir, ritonavir, or a combination of nirmatrelvir and ritonavir have non-antagonistic effect.

In some embodiments, the human is not a pregnant individual. In some embodiments, the human is not hospitalized. In some embodiments, the human is hospitalized.

In some embodiments, the human is administered nirmatrelvir and is not administered ritonavir. In some embodiments, the human is administered ritonavir and is not administered nirmatrelvir. In some embodiments, the human is administered both ritonavir and nirmatrelvir.

In some embodiments, nirmatrelvir is administered orally. In some embodiments, nirmatrelvir is administered twice daily. In some embodiments, nirmatrelvir is administered twice daily for a period of at least five days. In some embodiments, nirmatrelvir is administered twice daily for a period of five days. In some embodiments, nirmatrelvir is administered within 5 days of symptom onset. In some embodiments, nirmatrelvir is administered within 1 day, 2 days, 3 days, 4 days, or 5 days of symptom onset. In some embodiments, nirmatrelvir is administered in a dosage of 100 mg to 1,600 mg, 100 mg to 900 mg, 100 mg to 700 mg, 100 mg to 500 mg, 100 mg to 400 mg/dose, 200 mg to 1,600 mg, 200 mg to 900 mg, 200 mg to 800 mg, 200 mg to 700 mg, 200 mg to 500 mg, or 200 mg to 400 mg. In some embodiments, nirmatrelvir is administered in a dosage of about 300 mg. In some embodiments, the dosage comprises two tablets of about 150 mg. In some embodiments, the dosage of about 300 mg is administered twice daily. In some embodiments, the human is administered 200 mg to 3,200 mg, 200 mg to 1,800 mg, 200 mg to 1,400 mg, 200 mg to 1,000 mg, 200 mg to 800 mg/dose, 400 mg to 3,200 mg, 400 mg to 1,800 mg, 400 mg to 1,600 mg, 400 mg to 1,400 mg, 400 mg to 1,000 mg, or 400 mg to 800 mg of nirmatrelvir per day. In some embodiments, the human is administered about 600 mg of nirmatrelvir per day.

In some embodiments, ritonavir is administered orally. In some embodiments, ritonavir is administered twice daily. In some embodiments, ritonavir is administered twice daily for at least a period of five days. In some embodiments, ritonavir is administered twice daily for a period of five days. In some embodiments, ritonavir is administered within 5 days of symptom onset. In some embodiments, ritonavir is administered within 1 day, 2 days, 3 days, 4 days, or 5 days of symptom onset. In some embodiments, ritonavir is administered in a dosage of 25 mg to 800 mg, 25 mg to 600 mg, 25 mg to 400 mg, 25 mg to 300 mg, 25 mg to 150 mg, 50 mg to 800 mg, 50 mg to 700 mg, 50 mg to 600 mg, 50 mg to 400 mg, 50 mg to 300 mg, or 50 mg to 150 mg. In some embodiments, ritonavir is administered in a dosage of about 100 mg. In some embodiments, the dosage comprises one tablet of about 100 mg. In some embodiments, the dosage of about 100 mg is administered twice daily. In some embodiments, the human is administered 50 mg to 1,600 mg, 50 mg to 1,200 mg, 50 mg to 800 mg, 50 mg to 600 mg, 50 mg to 300 mg, 100 mg to 1,600 mg, 100 mg to 1,400 mg, 100 mg to 1,200 mg, 100 mg to 800 mg, 100 mg to 600 mg, or 100 mg to 300 mg of ritonavir per day. In some embodiments, the human is administered about 200 mg of ritonavir per day.

In some embodiments, nirmatrelvir and ritonavir are administered orally. In some embodiments, nirmatrelvir and ritonavir are administered twice daily. In some embodiments, nirmatrelvir and ritonavir are administered twice daily for at least period of five days. In some embodiments, nirmatrelvir and ritonavir are administered twice daily for a period of five days. In some embodiments, nirmatrelvir and ritonavir are administered within 5 days of symptom onset. In some embodiments, nirmatrelvir and ritonavir are administered within 1 day, 2 days, 3 days, 4 days, or 5 days of symptom onset. In some embodiments, nirmatrelvir is administered in a dosage of 100 mg to 1,600 mg, 100 mg to 900 mg, 100 mg to 700 mg, 100 mg to 500 mg, 100 mg to 400 mg/dose, 200 mg to 1,600 mg, 200 mg to 900 mg, 200 mg to 800 mg, 200 mg to 700 mg, 200 mg to 500 mg, or 200 mg to 400 mg and the ritonavir is administered in a dosage of 25 mg to 800 mg, 25 mg to 600 mg, 25 mg to 400 mg, 25 mg to 300 mg, 25 mg to 150 mg, 50 mg to 800 mg, 50 mg to 700 mg, 50 mg to 600 mg, 50 mg to 400 mg, 50 mg to 300 mg, or 50 mg to 150 mg. In some embodiments, nirmatrelvir is administered in a dosage of about 300 mg and ritonavir is administered in a dosage of about 100 mg. In some embodiments, the nirmatrelvir dosage comprises two tablets of about 150 mg and the ritonavir dosage comprises one tablet of about 100 mg. In some embodiments, the nirmatrelvir dosage of about 300 mg and the ritonavir dosage of about 100 mg are administered twice daily. In some embodiments, the human is administered 200 mg to 3,200 mg, 200 mg to 1,800 mg, 200 mg to 1,400 mg, 200 mg to 1,000 mg, 200 mg to 800 mg/dose, 400 mg to 3,200 mg, 400 mg to 1,800 mg, 400 mg to 1,600 mg, 400 mg to 1,400 mg, 400 mg to 1,000 mg, or 400 mg to 800 mg of nirmatrelvir per day and about 50 mg to 1,600 mg, 50 mg to 1,200 mg, 50 mg to 800 mg, 50 mg to 600 mg, 50 mg to 300 mg, 100 mg to 1,600 mg, 100 mg to 1,400 mg, 100 mg to 1,200 mg, 100 mg to 800 mg, 100 mg to 600 mg, or 100 mg to 300 mg of ritonavir per day. In some embodiments, the human is administered about 600 mg of nirmatrelvir per day and about 200 mg of ritonavir per day.

In some embodiments, the viral infection is a coronavirus infection. In some embodiments, the viral infection is a Severe Acute Respiratory Syndrome (SARS-CoV) infection, a Middle Eastern Respiratory Syndrome (MERS) infection, or a SARS-CoV-2 infection (COVID19). In some embodiments, the viral infection is a SARS-CoV-2 infection (COVID19).

The invention will be described in greater detail by way of specific examples. The following examples are offered for illustrative purposes and are not intended to limit the invention in any manner. Those of skill in the art will readily recognize a variety of non-critical parameters, which can be changed or modified to yield essentially the same results.

### EXAMPLES

The invention is further described in the following examples, which do not limit the scope of the invention described in the claims.

### Example 1. Nonclinical Study—Toxicology Study: An Enhanced Dose Range-Finding and Toxicokinetic Embryo-Fetal Development Study of Compound 16 by Oral Gavage Administration in Wistar Han Rats

The effects of compound 16 on pregnant Wistar Han rats and development of the embryo consequent to exposure of the female from implantation to closure of the hard palate were evaluated. Compound 16 was administered to 4 groups of presumed pregnant rats (8/group) at doses of 0 (vehicle), 125, 250, and 500 mg/kg/day via once daily gavage administration during organogenesis (gestation days [GD] 6 to 17) at a dose volume of 5 mL/kg. Additional pregnant rats were similarly dosed for toxicokinetic assessment of compound 16 and compound 1. Assessment of toxicity to the pregnant rats was based on mortality, clinical observations, body weights and food consumption. Rats were necropsied and cesarean sectioned on GD 21. An examination of the external features of the carcass; external body orifices; abdominal, thoracic, pelvic, and oral cavities; organs; and tissues was performed. Any macroscopic abnormalities were noted. Pregnancy status was determined. The uterus from each pregnant animal was excised, weighed, and examined for the number and placement of live and dead fetuses, the number of early or late resorptions, and any abnormalities. The right and left ovaries from each pregnant female were examined for the number of corpora lutea. Each fetus was sexed, weighed, and examined for external abnormalities. Each fetus had their heads removed, stored frozen on dry ice, and cross-sectioned using the Wilson's sectioning technique (Astroff et al., 2002). Internal organs of the thoracic and abdominal cavities were examined in the fresh state using modified Staples' technique (Stuckhardt and Poppe, 1984). Fetal findings were classified as variations or malformations.

The dose level of 500 mg/kg/day was not tolerated and the toxicity dose group was terminated early by Gestation Day (GD) 16. Mortality, severe clinical signs, and macroscopic observations at necropsy of enlarged and discolored kidneys, and enlarged and discolored adrenal glands were expected findings as they had already been observed in previous toxicology studies in female rats. Due to excessive maternal toxicity, fetal parameters were not evaluated for this group.

There were no remarkable clinical signs or effects on body weight, food consumption or macroscopic observations at 125 or 250 mg/kg/day. No compound 16-related effects were observed for embryo-fetal viability parameters, litter and fetal weights, or fetal pathology at 125 or 250 mg/kg/day. The no-observed-adverse-effect level (NOAEL) for maternal toxicity and embryofetal development was 250 mg/kg/day, associated with compound 1 AUC0-24h exposures of 183,000 ng*h/mL on GD 17.

### Example 2. Nonclinical Study—Toxicology Study: An Embryo-Fetal Development Study of Compound 16 by Oral Gavage in Wistar Han Rats

The potential of compound 16 to induce developmental toxicity after maternal exposure during the critical period of organogenesis (GD 6 through 17) were evaluated, maternal toxicity at the exposure levels tested was characterized, and a no observed-adverse-effect level (NOAEL) for maternal toxicity and developmental toxicity following oral administration in Wistar Han rats was determined.

Compound 16 was administered to 3 groups of presumed pregnant rats (25/group) at doses of 0 (vehicle), 125 and 250 mg/kg/day via once daily gavage administration during organogenesis (gestation days [GD] 6 to 17) at a dose volume of 5 mL/kg. Assessment of toxicity to the pregnant rats was based on mortality, clinical observations, body weights and food consumption. Rats were necropsied and cesarean sectioned on GD 21. An examination of the external features of the carcass; external body orifices; abdominal, thoracic, pelvic, and oral cavities; organs; and tissues was performed. Any macroscopic abnormalities were noted. Pregnancy status was determined. The uterus from each pregnant animal was excised, weighed, and examined for the number and placement of live and dead fetuses, the number of early or late resorptions, and any abnormalities. The right and left ovaries from each pregnant female were examined for the number of corpora lutea. Each fetus was sexed, weighed, and examined for external abnormalities Approximately one-half of the fetuses in each litter were examined for visceral anomalies by dissection in the fresh state. The thoracic and abdominal cavities were opened and dissected using a technique described by Stuckhardt and Poppe (1984). Fetal kidneys were examined and graded for renal papillae development (Woo and Hoar, 1972). The heads from these fetuses were removed and placed in Harrison's fixative for soft tissue examination using the Wilson sectioning technique (Wilson, 1965). The remaining fetuses (approximately one-half from each litter), were eviscerated, fixed in 100% ethyl alcohol, macerated in potassium hydroxide and stained with Alizarin Red S by a method similar to that described by Dawson (1926) for subsequent examination of skeletons.

No compound 16-related effects on intrauterine growth or survival were noted. No compound 16-related external, visceral or skeletal malformations or variations were noted. The no-observed-adverse-effect level (NOAEL) for maternal toxicity and embryofetal development was 250 mg/kg/day.

### Example 3. Nonclinical Study—Toxicology Study: An Oral Gavage Embryo-Fetal Development and Toxicokinetic Study with Compound 16 in Rabbits

The effects of compound 16 on pregnant New Zealand White (NZW) rabbits and the development of the embryo consequent to exposure of the female from implantation to closure of the hard palate (Gestation Days [GD] 7 through 19) was evaluated. Compound 16 was administered to 4 groups of pregnant rabbits (20/group) at doses of 0 (0.5% methylcellulose in deionized water), 125, 250, and 500 mg/kg/day once daily via oral gavage during organogenesis (GD 7 to 19). Assessment of toxicity was based on mortality, clinical observations, body weights, food consumption, and necropsy and cesarean section findings. In addition, blood samples were collected for toxicokinetic evaluation of compound 16 and compound 1 from similarly dosed animals (4/group). Due to excessive maternal toxicity observed at 500 mg/kg/day, the toxicity dose group was terminated early between GD 17 and 22, and fetal parameters were not evaluated for this group.

Reduced food consumption compared to control was noted for animals administered 250 or 500 mg/kg/day, with a decrease in mean maternal weight gain relative to control in animals administered 250 mg/kg/day, and mean body weight loss relative to GD 7 weight in animals administered 500 mg/kg/day. An increase in the rate of post-implantation loss was noted in animals administered 250 mg/kg/day. Increased rates of post implantation loss for animals administered 250 mg/kg/day correlated with an increase in the mean number of early resorptions and a reduction in the mean number of live fetuses. Fetuses from animals administered 250 mg/kg/day were noted with increased rates of ventricular septum defects, large or small heart ventricles, dilated or malpositioned aortas, retroesophageal aortic arches, retroesophageal subclavian arteries, abnormal lobulation of the liver. No compound 16-related skeletal malformations were noted. Based on these findings, the NOAEL of compound 16 for embryo-fetal development in rabbits was 125 mg/kg/day associated with compound 1 AUC0-24h exposures of 64,100 ng*h/mL on GD 17.

### Example 4. In Vitro Combination Analysis of SARS-CoV-2 Antivirals in Clinical Use with Compound 1/Compound 16

Materials and Methods

Compounds

All compounds were synthesized by known methods or purchased. Validation of chemical identities were determined by NMR and LCMS, and purity>95% was assessed by HPLC. Compounds were solubilized in 100% dimethyl sulfoxide (DMSO) at a concentration of 10 mM.

Cells

A549-hACE2 cells that stably express human angiotensin converting enzyme 2 (hACE2) were established and provided by the University of Texas Medical Branch (Mossel, E C 2005). A549-hACE2 cells were maintained at 37° C. and 5% CO₂ in Dulbecco's Minimum Essential Medium (DMEM) with GlutaMAX (Gibco cat #10569-010) supplemented with 10% heat-inactivated fetal bovine serum (FBS) (Hyclone Cat #SH30396.03), 100 units/mL penicillin, 100 μg/mL streptomycin (Gibco Cat #15140-122), and the selection agent—10 μg/mL Blasticidin. A549-hACE2 cells used in all experimental set-ups were between passage 5 and 30.

Virus Propagation

Recombinant firefly luciferase WA SARS-CoV-2 virus (SARS-CoV-2 Fluc) was amplified from a stock obtained from University of Texas Medical Branch (UTMB; Galveston, TX) generated as described previously (Xie, X., et al. (2021). “Engineering SARS-CoV-2 using a reverse genetic system.” Nat Protoc 16(3): 1761-1784). SARS-CoV-2 Fluc was propagated as high titer stocks in Vero-TMPRSS2 cells as follows: 1×10⁷ Vero-TMPRSS2 cells were seeded into a T225 flask in Vero-TMPRSS2 maintenance media and incubated overnight at 37° C.+5% CO₂. The following day the media was aspirated and replaced with 25 mL of DMEM supplemented with 2% FBS (infection medium) and infected with 10 μL of a P0 SARS-CoV-2 Fluc stock. The flasks were returned to 37° C.+5% CO₂ until only 10-20% of viable cells remained (typically 36-72 hours post infection (hpi)). The supernatant was harvested into a 50 mL falcon tube and centrifuged at 2000×g for 5 minutes to pellet cellular debris. The clarified supernatant was then aliquoted as a working P1 stock into 250 μL aliquots and frozen at −80° C. The titer of the working P1 stock was determined by plaque formation assay (PFA).

SARS-CoV-2 Fluc Antiviral Assay

A549-hACE2 cells were plated at 12,000 cells per well, in 96-well plates, in 100 μL volume of maintenance media and incubated overnight at 37° C. with 5% CO₂. The following day spent media was aspirated and 100 μL of infection media (DMEM supplemented with 2% FBS) was added to each well. Compounds were added directly to cultures using a HP D300e digital liquid dispenser, with DMSO volumes normalized to that of the highest compound concentration (final DMSO concentration<0.1%). Plates (in quadruplicate for each combination) were laid out in an array with increasing concentrations across rows and columns of the two drugs being assessed and compound dilution placement was then randomized on each plate to reduce variability from edge effect. Compound 16, Compound 1, and molnupiravir were dispensed into plates at starting concentrations of 4000, 5000, and 10,000 nM, respectively and titrated by with a pre-determined optimal serial dilution for each compound. Nirmatrelvir was dispensed into plates at a starting concentration or 600 nM and serially diluted 1:2. For the 3-drug combinations, compounds were dispensed as described above, but ritonavir was dispensed at either 0, 300, 600, 1200, or 2400 nM for the entirety of individual plates, in addition to 2 other compounds being assessed (Compound 16 and nirmatrelvir or Compound 1 and nirmatrelvir). 50 μL of recombinant WA SARS-CoV-2 Fluc was added at a MOI of 1 and for uninfected control wells, 50 μL of infection media was added to one column and cultures were incubated at 37° C. with 5% CO₂. At 24 hpi, cultures were removed from the incubator, and 50 μL of Promega One-Glo Luciferase Assay System (CAT #E6120) was added to all wells. Plates were put on the orbital shaker for 10 minutes and read for luminescence on the Perkin Elmer Envision plate reader.

Synergy Finder 3.0 Analysis

After de-randomizing the raw luminescence data from the Envision plate reader in Microsoft Excel, dose-dependent percent inhibition of WA SARS-CoV-2 Fluc was calculated (% inhibition=100×(1−(X−min)/(max−min)), and the data matrices were uploaded into Synergy Finder (version 3.0) (https://synergyfinder.fimm.fi) analytic software {Ianevski, A 2022 #67038}. The drug combination effects were quantified using Bliss independence (background corrected) mathematical models. Synergy reports were generated for each drug combination, and scores are reported as median values of all four biological replicates using the conventional Synergy scoring methodology. The methodology defines a synergy score between −10 and 10 as being additive, below −10 is considered antagonistic, and above 10 is considered synergistic {Ianevski, A 2022 #67038}.

Results

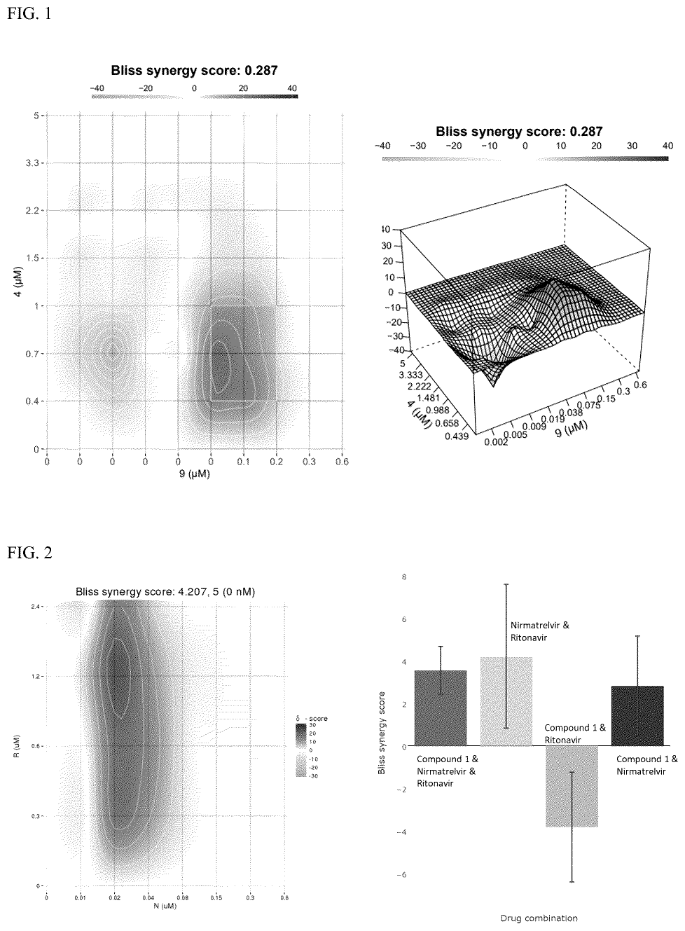

Bliss independence consensus scores were used to evaluate the drug combinations below by interpreting any score between −10 and 10 as additive, greater than 10 are synergistic, and below 10 are antagonistic. The Bliss consensus score summary for 2-drug combinations is reported in Table 4.1 and Table 4.2. For 2-drug combinations of Compound 1 and nirmatrelvir, it was observed that an additive effect with an average Bliss score of 0.96±0.67 for four replicates (representative Bliss independence plots are presented in FIG. 1). For Compound 16 and nirmatrelvir, it was observed that additive effects with and average Bliss score of 2.77±0.07 across replicates. Additive effects are observed for the combinations of Compound 1 with molnupiravir and Compound 16 with molnupiravir, demonstrated by average Bliss scores of −0.22±0.20 and −1.36±1.44, respectively. Alongside these 2-drug combinations, Compound 1, Compound 16, nirmatrelvir, and molnupiravir were tested as self-self combinations to serve as assay validation controls. Compounds cannot be synergistic or antagonistic with themselves, and the assay verified additive drug self-self-combination effects for all the drugs evaluated. Additive effects were observed for Compound 1, Compound 16, nirmatrelvir, and molnupiravir self-self combinations with Bliss scores of 8.00±1.09, 6.15±2.16, 0.13±1.53, and −2.47 2.10, respectively as shown in Table 4.2.

The 3-drug combination Bliss consensus scores are summarized in Table 4.3 and Table 4.4. For the 3-drug combinations of Compound 1, nirmatrelvir, and ritonavir, additive effects are observed with an average Bliss score of 2.80±3.95. FIG. 2 illustrates a representative Bliss independence plot for this combination. Alongside the triple drug combination, 2-drug combinations of nirmatrelvir with ritonavir, Compound 1 with ritonavir, and Compound 1 with nirmatrelvir demonstrate additive effects with average Bliss scores of 0.63±5.65, −2.47±3.00, and 1.67 0.53, respectively. Likewise, 3-drug combinations of Compound 16, nirmatrelvir, and ritonavir, demonstrate additive effects with an observed average Bliss score of 5.33±4.76 across replicates. Alongside this 3-drug combination, 2-drug combinations of nirmatrelvir with ritonavir, Compound 16 with ritonavir, and Compound 16 with nirmatrelvir, demonstrate additive effects with observed average Bliss scores of 6.31±5.38, 0.75±6.96, and 2.01±0.79, respectively.

TABLE 4.1
Two-Drug Combination Bliss Independence Consensus Scores
for the A549-hACE-2 SARS-CoV-2 Fluc Antiviral Analyses

| | 2 Compound Combinations | | | |
|---|---|---|---|---|
| | Compound 1 + | Compound 16 + | Compound 1 + | Compound 16 + |
| | Nirmatrelvir | Nirmatrelvir | Molnupiravir | Molnupiravir |
| Replicate 1 | 1.622 | 2.782 | −0.414 | −1.328 |
| Replicate 2 | 0.287 | 2.703 | −0.013 | 0.059 |
| Replicate 3 | 0.977 | 2.836 | −0.228 | −2.814 |
| Replicate 4 | N/A | N/A | N/A. | N/A. |
| Average of | 0.96 | 2.77 | −0.22 | −1.36 |
| Replicates | | | | |
| Standard | 0.67 | 0.07 | 0.20 | 1.44 |
| Deviation of | | | | |
| Replicates | | | | |

TABLE 4.2
Two-Drug Self-Self Combination Bliss Independence Consensus
Scores for the A549-hACE-2 SARS-CoV-2 Fluc Antiviral Analyses

| | 2 Compound Combinations | | | |
|---|---|---|---|---|
| | Compound 1 + | Nirmatrelvir + | Compound 16 + | Molnupiravir + |
| | Compound 1 | Nirmatrelvir | Compound 16 | Molnupiravir |
| Replicate 1 | 9.602 | −1.963 | 3.653 | −1.127 |
| Replicate 2 | 7.305 | 0.251 | 7.235 | −4.887 |
| Replicate 3 | 7.302 | 1.692 | 7.548 | −1.404 |
| Replicate 4 | 7.776 | 0.555 | N/A. | N/A. |
| Average of | 8.00 | 0.13 | 6.15 | −2.47 |
| Replicates | | | | |
| Standard | 1.09 | 1.53 | 2.16 | 2.10 |
| Deviation of | | | | |
| Replicates | | | | |

TABLE 4.3
Three-Drug Combination Compound 1 Bliss Independence Consensus
Scores for the A549-hACE-2 SARS-CoV-2 Fluc Antiviral Analyses

| | 3 Compound Combinations | | | |
|---|---|---|---|---|
| | Compound 1 + | | | |
| | Nirmatrelvir + | Nirmatrelvir + | Compound 1 + | Compound 1 + |
| | Ritonavir | Ritonavir | Ritonavir | Nirmatrelvir |
| Replicate 1 | 4.122 | 1.994 | −1.189 | 2.135 |
| Replicate 2 | 5.923 | 5.479 | −0.322 | 1.77 |
| Replicate 3 | −1.637 | −5.578 | −5.898 | 1.098 |
| Average of | 2.80 | 0.63 | −2.47 | 1.67 |
| Replicates | | | | |
| Standard | 3.95 | 5.65 | 3.00 | 0.53 |
| Deviation of | | | | |
| Replicates | | | | |

TABLE 4.4
Three-Drug Combination Compound 16 Bliss Independence Consensus
Scores for the A549-hACE-2 SARS-CoV-2 Fluc Antiviral Analyses

| | 3 Compound Combinations | | | |
|---|---|---|---|---|
| | Compound 16 + | | | |
| | Nirmatrelvir + | Nirmatrelvir + | Compound 16 + | Compound 16 + |
| | Ritonavir | Ritonavir | Ritonavir | Nirmatrelvir |
| Replicate 1 | 1.714 | 2.309 | −2.675 | 1.244 |
| Replicate 2 | 3.546 | 4.207 | −3.827 | 2.813 |
| Replicate 3 | 10.726 | 12.426 | 8.766 | 1.958 |
| Average of | 5.33 | 6.31 | 0.75 | 2.01 |
| Replicates | | | | |
| Standard | 4.76 | 5.38 | 6.96 | 0.79 |
| Deviation of | | | | |
| Replicates | | | | |

Drug combinations of Compound 1 or Compound 16 with either molnupiravir, nirmatrelvir, and/or ritonavir all demonstrate additive effects against SARS-CoV-2 in A549-hACE-2 cells.

### Example 5. Two-Drug Combination Loewe Model Scores for the A549-hACE-2 SARS-CoV-2 Fluc Antiviral Analyses

Materials and Methods

Compounds

All compounds were synthesized by known methods or purchased. Validation of chemical identities were determined by NMR and LCMS, and purity>95% was assessed by HPLC. Compounds were solubilized in 100% dimethyl sulfoxide (DMSO) at a concentration of 10 mM.

Cells

A549-hACE2 cells that stably express human angiotensin converting enzyme 2 (hACE2) were established and provided by the University of Texas Medical Branch (Mossel, E C 2005). A549-hACE2 cells were maintained at 37° C. and 5% CO₂ in Dulbecco's Minimum Essential Medium (DMEM) with GlutaMAX (Gibco cat #10569-010) supplemented with 10% heat-inactivated fetal bovine serum (FBS) (Hyclone Cat #SH30396.03), 100 units/mL penicillin, 100 μg/mL streptomycin (Gibco Cat #15140-122), and the selection agent—10 μg/mL Blasticidin. A549-hACE2 cells used in all experimental set-ups were between passage 5 and 30.

Virus Propagation

Recombinant firefly luciferase WA SARS-CoV-2 virus (SARS-CoV-2 Fluc) was amplified from a stock obtained from University of Texas Medical Branch (UTMB; Galveston, TX) generated as described previously (Xie, X., et al. (2021). “Engineering SARS-CoV-2 using a reverse genetic system.” Nat Protoc 16(3): 1761-1784). SARS-CoV-2 Fluc was propagated as high titer stocks in Vero-TMPRSS2 cells as follows: 1×10⁷ Vero-TMPRSS2 cells were seeded into a T225 flask in Vero-TMPRSS2 maintenance media and incubated overnight at 37° C.+5% CO₂. The following day the media was aspirated and replaced with 25 mL of DMEM supplemented with 2% FBS (infection medium) and infected with 10 μL of a P0 SARS-CoV-2 Fluc stock. The flasks were returned to 37° C.+5% CO₂ until only 10-20% of viable cells remained (typically 36-72 hours post infection (hpi)). The supernatant was harvested into a 50 mL Falcon tube and centrifuged at 2000×g for 5 minutes to pellet cellular debris. The clarified supernatant was then aliquoted as a working P1 stock into 250 μL aliquots and frozen at −80° C. The titer of the working P1 stock was determined by plaque formation assay (PFA).

SARS-CoV-2 Fluc Antiviral Assay

A549-hACE2 cells were plated at 12,000 cells per well, in 96-well plates, in 100 μL volume of maintenance media and incubated overnight at 37° C. with 5% CO₂. The following day spent media was aspirated and 100 μL of infection media (DMEM supplemented with 2% FBS) was added to each well. Compounds were added directly to cultures using a HP D300e digital liquid dispenser, with DMSO volumes normalized to that of the highest compound concentration (final DMSO concentration<0.1%). Plates (in quadruplicate for each combination) were laid out in an array with increasing concentrations across rows and columns of the two drugs being assessed and compound dilution placement was then randomized on each plate to reduce variability from edge effect. 50 μL of recombinant WA SARS-CoV-2 Fluc was added at a MOI of 1 and for uninfected control wells, 50 μL of infection media was added to one column and cultures were incubated at 37° C. with 5% CO₂. At 24 hpi, cultures were removed from the incubator, and 50 μL of Promega One-Glo Luciferase Assay System (CAT #E6120) was added to all wells. Plates were put on the orbital shaker for 10 minutes and read for luminescence on the Perkin Elmer Envision plate reader.

Synergy Finder Plus Analysis

After de-randomizing the raw luminescence data from the Envision plate reader in Microsoft Excel, dose-dependent percent inhibition of WA SARS-CoV-2 Fluc was calculated (% inhibition=100×(1−(X−min)/(max−min)), and the data matrices were uploaded into SynergyFinder Plus) (DOI: 10.1016/j.gpb.2022.01.004) analytic software {Ianevski, A 2022 #67038}. The drug combination effects were quantified by the Loewe method. Synergy reports were generated for each drug combination. The methodology defines a synergy score between −10 and 10 as being additive, below −10 is considered antagonistic, and above 10 is considered synergistic {Ianevski, A 2022 #67038}. Results are shown in Table 5.1.

TABLE 5.1
Two-Drug Combination Loewe Model Scores for the
A549-hACE-2 SARS-CoV-2 Fluc Antiviral Analyses

| | 2 Compound Combinations | | |
|---|---|---|---|
| | Compound 1 + | Compound 1 + | Nirmatrelvir + |
| | Nirmatrelvir | Compound 1 | Nirmatrelvir |
| Replicate 1 | 0.35 | 1.34 | 0.80 |

### Example 6. Two-Drug Combination Loewe Model Scores for the A549-hACE-2 SARS-CoV-2 Fluc Antiviral Analyses in Non-Infectious BHK-SARS-2R_GFP_NeoR_NL (SCL414) Replicon Cell

Materials and Methods

Cell Culture

Non-infectious BHK-SARS-2R_GFP_NeoR_NL (SCL414) replicon cell lines were licensed from Emory University (Lan et al 2021). These are stable cell lines expressing SARS-CoV-2 replicon DNA and sNLuc reporter gene. Cells were maintained in Dulbecco's Modified Eagle's Medium (ThermoFisher, Catalog #10566016) supplemented with 10% fetal bovine serum ((ThermoFisher, Catalog #SH3040602HI), 100 U/mL penicillin/100 μg/mL streptomycin ((ThermoFisher, Catalog #15140122) and 1 mg/mL Geneticine™ ((ThermoFisher, Catalog #10131027).

Drug-Combination Assay

BHK-SARS-2R_GFP_NeoR_NL replicon cell line 50 μL of the assay media (DMEM supplemented with 10% FBS) was added to white clear bottom 96-well plates (ThermoFisher, Catalog #07-200-566). Serial dilutions of Compound 1 and nirmatrelvir were added in a matrix format to the wells with assay media using the HP D300e digital dispenser with normalization to the highest concentration of DMSO (ATCC 4-X™) in all wells (>0.1% final volume). Starting concentration for Compound 1 was 12 μM and for nirmatrelvir was 1.2 μM. For negative control, puromycin dissolved in DMSO was added at a final concentration of 10 μM. Untreated wells were used as a positive control. For this assay the drug combination matrix of Compound 1 with nirmatrelvir along with nirmatrelvir and Compound 1 self-matrix (internal control) was tested. Cells were then seeded in the same plate at a density of 8.0×10⁴ cells/mL for a total volume of 100 μL/well and incubated for three days at 37° C. with 5% CO₂. 50 μL/well of room temperature Nano-Glo® luciferase reagent (Promega, Catalog #N1110) was added to the plate. The plates were incubated for 1-2 mins on a shaker at room temperature and luminescence was measured using an Envision plate reader (PerkinElmer, Catalog #2105-0010). Each combination was performed in replicates of three. Average of the three replicates was used for analysis. Drug-response data were normalized to positive and negative control to determine percent inhibition.

Synergy Finder Plus Analysis

After de-randomizing the raw luminescence data from the Envision plate reader in Microsoft Excel, dose-dependent percent inhibition of WA SARS-CoV-2 Fluc was calculated (% inhibition=100×(1−(X−min)/(max−min)), and the data matrices were uploaded into SynergyFinder Plus) (DOI: 10.1016/j.gpb.2022.01.004) analytic software {Ianevski, A 2022 #67038}. The drug combination effects were quantified by the Loewe method. Synergy reports were generated for each drug combination. The methodology defines a synergy score between −10 and 10 as being additive, below −10 is considered antagonistic, and above 10 is considered synergistic {Ianevski, A 2022 #67038}. Results are shown in Table 6.1.

TABLE 6.1
Two-Drug Combination Loewe Model Scores for the
A549-hACE-2 SARS-CoV-2 Fluc Antiviral Analyses

| | 2 Compound Combinations | | |
|---|---|---|---|
| | Compound 1 + | Compound 1 + | Nirmatrelvir + |
| | Nirmatrelvir | Compound 1 | Nirmatrelvir |
| Replicate 1 | 1.19 | −1.38 | −1.05 |

All references, including publications, patents, and patent documents are incorporated by reference herein, as though individually incorporated by reference. The present disclosure provides reference to various embodiments and techniques. However, it should be understood that many variations and modifications may be made while remaining within the spirit and scope of the present disclosure. The description is made with the understanding that it is to be considered an exemplification of the claimed subject matter and is not intended to limit the appended claims to the specific embodiments illustrated.

1. A method of treating a viral infection in a patient in need thereof, wherein the method comprises administering to the patient Compound 16: 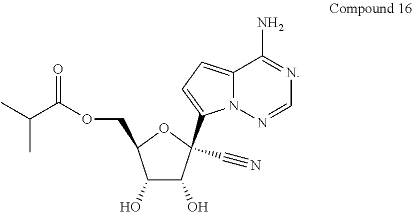 [SMILES: CC(C)C(=O)OC[C@H]1O[C@@](C#N)(c2ccc3c(N)ncnn23)[C@H](O)[C@@H]1O]
or a pharmaceutically acceptable salt thereof,
wherein the patient is not a pregnant individual.

2.-7. (canceled)

8. The method of claim 1, wherein Compound 16, or a pharmaceutically acceptable salt thereof, is administered orally.

9. The method of claim 1, wherein Compound 16, or a pharmaceutically acceptable salt thereof, is administered once daily.

10. The method of claim 1, wherein Compound 16, or a pharmaceutically acceptable salt thereof, is administered twice daily.

11. (canceled)

12. (canceled)

13. The method of claim 9, wherein Compound 16, or a pharmaceutically acceptable salt thereof, is administered for 3 to 14 consecutive days.

14. The method of claim 1, wherein Compound 16, or a pharmaceutically acceptable salt thereof, is administered once daily for three consecutive days.

15. The method of claim 1, wherein Compound 16, or a pharmaceutically acceptable salt thereof, is administered twice daily for three consecutive days.

16. The method of claim 1, wherein Compound 16, or a pharmaceutically acceptable salt thereof, is administered once daily for five consecutive days.

17. The method of claim 1, wherein Compound 16, or a pharmaceutically acceptable salt thereof, is administered twice daily for five consecutive days.

18. The method of claim 1, wherein the patient is not pregnant on the first day of the administration of Compound 16, or a pharmaceutically acceptable salt thereof, and the patient avoids becoming pregnant from the first day to at least 2 days after the administration.

19.-22. (canceled)

23. The method of claim 1, wherein the patient is a lactating individual, and the patient avoids breastfeeding from the first day of the administration of Compound 16, or a pharmaceutically acceptable salt thereof, to at least 2 days after the administration.

24.-26. (canceled)

27. The method of claim 1, wherein the patient is a lactating individual, and the patient avoids breastfeeding from the first day of the administration of Compound 16, or a pharmaceutically acceptable salt thereof, to at least one month after the administration.

28. (canceled)

29. The method of claim 1, further comprising determining that the patient is not pregnant before administering Compound 16, or a pharmaceutically acceptable salt thereof.

30. (canceled)

31. (canceled)

32. The method of claim 1, wherein the patient is potentially child-bearing, and wherein the patient has had at least one negative pregnancy test at least one day prior to the administration of Compound 16, or a pharmaceutically acceptable salt thereof.

33.-37. (canceled)

38. The method of claim 32, wherein each pregnancy test is independently a urine pregnancy test or a serum pregnancy test.

39. (canceled)

40. The method of claim 1, wherein the patient is potentially child-bearing, and wherein the patient uses at least one form of contraception from the first day of the administration of Compound 16, or a pharmaceutically acceptable salt thereof, to at least 2 days after the administration.

41.-43. (canceled)

44. The method of claim 40, wherein the patient uses at least one form of contraception selected from tubal ligation, partner's vasectomy, intrauterine devices, birth control pills, injectable hormonal birth control products, implantable hormonal birth control products, insertable hormonal birth control products, diaphragms, latex condoms, cervical caps, and a spermicide.

45.-210. (canceled)

211. A method of treating a viral infection in a human in need thereof, the method comprising administering to the human
(i) Compound 1: 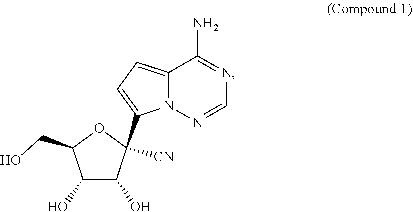 [SMILES: N#C[C@@]1(c2ccc3c(N)ncnn23)O[C@H](CO)[C@@H](O)[C@H]1O]
a deuterated Compound 1, a prodrug of Compound 1, a prodrug of deuterated Compound 1, or a pharmaceutically acceptable salt thereof; and
(ii) nirmatrelvir, ritonavir, or a combination of nirmatrelvir and ritonavir;
wherein when the prodrug of Compound 1, the prodrug of deuterated Compound 1, or the pharmaceutically acceptable salt thereof is administered to the human, the prodrug of Compound 1, the prodrug of deuterated Compound 1, or the pharmaceutically acceptable salt thereof is converted substantially to Compound 1 or deuterated Compound 1.

212. The method of claim 211, comprising administering to the human (i) Compound 1, or the prodrug of Compound 1, or a pharmaceutically acceptable salt and (ii) nirmatrelvir, ritonavir, or a combination of nirmatrelvir and ritonavir.

213. The method of claim 211, comprising administering Compound 1, or a pharmaceutically acceptable salt thereof.

214. The method of claim 211, comprising administering Compound 1.

215.-218. (canceled)

219. The method of claim 211, comprising administering a prodrug of Compound 1, or a pharmaceutically acceptable salt thereof.

220. The method of claim 211, comprising administering a prodrug of Compound 1.

221. The method of claim 211, comprising administering a prodrug of Compound 1 wherein the prodrug of Compound 1 is:

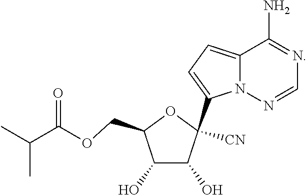 [SMILES: CC(C)C(=O)OC[C@H]1O[C@@](C#N)(c2ccc3c(N)ncnn23)[C@H](O)[C@@H]1O]

222. The method of claim 211, wherein the human is not a pregnant individual.

223.-229. (canceled)

230. The method of claim 211, wherein the human is administered both ritonavir and nirmatrelvir.

231.-254. (canceled)

255. The method of claim 211, wherein nirmatrelvir and ritonavir are administered orally.

256. The method of claim 211, wherein nirmatrelvir and ritonavir are administered twice daily.

257. (canceled)

258. (canceled)

259. The method of claim 211, wherein nirmatrelvir and ritonavir are administered within 5 days of symptom onset.

260.-266. (canceled)

267. The method of claim 211, wherein the viral infection is a coronavirus infection.

268. (canceled)

269. The method of claim 211, wherein the viral infection is a SARS-CoV-2 infection (COVID19).
